(19) United States
(12) Patent Application Publication
(10) Pub. No.: US 2016/0214974 A1
(43) Pub. Date: Jul. 28, 2016

(54) INSECTICIDAL COMPOUNDS

(71) Applicant: SYNGENTA PARTICIPATIONS AG, Basel (CH)

(72) Inventors: Jurgen Harry Schaetzer, Stein (CH); Fides Benfatti, Stein (CH); Jagadish Pabba, Goa (IN); Sebastian Rendler, Stein (CH); Andrew Crossthwaite, Berkshire (GB)

(73) Assignee: SYNGENTA PARTICIPATIONS AG, Basel (CH)

(21) Appl. No.: 14/916,017

(22) PCT Filed: Aug. 20, 2014
(86) PCT No.: PCT/EP2014/067701

§ 371 Date: Mar. 2, 2016

(30) Foreign Application Priority Data
Sep. 6, 2013 (IN) 2644/DEL/2013

(51) Int. Cl. C07D 451/02; A01N 43/90

(52) U.S. Cl. CPC C07D 451/02; A01N 43/90

(57) ABSTRACT

A compound of formula I, (I), wherein A, Q, R¹, and R², are as defined in claim 1. Furthermore, the present invention relates to intermediates used to prepare compounds of formula (I), to methods of using them to combat and control insect, acarine, nematode and mollusc pests and to insecticidal, acaricidal, nematicidal and molluscicidal compositions comprising them.

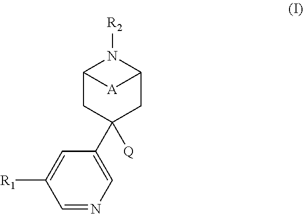

The present invention relates to new bicyclic amine derivatives, to processes for preparing them, to pesticidal, in particular insecticidal, acaricidal, molluscicidal and nematicidal compositions comprising them and to methods of using them to combat and control pests such as insect, acarine, mollusc and nematode pests.

Bicyclic amine derivatives with insecticidal properties are disclosed, for example, in WO9637494.

It has now surprisingly been found that certain novel bicyclic amine derivatives have favourable insecticidal properties.

The present invention therefore provides compounds of the formula I

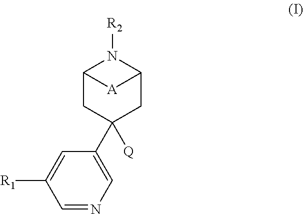

wherein
Q is —C(═S)NR³R⁴ or —C(═NR⁵)SR⁶; where R³ and R⁴ can independently of each other be selected from hydrogen, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), and where R⁵ and R⁶ are independently selected from hydrogen, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl);
A is —CH₂—CH₂— or —CH═CH—;
R¹ is halogen, cyano, C₁-C₃ alkoxy, C₃-C₅ cycloalkyl, —C≡CR⁷; where R⁷ is hydrogen, C₁-C₄alkyl, C₃-C₅cycloalkyl (optionally substituted by one to two substituents independently selected from halogen, methyl and C₁-C₂haloalkyl), tri(C₁-C₂)alkylsilyl; and
R² is hydrogen, formyl, cyano, hydroxy, NH₂, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₄alkoxyimino(C₁-C₄)alkyl, C₁-C₄haloalkoxy(C₁-C₄)alkyl, C₁-C₆alkoxycarbonyl(C₁-C₆)alkyl, C₁-C₄alkoxy(C₁-C₄)alkoxycarbonyl(C₁-C₆)alkyl, hydroxycarbonyl(C₁-C₆)alkyl, aryloxycarbonyl(C₁-C₆)alkyl (wherein the aryl group can be optionally substituted by one or two substituents independently selected from halogen, cyano, nitro, C₁-C₄alkyl, C₁-C₄ haloalkyl, and C₁-C₄alkoxy), C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, di(C₁-C₄haloalkyl)aminocarbonyl-C₁-C₆alkyl, C₁-C₂alkoxy(C₂-C₄)alkylaminocarbonyl(C₁-C₄)alkyl, C₂-C₆alkenyloxycarbonyl(C₁-C₆)alkyl, C₃-C₆alkynyloxycarbonyl(C₁-C₆)alkyl, (R⁸O)₂(O═)P(C₁-C₆)alkyl where R⁸ is hydrogen, C₁-C₄alkyl or benzyl, C₃-C₇cycloalkyl (optionally substituted by one to three substituents independently selected from C₁-C₄alkyl, C₁-C₄haloalkyl, and C₁-C₄alkoxy and, additionally, one of the ring member units can optionally represent C═O or C═NR⁹ where R⁹ is hydrogen, C₁-C₄alkyl, C₁-C₄haloalkyl, C₁-C₄cyanoalkyl, C₁-C₄alkoxy, or C₃-C₆cycloalkyl), C₃-C₇halocycloalkyl, C₃-C₇cycloalkenyl (optionally substituted by one or two substituents independently selected from C₁-C₄alkyl, and C₁-C₄haloalkyl, and, additionally, one of the ring member units can optionally represent C═O), C₃-C₇halocycloalkenyl, C₁-C₆alkyl-S(═O)n¹(C₁-C₆)alkyl where n¹ is 0, 1 or 2, C₃-C₆alkenyl, C₃-C₆haloalkenyl, aryl(C₃-C₆)alkenyl, C₃-C₆alkynyl, C₃-C₆haloalkynyl, aryl(C₃-C₆)alkynyl, C₃-C₆hydroxyalkynyl, C₁-C₆alkoxycarbonyl (optionally substituted by one to three substituents independently selected from halogen, hydroxy, cyano, C₁-C₄alkoxy, C₁-C₄haloalkyl, and aryl), aryloxycarbonyl (optionally substituted by one to three substituents independently selected from halogen, cyano, nitro, C₁-C₄alkyl, C₁-C₄haloalkyl, C₁-C₄alkoxy), C₃-C₆alkenyloxycarbonyl, C₃-C₆alkynyloxycarbonyl, C₁-C₆alkylcarbonyl, C₁-C₆haloalkylcarbonyl, aminocarbonyl, C₁-C₆alkylaminocarbonyl, di(C₁-C₆)alkylaminocarbonyl, aminothiocarbonyl, C₁-C₆alkylaminothiocarbonyl, di(C₁-C₆)alkylaminothiocarbonyl, C₁-C₆alkoxy, C₃-C₆alkenyloxy, C₃-C₈alkynyloxy, aryloxy (optionally substituted by one to three substituents independently selected from halogen, cyano, nitro, C₁-C₄alkyl, C₁-C₄haloalkyl, and C₁-C₄alkoxy), C₁-C₆alkylamino, di(C₁-C₆)alkylamino, C₃-C₆cycloalkylamino, C₁-C₄alkylthio, C₁-C₄alkylsulfinyl, C₁-C₄alkylsulfonyl, C₁-C₄haloalkylsulfonyl, aryl-S(═O)n² (optionally substituted by one or two substituents independently selected from halogen, nitro, and C₁-C₄alkyl) where n² is 0, 1 or 2, aryl (optionally substituted by one to three substituents independently selected from halogen, cyano, nitro, C₁-C₄alkyl, C₁-C₄haloalkyl, C₁-C₄alkoxy, and C₁-C₄haloalkoxy), heteroaryl (optionally substituted by one to three substituents independently selected from halogen, cyano, nitro, C₁-C₄alkyl, C₁-C₄haloalkyl, C₁-C₄alkoxy, and C₁-C₄haloalkoxy), heterocyclyl (optionally substituted by one to three substituents independently selected from halogen, cyano, nitro, C₁-C₄alkyl, C₁-C₄haloalkyl, C₁-C₄alkoxy, and C₁-C₄haloalkoxy, and, additionally, a ring member unit can optionally represent C═O or C═NR¹⁰ where R¹⁰ is hydrogen, C₁-C₄ alkyl, C₁-C₄ haloalkyl, C₁-C₄ cyanoalkyl, C₁-C₄ alkoxy, or C₃-C₆ cycloalkyl), (C₁-C₆alkylthio)carbonyl, (C₁-C₆alkylthio)thiocarbonyl, C₁-C₆alkyl-S(═O)n³(═NR¹¹)—C₁-C₄alkyl wherein R¹¹ is hydrogen, cyano, nitro, C₁-C₄alkyl and n³ is 0 or 1; or an agrochemically acceptable salt, N-oxide or isomer thereof.

There is a continuing need to find new methods of controlling insect populations, in particular insect populations which have developed resistance to one or more insecticides, as well as more selective methods of controlling insects whereby undesired insects are affected but beneficial arthropods are not affected, and additionally biologically active compounds suitable for use in the aforementioned methods, as well as new biologically active compounds displaying superior properties for use as agrochemical active ingredients (for example, greater biological activity, a different spectrum of activity, an increased safety profile, improved physico-chemical properties, or increased biodegradability).

The damage of plants, and in particular commercial crops, has resulted in large amounts of resources and efforts being spent attempting to control the activities of Hemiptera.

Plants exhibiting aphid damage can possess a variety of symptoms, such as decreased growth rates, mottled leaves, yellowing, stunted growth, curled leaves, browning, wilting, low yields and death. The removal of sap creates a lack of vigour in the plant, and aphid saliva can also be toxic to plants. Many Hemipteran species, transmit disease-causing organisms like plant viruses to their hosts. The green peach aphid (Myzus persicae) is a vector for more than 110 plant viruses. Cotton aphids (Aphis gossypii) are also vectors of several economically important viruses. Whiteflies feed by tapping into the phloem of plants, introducing toxic saliva and decreasing the plants' overall turgor pressure. Since whiteflies congregate in large numbers, susceptible plants can be quickly overwhelmed. Further harm is done by mold growth encouraged by the honeydew that both aphids and whiteflies secrete.

The neonicotinoids represent the fastest-growing class of insecticides introduced to the market since the commercialization of pyrethroids (Nauen & Denholm, 2005: Archives of Insect Biochemistry and Physiology 58:200-215) and are extremely valuable insect control agents not least because they had exhibited little or no cross-resistance to older insecticide classes, which suffer markedly from resistance problems. However, reports of insect resistance to the neonicotinoid class of insecticides are on the increase.

The increase in resistance of such insects to neonicotinoid insecticides thus poses a significant threat to the cultivation of a number of commercially important crops, fruits and vegetables, and there is thus a need to find alternative insecticides capable of controlling neonicotinoid resistant insects (i.e. to find insecticides that do not exhibit any cross-resistance with the neonicotinoid class).

Resistance may be defined as “a heritable change in the sensitivity of a pest population that is reflected in the repeated failure of a product to achieve the expected level of control when used according to the label recommendation for that pest species”. (IRAC)

Cross-resistance occurs when resistance to one insecticide confers resistance to another insecticide via the same biochemical mechanism. This can happen within insecticide chemical groups or between insecticide chemical groups. Cross-resistance may occur even if the resistant insect has never been exposed to one of the chemical classes of insecticide.

Two of the major mechanisms for neonicotinoid resistance include:—
(i) Target site resistance, whereby resistance is associated with replacement of one or more amino acids in the insecticide target protein (i.e. the nicotinic acetylcholine receptor); and
(ii) Metabolic resistance, such as enhanced oxidative detoxification of neonicotinoids due to overexpression of monooxygenases;

The cytochrome P450 monooxygenases are an important metabolic system involved in the detoxification/activation of xenobiotics. As such, P450 monooxygenases play an important role in insecticide resistance. P450 monooxygenases have such a phenomenal array of metabolizable substrates because of the presence of numerous P450s (60-111) in each species, as well as the broad substrate specificity of some P450s. Studies of monooxygenase-mediated resistance have indicated that resistance can be due to increased expression of one P450 (via increased transcription) involved in detoxification of the insecticide and might also be due to a change in the structural gene itself. As such, metabolic cross-resistance mechanisms affect not only insecticides from the given class (e.g. neonicotinoids) but also seemingly unrelated insecticides. For example, cross-resistance relationships between the neonicotinoids and pymetrozine in Bemisia tabaci have been reported by Gorman et al (Pest Management Science 2010, p. 1186-1190).

It has now been surprisingly found that compounds of formula I can be successfully used to control neonicotinoid resistant populations of insects in the Hemiptera order.

Thus, in the second aspect of the invention there is provided a method of controlling insects from the order Hemiptera which are resistant to one or more of the neonicotinoid insecticides, which method comprises applying to said neonicotinoid resistant insects a compound of formula (I).

Surprisingly, compounds of formula (I) are able to control insects that are resistant to neonicotinoid insecticides whereby resistance is a result of either of the aforementioned mechanisms (target site or metabolic).

Further, it has also been surprisingly found that compounds of formula (I) possess an advantageous safety profile with respect to beneficial arthropods, in particular beneficial insects & predatory mites.

Beneficial arthropods form a key component in integrated pest management systems. Such systems have the advantage that they are able to reduce the use of chemical agents, which provides many subsequent environmental and economic benefits & advantages. A variety of arthropods can be present whereby a grower may wish to eliminate one or more arthropod pests using a chemical insecticide whilst minimising the impact on the population of beneficial arthropods in the immediate area. However, the fact that beneficial arthropods share certain biological similarities with agricultural arthropod pests presents a significant challenge. Arthropod pests attack a plant by biting, chewing, sucking, or burrowing into the plant tissue, whereas a beneficial arthropod will most typically only use a plant as a physical support. Nevertheless, beneficial arthropods are exposed to the same environmental conditions (including chemical agents, such as insecticides) as their pest counterparts. One group of arthropods that have more intimate contact with plant materials, and which are of significant benefit to growers, are pollinators (such as honeybees). Accordingly, there is a need for new methods, compounds and compositions for controlling insects whereby undesired insects are affected but beneficial arthropods are not.

Thus, in a third aspect of the invention there is provided a method of controlling insects whereby undesired insects are affected but beneficial arthropods are not affected, which method comprises applying to the insects a compound of formula (I).

In a further aspect of the invention there is provided a method of controlling insects from the order Hemiptera which are resistant to one or more of the neonicotinoid insecticides and whereby undesired insects are affected but beneficial arthropods are not affected, which method comprises applying to said neonicotinoid resistant insects a compound of formula (I).

The compounds of formula (I) can be applied in combination with beneficial arthropods, in particular beneficial insects & predatory mites. This has the advantage that lower rates of the compounds of formula (I) can be applied to effectively control the target pest. Beneficial arthropods are useful in the control of a variety of pest species. Onus bugs in particular feed on inter alia aphids and whiteflies.

Thus, in a yet further aspect of the invention there is provided a method of controlling insects from the order Hemiptera which are resistant to one or more of the neonicotinoid insecticides, which method comprises applying to said neonicotinoid resistant insects a compound of formula (I) and one or more beneficial arthropods.

Preferred beneficial arthropods are beneficial insects & predatory mites. More preferably, Orius insidiosus, Orius laevigatus, Orius majusculus, Coccinella septempunctata, Adalia bipunctata, Amblydromalus limonicus, Amblyseius andersoni, Amblyseius barkeri, Amblyseius califomicus, Amblyseius cucumeris, Amblyseius montdorensis, Amblyseius swirskii, Phytoseiulus persimilis, Syrphus spp., or Phytoseiulus persimilis. The most preferred being Orius laevigatus.

Preferably, the neonicotinoid resistant insects from the Hemiptera order which are controlled by the methods according to the present invention are insects from suborder Sternorrhyncha, especially insects from the Aleyrodidae family and the Aphididae family.

By virtue of the surprising ability of a compound of formula I to control such neonicotinoid resistant insects, the invention also provides a method of protecting a crop of useful plants, wherein said crop is susceptible to and/or under attack from such insects. Such a method involves applying to said crop, treating a plant propagation material of said crop with, and/or applying to said insects, a compound of formula I.

Since the compounds of formula I do not exhibit cross-resistance to neonicotinoid resistant Hemiptera, it may be used in a resistance management strategy with a view to controlling resistance to the neonicotinoid class of insecticides. Such a strategy may involve alternating applications of a compound of formula I and a neonicotinoid insecticide, either on an application by application alternation (including different types of application, such as treatment of plant propagation material and foliar spray), or seasonal/crop alternation basis (e.g. use a compound of formula I on a first crop/for control in a first growing season, and use a neonicotinoid insecticide for a subsequent crop/growing season, or vice versa), and this forms yet a further aspect of the invention.

As mentioned herein, not only are insects from the Hemiptera order pests of a number of commercially important crops, the viruses that these insects carry also pose a threat. With the emergence of resistance to neonicotinoid insecticides, the severity of this threat has increased. Thus, a further aspect of the invention provides a method of controlling a plant virus in a crop of useful plants susceptible to and/or under attack by neonicotinoid resistant insects which carry said plant virus, which method comprises applying to said crop, treating a plant propagation material of said crop with, and/or applying to said insects, a compound of formula I.

Examples of plant viruses that may be controlled according to this aspect of the invention include Sobemovirus, Caulimovirus (Caulimoviridae), Closterovirus (Closteroviridae), Sequivirus (Sequiviridae), Enamovirus (Luteoviridae), Luteovirus (Luteoviridae), Polerovirus (Luteoviridae), Umbravirus, Nanovirus (Nanoviridae), Cytorhabdovirus (Rhabdoviridae), Nucleorhabdovirus (Rhabdoviridae).

These viruses are spread preferably by insects which are one or more of as an example Acyrthosiphum pisum, Aphis citricola, Aphis craccivora, Aphis fabae, Aphis frangulae, Aphis glycines, Aphis gossypii, Aphis nasturtii, Aphis pomi, Aphis spiraecola, Aulacorthum solani, Brachycaudus helichrysi, Brevicoryne brassicae, Diuraphis noxia, Dysaphis devecta, Dysaphis plantaginea, Eriosoma lanigerum, Hyalopterus pruni, Lipaphis erysimi, Macrosiphum avenae, Macrosiphum euphorbiae, Macrosiphum rosae, Myzus cerasi F., Myzus nicotianae, Myzus persicae, Nasonovia ribisnigri, Pemphigus bursarius, Phorodon humuli, Rhopalosiphum insertum Wa, Rhopalosiphum maidis Fitch, Rhopalosiphum padi L., Schizaphis graminum Rond., Sitobion avenae, Toxoptera aurantii, Toxoptera citricola, Phylloxera vitifoliae, Bemisia tabaci, Myzus persicae, Nilaparvata lugens, Aphis gossypii, Trialeurodes vaporariorum, Bactericera cockerelli.

Methods of the invention as described herein may also involve a step of assessing whether insects are resistant to neonicotinoid insecticides and/or whether said insects carry a plant virus. This step will in general involve collecting a sample of insects from the area (e.g. crop, field, habitat) to be treated, before actually applying a compound of formula I, and testing (for example using any suitable phenotypic, biochemical or molecular biological technique applicable) for resistance/sensitivity and/or the presence or absence of a virus.

The term neonicotinoid insecticide as used herein refers to any insecticidal compound that acts at the insect nicotinic acetylcholine receptor, and in particular refers to those compounds classified as neonicotinoid insectides according to Yamamoto (1996, Agrochem Jpn 68:14-15). Examples of neonicotinoid insecticides include those in Group 4A and 4C of the IRAC (insecticide resistance action committee, Crop Life) mode of action classification scheme, e.g. acetamiprid, clothianidin, dinotefuran, imidacloprid, nitenpyram, thiacloprid, sulfoxaflor and thiamethoxam, as well as any compound having the same mode of action.

By the terms “control” or “controlling” as applied to insects, it is meant that the targeted insects are repelled from or less attracted to the crops to be protected.

Additionally, as applied to insects, the terms “control” or “controlling” may also refer to the inability, or reduced ability, of the insects to feed or lay eggs. These terms may further include that the targeted insects are killed.

Thus the method of the invention may involve the use of an amount of the active ingredient that is sufficient to repel insects (i.e a repellently effective amount of active ingredient), an amount of the active ingredient that is sufficient to stop insects feeding, or it may involve the use of an insecticidally effective amount of active ingredient (i.e. an amount sufficient to kill insects), or any combination of the above effects. Where the terms “control” or “controlling” are applied to viruses it is meant that the level of viral infection of a crop of useful plants is lower than would be observed in the absence of any application of a compound of formula I.

The terms “applying” and “application” are understood to mean direct application to the insect to be controlled, as well as indirect application to said insect, for example through application to the crop or plant on which the insect acts as pest, or to the locus of said crop or insect, or indeed through treatment of the plant propagation material of said crop of plant.

Thus a compound of formula I may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the plant propagation material, such as seed, before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

Pesticidal agents or compound referred to herein using their common name are known, for example, from “The Pesticide Manual”, 15th Ed., British Crop Protection Council 2009.

The term “beneficial” arthropod or insect as used herein refers to any arthropod or insect which has at least one life stage which has a negative impact on arthropod or insect agricultural pests and/or which pollinate crop plants. The term specifically includes arthropods classed as so-called parasitoids due to their tendency to lay eggs on or in an arthropod host. Thus beneficials include pollinators, parasitoids and predators, examples include but are not limited to: Cryptolaemus montrouzieri, Encarsia formosa, Eretmocerus eremicus, Eretmocerus mundus, Feltiella acarisuga Macrophus pygmeus, Nesidiocoris tenuis, aphid midge, centipedes, ground beetles such as Pterostichus melanarius, Agonum dorsale, and Nebria brevicollis, lady beetles such as Adalia bipunctata and Coccinella septempunctata, lacewings such as Chrysoperia carnea, hoverflies such as Syrphus spp., Phytoseiulus persimilis, pirate bugs such as Orius insidiosus, Orius laevigatus, Orius majusculus, predatory mites such as Amblydromalus limonicus, Amblyseius andersoni, Amblyseius barkeri, Amblyseius californicus, Amblyseius cucumeris, Amblyseius montdorensis, Amblyseius swirskii, Phytoseiulus persimilis, predatory midges such as Aphidoletes aphidimyza, rove beetle, tachnid flies, and wasps such as Dacnusa sibirica, Diglyphus isaea Trichogramma brassicae as well as ichneumonid wasps, chalcid wasps and braconid wasps such as Aphidius colemani, Aphidius ervi, Aphidius matrcariae.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The methods of the invention are particularly applicable to the control of neonicotinoid resistant insects (and neonicotinoid resistance in insects) of the order Hemiptera, such as: Acyrthosiphum pisum, Aphis citricola, Aphis craccivora, Aphis fabae, Aphis frangulae, Aphis glycines, Aphis gossypii, Aphis nasturtii, Aphis pomi, Aphis spiraecola, Aulacorthum solani, Brachycaudus helichrysi, Brevicoryne brassicae, Diuraphis noxia, Dysaphis devecta, Dysaphis plantaginea, Eriosoma lanigerum, Hyalopterus pruni, Lipaphis erysimi, Macrosiphum avenae, Macrosiphum euphorbiae, Macrosiphum rosae, Myzus cerasi F., Myzus nicotianae, Myzus persicae, Nasonovia ribisnigri, Pemphigus bursarius, Phorodon humuli, Rhopalosiphum insertum Wa, Rhopalosiphum maidis Fitch, Rhopalosiphum padi L., Schizaphis graminum Rond., Sitobion avenae, Toxoptera aurantii, Toxoptera citricola, Phylloxera vitifoliae, Acyrthosiphon dirhodum, Acyrthosiphon solani, Aphis forbesi, Aphis grossulariae, Aphis idaei, Aphis illinoisensis, Aphis maidiradicis, Aphis ruborum, Aphis schneideri, Brachycaudus persicaecola, Cavariella aegopodii Scop., Cryptomyzus galeopsidis, Cryptomyzus ribis, Hyadaphis pseudobrassicae, Hyalopterus amygdali, Hyperomyzus pallidus, Macrosiphoniella sanborni, Metopolophium dirhodum, Myzus malisuctus, Myzus varians, Neotoxoptera sp, Nippolachnus piri Mats., Oregma lanigera Zehnter, Rhopalosiphum fitchii Sand., Rhopalosiphum nymphaeae, Rhopalosiphum sacchari Ze, Sappaphis piricola Okam.+T, Schizaphis piricola, Toxoptera theobromae Sch, and Phylloxera coccinea, Aleurodicus dispersus, Aleurocanthus spiniferus, Aleurocanthus woglumi, Aleurodicus cocois, Aleurodicus destructor, Aleurolobus barodensis, Aleurothrixus floccosus, Bemisia tabaci, Bemisia argentifolli, Dialeurodes citri, Dialeurodes citrifolli, Parabemisia myricae, Trialeurodes packardi, Trialeurodes ricini, Trialeurodes vaporariorum, Trialeurodes variabilis, Agonoscena targionii, Bactericera cockerelli, Cacopsylla pyri, Cacopsylla pyricola, Cacopsylla pyrisuga, Diaphorina citri, Glycaspis brimblecombei, Paratrioza cockerelli, Troza erytreae, Amarasca biguttula biguttula, Amritodus atkinsoni, Cicadella viridis, Cicadulina mbila, Cofana spectra, Dalbulus maidis, Empoasca decedens, Empoasca biguttula, Empoasca fabae, Empoasca vitis, Empoasca papaya, Idioscopus clypealis, Jacobiasca lybica, Laodelphax striatellus, Myndus crudus, Nephotettix virescens, Nephotettix cincticeps, Nilaparvata lugens, Peregrinus maidis, Perkinsiella saccharicida, Perkinsiella vastatrix, Recilia dorsalis, Sogatella furcifera, Tarophagus Proserpina, Zygina flammigera, Acanthocoris scabrator, Adelphocoris lineolatus, Amblypelta nitida, Bathycoelia thalassina, Blissus leucopterus, Clavigralla tomentosicollis, Edessa meditabunda, Eurydema pulchrum, Eurydema rugosum, Eurygaster Maura, Euschistus servus, Euschistus tristigmus, Euschistus heros Helopeltis antonii, Horcias nobilellus, Leptocorisa acuta, Lygus lineolaris, Lygus hesperus, Murgantia histrionic, Nesidiocoris tenuis, Nezara viridula, Oebalus insularis, Scotinophara coarctata,

Specific examples of neonicotinoid resistant Hemiptera include Bemisia tabaci, Myzus persicae, Nilaparvata lugens, Aphis gossypii, Trialeurodes vaporariorum, Bactericera cockerelli.

Preferably, the neonicotinoid resistant insects are one or more of as an example Acyrthosiphum pisum, Aphis citricola, Aphis craccivora, Aphis fabae, Aphis frangulae, Aphis glycines, Aphis gossypii, Aphis nasturtii, Aphis pomi, Aphis spiraecola, Aulacorthum solani, Brachycaudus helichrysi, Brevicoryne brassicae, Diuraphis noxia, Dysaphis devecta, Dysaphis plantaginea, Eriosoma lanigerum, Hyalopterus pruni, Lipaphis erysimi, Macrosiphum avenae, Macrosiphum euphorbiae, Macrosiphum rosae, Myzus cerasi F., Myzus nicotianae, Myzus persicae, Nasonovia ribisnigri, Pemphigus bursarius, Phorodon humuli, Rhopalosiphum insertum Wa, Rhopalosiphum maidis Fitch, Rhopalosiphum padi L., Schizaphis graminum Rond., Sitobion avenae, Toxoptera aurantii, Toxoptera citricola, Phylloxera vitifoliae, Bemisia tabaci, Myzus persicae, Nilaparvata lugens, Aphis gossypii, Trialeurodes vaporariorum, Bactericera cockerelli.

More preferably, the neonicotinoid resistant insects are one or more of as an example Bemisia tabaci, Myzus persicae, Nilaparvata lugens, Aphis gossypii, Trialeurodes vaporariorum, Bactericera cockerelli.

Most preferably the neonicotinoid resistant insects are Bemisia tabaci or Myzus persicae.

Since the methods of the invention have the effect of controlling insect pest and or viral infestation in crops of useful plants, said methods may also be viewed as methods of improving and/or maintaining plant health in said crops or as methods of increasing/maintaining the well-being of a crop.

Crops of useful plants in which the composition according to the invention can be used include perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp and jute; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria, especially those of the genus Bacillus. Further examples of toxins which can be expressed include δ-endotoxins, vegetative insecticidal proteins (Vip), insecticidal proteins of bacteria colonising nematodes, and toxins produced by scorpions, arachnids, wasps and fungi.

Example crops include: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Crops are to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The term “plant” or “useful plants” as used herein includes seedlings, bushes and trees. The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II®, (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S. A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S. A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S. A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®.

Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavour).

The table below lists key aphids (as an example of a family of Hemiptera) and crops they target.

| PEST | COMMON NAME | EXAMPLES OF CROPS |
|---|---|---|
| Acyrthosiphum pisum | Pea aphid | pea |
| Aphis citricola | Citrus aphid | citrus |
| Aphis craccivora | Cowpea aphid | vegetables, beans, sugarbeet |
| Aphis fabae | Black bean aphid | vegetables, beans, sugarbeet |
| Aphis frangulae | Breaking buckthorn | cotton potato |
| | aphid | |
| Aphis glycines | Soybean aphid | soybean |
| Aphis gossypii | Cotton aphid | cotton, vegetables, citrus, |
| | | potato |
| Aphis nasturtii | Buckthorn aphid | potato |
| Aphis pomi | Green apple aphid | apple |
| Aphis spiraecola | Green citurs aphis | apple, citrus, papaya |
| Aulacorthum solani | Foxglove aphid | citrus, sugar beet |
| Brachycaudus | Plum aphid | peach, stone fruits |
| helichrysi | | |
| Brevicoryne brassicae | Cabbage aphid | brassica |
| Diuraphis noxia | Russion wheat aphid | cereals |
| Dysaphis devecta | Leaf-curling aphid | pome fruits |
| Dysaphis plantaginea | Rosy apple aphid | pome fruits, stone fruits |
| Eriosoma lanigerum | Wooly apple aphid | pome fruits, stone fruits |
| Hyalopterus pruni | Mealy plum aphid | stone fruits |
| Lipaphis erysimi | False cabbage aphid | brassica |
| Macrosiphum avenae | Grain aphid | cereals |
| Macrosiphum | Potato aphid | potato, sugar beet, |
| euphorbiae | | vegetables |
| Macrosiphum rosae | Rose aphid | ornamentals |
| Myzus cerasi F. | Black cherry aphid | cherry, stone fruits |
| Myzus nicotianae | Tobacco aphid | tobacco |
| Myzus persicae | Peach aphid | peach, deciduous fruits, |
| | | vegetables, sugarbeet, |
| | | potato, cereals, sugarcane, |
| | | maize, ornamentals |
| Myzus persicae | Green peach aphid | peach, deciduous fruits, |
| | | vegetables, sugarbeet, |
| | | potato, cereals, sugarcane, |
| | | maize, ornamentals |
| Nasonovia ribisnigri | Lettuce aphid | vegetables |
| Pemphigus bursarius | Lettuce root aphid | vegetables |
| Phorodon humuli | Hop aphid | hops |
| Rhopalosiphum | Apple-grass aphid | Deciduous fruits, |
| insertum Wa | | ornamentals |
| Rhopalosiphum maidis | Corn leaf aphid | Maize, cereals |
| Fitch | | |
| Rhopalosiphum padi L. | Wheat aphid | Maize, cereals |
| Schizaphis graminum | Spring grain aphid | cereals |
| Rond. | | |
| Sitobion avenae | Wheat aphid | cereals |
| Toxoptera aurantii | Citrus aphid | citrus |
| Toxoptera citricola | Black citrus aphid | citrus |
| Phylloxera vitifoliae | Grape Phylloxera | vine |

The table below lists key whitefly and crops they target.

| PEST | COMMON NAME | EXAMPLES OF CROPS |
|---|---|---|
| Aleurocanthus | Orange spiney | Citrus |
| spiniferus | whitefly | |
| Aleurocanthus | Citrus blackfly | Citrus, Coffee |
| woglumi | | |
| Aleurodicus cocois | Coconut whitefly | Coconut, Cashew |
| Aleurodicus | Coconut whitefly | Coconut, Pepper |
| destructor | | |
| Aleurodicus | Spiralling whitefly | Citrus, Coconut, Soybean, |
| disperses | | Cassava, Stone Fruit, Coffee, |
| | | vegetables |
| Aleurothrixus | Wooly whitefly | Citrus, Mango, Coffee |
| floccosus | | |
| Bemisia tabaci | Tobacco whitefly | Vegetables, Cotton, Crucifera, |
| | Silverleaf whitefly | Legunes, Soyabean, Tobacco, |
| | | Potato. |
| Dialeurodes citri | Citrus whitelfy | Citrus |
| Parabemisia | Bayberry whitefly | Citrus, vegetables |
| myricae | | |
| Trialeurodes | Glasshouse | Melon, vegetables, Legumes, |
| vaporariorum | whitefly | Roses |

The table below lists key planthoppers and crops they target.

| PEST | COMMON NAME | EXAMPLES OF CROPS |
|---|---|---|
| Laodelphax | Small brown | Rice |
| striatellus | planthopper | |
| Nilaparvata lugens | Brown | Rice |
| | planthopper | |
| Sogatella furcifera | White backed | Rice |
| | planthopper | |

Accordingly, as used herein, part of a plant includes propagation material. There may be mentioned, e.g., the seeds (in the strict sense), roots, fruits, tubers, bulbs, rhizomes, parts of plants. Germinated plants and young plants, which are to be transplanted after germination or after emergence from the soil, may also be mentioned. These young plants may be protected before transplantation by a total or partial treatment by immersion.

Parts of plant and plant organs that grow at later point in time are any sections of a plant that develop from a plant propagation material, such as a seed. Parts of plant, plant organs, and plants can also benefit from the pest damage protection achieved by the application of the compound on to the plant propagation material. In an embodiment, certain parts of a plant and certain plant organs that grow at later point in time can also be considered as plant propagation material, which can themselves be applied (or treated) with the compound; and consequently, the plant, further parts of the plant and further plant organs that develop from the treated parts of plant and treated plant organs can also benefit from the pest damage protection achieved by the application of the compound on to the certain parts of plant and certain plant organs.

Methods for applying or treating pesticidal active ingredients on to plant propagation material, especially seeds, are known in the art, and include dressing, coating, pelleting and soaking application methods of the propagation material. It is preferred that the plant propagation material is a seed.

Although it is believed that the present method can be applied to a seed in any physiological state, it is preferred that the seed be in a sufficiently durable state that it incurs no damage during the treatment process. Typically, the seed would be a seed that had been harvested from the field; removed from the plant; and separated from any cob, stalk, outer husk, and surrounding pulp or other non-seed plant material. The seed would preferably also be biologically stable to the extent that the treatment would cause no biological damage to the seed. It is believed that the treatment can be applied to the seed at any time between harvest of the seed and sowing of the seed or during the sowing process (seed directed applications). The seed may also be primed either before or after the treatment.

Even distribution of the compound and adherence thereof to the seeds is desired during propagation material treatment. Treatment could vary from a thin film (dressing) of a formulation containing the compound, for example, a mixture of active ingredient(s), on a plant propagation material, such as a seed, where the original size and/or shape are recognizable to an intermediary state (such as a coating) and then to a thicker film (such as pelleting with many layers of different materials (such as carriers, for example, clays; different formulations, such as of other active ingredients; polymers; and colourants) where the original shape and/or size of the seed is no longer recognisable into the controlled release material or applied between layers of materials, or both.

The seed treatment occurs to an unsown seed, and the term “unsown seed” is meant to include seed at any period between the harvest of the seed and the sowing of the seed in the ground for the purpose of germination and growth of the plant.

Treatment to an unsown seed is not meant to include those practices in which the active ingredient is applied to the soil but would include any application practice that would target the seed during the planting process.

Preferably, the treatment occurs before sowing of the seed so that the sown seed has been pre-treated with the compound. In particular, seed coating or seed pelleting are preferred in the treatment of the compound. As a result of the treatment, the compound is adhered on to the seed and therefore available for pest control.

The treated seeds can be stored, handled, sowed and tilled in the same manner as any other active ingredient treated seed.

The compounds of formula (I) may exist in different geometric or optical isomers or tautomeric forms. This invention covers all such isomers and tautomers and mixtures thereof in all proportions as well as isotopic forms such as deuterated compounds. The invention also covers salts and N-oxides.

The compounds of the invention may contain one or more asymmetric carbon atoms, and may exist as enantiomers (or as pairs of diastereoisomers) or as mixtures of such. It is, however, preferred that a cis relative stereochemical configuration exists between the “Q” group and the “A” group of the central core structure.

Where a group has more than one substituent the substituents may be the same or different.

Alkyl groups (either alone or as part of a larger group, such as alkoxy-, alkylthio-, alkylsulfinyl-, alkylsulfonyl-, alkylcarbonyl- or alkoxycarbonyl-) can be in the form of a straight or branched chain and are, for example, methyl, ethyl, propyl, prop-2-yl, butyl, but-2-yl, 2-methyl-prop-1-yl or 2-methyl-prop-2-yl. The alkyl groups are preferably C₁-C₆, more preferably C₁-C₄, most preferably C₁-C₃ alkyl groups. Where an alkyl moiety is said to be substituted, the alkyl moiety is preferably substituted by one to four substituents, most preferably by one to three substituents.

Alkylene groups can be in the form of a straight or branched chain and are, for example, —CH₂—, —CH₂—CH₂—, —CH(CH₃)—, —CH₂—CH₂—CH₂—, —CH(CH₃)—CH₂—, or —CH(CH₂CH₃)—. The alkylene groups are preferably C₁-C₃, more preferably C₁-C₂, most preferably C₁ alkylene groups.

Alkenyl groups can be in the form of straight or branched chains, and can be, where appropriate, of either the (E)- or (Z)-configuration. Examples are vinyl and allyl. The alkenyl groups are preferably C₂-C₆, more preferably C₂-C₄, most preferably C₂-C₃ alkenyl groups.

Alkynyl groups can be in the form of straight or branched chains. Examples are ethynyl and propargyl. The alkynyl groups are preferably C₂-C₆, more preferably C₂-C₅, most preferably C₂-C₄ alkynyl groups.

Halogen is fluorine, chlorine, bromine or iodine.

Haloalkyl groups (either alone or as part of a larger group, such as haloalkoxy-, haloalkylthio-, haloalkylsulfinyl- or haloalkylsulfonyl-) are alkyl groups which are substituted by one or more of the same or different halogen atoms and are, for example, difluoromethyl, trifluoromethyl, chlorodifluoromethyl or 2,2,2-trifluoro-ethyl.

Haloalkenyl groups are alkenyl groups which are substituted by one or more of the same or different halogen atoms and are, for example, 2,2-difluoro-vinyl or 1,2-dichloro-2-fluoro-vinyl.

Haloalkynyl groups are alkynyl groups which are substituted by one or more of the same or different halogen atoms and are, for example, 1-chloro-prop-2-ynyl.

Cycloalkyl groups or carbocyclic rings can be in mono- or bi-cyclic form and are, for example, cyclopropyl, cyclobutyl, cyclohexyl and bicyclo[2.2.1]heptan-2-yl. The cycloalkyl groups are preferably C₃-C₈, more preferably C₃-C₆ cycloalkyl groups. Where a cycloalkyl moiety is said to be substituted, the cycloalkyl moiety is preferably substituted by one to four substituents, most preferably by one to three substituents.

Aryl groups (either alone or as part of a larger group, such as aryloxy) are aromatic ring systems which can be in mono-, bi- or tricyclic form. Examples of such rings include phenyl, naphthyl, anthracenyl, indenyl or phenanthrenyl. Preferred aryl groups are phenyl and naphthyl, phenyl being most preferred. Where an aryl moiety is said to be substituted, the aryl moiety is preferably substituted by one to four substituents, most preferably by one to three substituents.

Heteroaryl groups (either alone or as part of a larger group, such as heteroaryl-alkylene-) are aromatic ring systems containing at least one heteroatom and consisting either of a single ring or of two or more fused rings. Preferably, single rings will contain up to three heteroatoms and bicyclic systems up to four heteroatoms which will preferably be chosen from nitrogen, oxygen and sulfur. Examples of monocyclic groups include pyridyl, pyridazinyl, pyrimidinyl, pyrazinyl, pyrrolyl, pyrazolyl, imidazolyl, triazolyl (e.g. [1,2,4] triazolyl), furanyl, thiophenyl, oxazolyl, isoxazolyl, oxadiazolyl, thiazolyl, isothiazolyl and thiadiazolyl. Examples of bicyclic groups include purinyl, quinolinyl, cinnolinyl, quinoxalinyl, indolyl, indazolyl, benzimidazolyl, benzothiophenyl and benzothiazolyl. Monocyclic heteroaryl groups are preferred, pyridyl being most preferred. Where a heteroaryl moiety is said to be substituted, the heteroaryl moiety is preferably substituted by one to four substituents, most preferably by one to three substituents.

Heterocyclyl groups or heterocyclic rings (either alone or as part of a larger group, such as heterocyclyl-alkyl) are non-aromatic ring structures containing up to 10 atoms including one or more (preferably one, two or three) heteroatoms selected from O, S and N. Examples of monocyclic groups include, oxetanyl, 4,5-dihydro-isoxazolyl, thietanyl, pyrrolidinyl, tetrahydrofuranyl, [1,3]dioxolanyl, piperidinyl, piperazinyl, [1,4]dioxanyl, imidazolidinyl, [1,3,5]oxadiazinanyl, hexahydro-pyrimidinyl, [1,3,5]triazinanyl and morpholinyl or their oxidised versions such as 1-oxo-thietanyl and 1,1-dioxo-thietanyl. Examples of bicyclic groups include 2,3-dihydro-benzofuranyl, benzo[1,4]dioxolanyl, benzo[1,3]dioxolanyl, chromenyl, and 2,3-dihydro-benzo[1,4]dioxinyl. Where a heterocyclyl moiety is said to be substituted, the heterocyclyl moiety is preferably substituted by one to four substituents, most preferably by one to three substituents.

Preferred values of Q, A, R¹ and R² are, in any combination, as set out below.

Preferably Q is —C(═S)NR³R⁴ or —C(═NR⁵)SR⁶; where R³ and R⁴ are each independently selected from hydrogen, C₁-C₆alkyl (optionally substituted by phenyl which can be optionally substituted by one to three substituents independently selected from halogen, cyano, nitro, C₁-C₄alkyl, C₁-C₄haloalkyl, and C₁-C₄alkoxy), and C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl); and R⁵ and R⁶ are each independently selected from hydrogen, C₁-C₆alkyl (optionally substituted by phenyl which can be optionally substituted by one to three substituents independently selected from halogen, cyano, nitro, C₁-C₄alkyl, C₁-C₄haloalkyl, and C₁-C₄alkoxy), and C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl).

More preferably Q is —C(═S)NR³R⁴ or —C(═NR⁵)SR⁶; where R³ and R⁴ are each independently hydrogen or C₁-C₆alkyl; R⁵ is hydrogen; and R⁶ is C₁-C₆alkyl.

Most preferably Q is —C(═S)NR³R⁴ where R³ and R⁴ are both hydrogen.

Preferably A is —CH₂—CH₂—.

Preferably R¹ is halogen, cyano, C₁-C₃alkoxy, C₃-C₅cycloalkyl, or —C≡CR⁷ where R⁷ is hydrogen, C₁-C₄alkyl, C₃-C₅cycloalkyl (which is optionally substituted by one to two substituents independently selected from halogen, methyl and C₁-C₂haloalkyl), or tri(C₁-C₂)alkylsilyl.

More preferably R¹ is chloro, bromo, cyano, or —C≡CR⁷ where R⁷ is hydrogen.

Most preferably R¹ is chloro, bromo, or cyano.

Preferably R² is hydrogen, C₁-C₆alkyl [optionally substituted by phenyl, phenoxy, heteroaryl (wherein the heteroaryl is pyrimidinyl, pyrazolyl, imidazolyl, thiophenyl, oxazolyl, isoxazolyl, oxadiazolyl, thiazolyl, or thiadiazolyl) or heterocyclyl (wherein heterocyclyl is oxetanyl, thietanyl, tetrahydrofuranyl, [1,3]dioxolanyl, 1-oxo-thietanyl or 1,1-dioxo-thietanyl), which themselves can be optionally substituted by one to two substituents independently selected from halogen, cyano, nitro, C₁-C₄alkyl, C₁-C₄haloalkyl, and C₁-C₄alkoxyl, C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₆alkoxycarbonyl(C₁-C₆)alkyl, hydroxycarbonyl(C₁-C₆)alkyl, C₁-C₄alkylaminocarbonyl(C₁-C₆)alkyl, C₁-C₄haloalkylaminocarbonyl(C₁-C₆)alkyl, C₂-C₆alkyloxycarbonyl(C₁-C₆)alkyl, C₃-C₆cycloalkyl (optionally substituted by one to two substituents independently selected from C₁-C₂alkyl, C₁-C₂haloalkyl, and C₁-C₂alkoxy and, additionally, wherein one of the ring member units can optionally represent C═O), C₃-C₆halocycloalkyl, C₃-C₆cycloalkenyl (wherein one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, C₃-C₆alkynyl, C₃-C₆haloalkynyl, C₁-C₆alkoxycarbonyl (optionally substituted by halogen, hydroxy, cyano, C₁-C₄alkoxy, C₁-C₄haloalkyl, or phenyl), C₃-C₆alkenyloxycarbonyl, C₁-C₆alkylcarbonyl, C₁-C₆haloalkylcarbonyl, C₁-C₄alkylsulfonyl, C₁-C₄haloalkylsulfonyl, phenyl-S(═O)n² (optionally substituted by one or two substituents independently selected from halogen, nitro, and C₁-C₄alkyl) where n² is 2, heterocyclyl (wherein the heterocyclyl is oxetanyl, thietanyl, tetrahydrofuranyl, tetrahydropyranyl, [1,3]dioxolanyl, 1-oxo-thietanyl or 1,1-dioxo-thietanyl, and wherein the heterocyclyl can be optionally substituted by one to three substituents independently selected from halogen, cyano, C₁-C₂alkyl, C₁-C₂haloalkyl, C₁-C₂alkoxy, and C₁-C₂haloalkoxy, and, additionally, wherein a ring member unit can optionally represent C═O), or C₁-C₄alkyl-S(═O)n³(═NR¹⁷)—C₁-C₄alkyl wherein R¹⁷ is hydrogen, cyano, nitro, C₁-C₄alkyl and n³ is 0 or 1.

More preferably R² is hydrogen, C₁-C₄alkyl, C₁-C₆haloalkyl, C₁-C₄cyanoalkyl, C₁-C₄alkoxy(C₁-C₄)alkyl, C₁-C₂alkylcarbonyl(C₁-C₂)alkyl, C₁-C₃alkoxycarbonyl(C₁-C₃)alkyl, hydroxycarbonyl(C₁-C₃)alkyl, C₁-C₃alkylaminocarbonyl(C₁-C₃)alkyl, C₁-C₃haloalkylaminocarbonyl(C₁-C₃)alkyl, C₂-C₄alkenyloxycarbonyl(C₁-C₃)alkyl, C₃-C₆cycloalkyl, C₁-C₄alkyl-S(═O)n¹(C₁-C₄)alkyl where n¹ is 0, 1 or 2, C₃-C₆alkenyl, C₃-C₆haloalkenyl, C₃-C₆alkynyl, or heterocyclyl (wherein the heterocyclyl is oxetanyl, thietanyl, tetrahydropyranyl, 1-oxo-thietanyl or 1,1-dioxo-thietanyl).

Even more preferably R² is C₂-C₄haloalkyl, C₁-C₂alkoxy(C₂-C₃)alkyl, C₁-C₂alkoxycarbonyl(C₁-C₂)alkyl, C₃-C₄cycloalkyl, C₁-C₂alkyl-S(═O)n¹(C₂-C₃)alkyl where n¹ is 0, 1 or 2, C₃-C₆alkenyl, C₃-C₄haloalkenyl, C₃-C₄alkynyl, or heterocyclyl (wherein heterocyclyl is oxetanyl, thietanyl, 1-oxo-thietanyl or 1,1-dioxo-thietanyl).

Most preferably R² is C₂-C₄haloalkyl, C₁-C₂alkoxy(C₂-C₃)alkyl, C₃-C₄cycloalkyl, C₁-C₂alkyl-S(═O)n¹(C₂alkyl) where n¹ is 0, C₃-C₄alkenyl, or propargyl.

Embodiments according to the invention are provided as set out below.

Embodiment 1 provides compounds of formula I, or an agrochemically acceptable salt, N-oxide or isomer thereof, as defined above.

Embodiment 2 provides compounds, or an agrochemically acceptable salt, N-oxide or isomer thereof, according to embodiment 1 wherein Q is —C(═S)NR³R⁴ or —C(═NR⁵)SR⁶; where R³ and R⁴ are each independently selected from hydrogen, C₁-C₆alkyl (optionally substituted by phenyl which can be optionally substituted by one to three substituents independently selected from halogen, cyano, nitro, C₁-C₄alkyl, C₁-C₄haloalkyl, and C₁-C₄alkoxy), and C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₆alkenyl); and R⁵ and R⁶ are each independently selected from hydrogen, C₁-C₆alkyl (optionally substituted by phenyl which can be optionally substituted by one to three substituents independently selected from halogen, cyano, nitro, C₁-C₄alkyl, C₁-C₄haloalkyl, and C₁-C₄alkoxy), and C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl).

Embodiment 3 provides compounds, or an agrochemically acceptable salt, N-oxide or isomer thereof, according to embodiment 1 or 2 wherein A is —CH₂—CH₂—. Embodiment 4 provides compounds, or an agrochemically acceptable salt, N-oxide or isomer thereof, according to embodiment 1, 2 or 3 wherein R¹ is halogen, cyano, C₁-C₃alkoxy, C₃-C₅cycloalkyl, or —C≡CR⁷ where R⁷ is hydrogen, C₁-C₄alkyl, C₃-C₅cycloalkyl (which is optionally substituted by one to two substituents independently selected from halogen, methyl and C₁-C₂haloalkyl), or tri(C₁-C₂)alkylsilyl.

Embodiment 5 provides compounds, or an agrochemically acceptable salt, N-oxide or isomer thereof, according to embodiment 1, 2, 3 or 4 wherein R² is hydrogen, C₁-C₆alkyl [optionally substituted by phenyl, phenoxy, heteroaryl (wherein the heteroaryl is pyrimidinyl, pyrazolyl, imidazolyl, thiophenyl, oxazolyl, isoxazolyl, oxadiazolyl, thiazolyl, or thiadiazolyl) or heterocyclyl (wherein heterocyclyl is oxetanyl, thietanyl, tetrahydrofuranyl, [1,3]dioxolanyl, 1-oxo-thietanyl or 1,1-dioxo-thietanyl), which themselves can be optionally substituted by one to two substituents independently selected from halogen, cyano, nitro, C₁-C₄alkyl, C₁-C₄haloalkyl, and C₁-C₄alkoxyl, C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₆alkoxycarbonyl(C₁-C₆)alkyl, hydroxycarbonyl(C₁-C₆)alkyl, C₁-C₄alkylaminocarbonyl(C₁-C₆)alkyl, C₁-C₄haloalkylaminocarbonyl(C₁-C₆)alkyl, C₂-C₆alkenyloxycarbonyl(C₁-C₆)alkyl, C₃-C₆cycloalkyl (optionally substituted by one to two substituents independently selected from C₁-C₂alkyl, C₁-C₂haloalkyl, and C₁-C₂alkoxy and, additionally, wherein one of the ring member units can optionally represent C═O), C₃-C₆halocycloalkyl, C₃-C₆cycloalkenyl (wherein one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, C₃-C₆alkynyl, C₃-C₆haloalkynyl, C₁-C₆alkoxycarbonyl (optionally substituted by halogen, hydroxy, cyano, C₁-C₄alkoxy, C₁-C₄haloalkyl, or phenyl), C₃-C₆alkenyloxycarbonyl, C₁-C₆alkylcarbonyl, C₁-C₆haloalkylcarbonyl, C₁-C₄alkylsulfonyl, C₁-C₄haloalkylsulfonyl, phenyl-S(═O)n² (optionally substituted by one or two substituents independently selected from halogen, nitro, and C₁-C₄alkyl) where n² is 2, heterocyclyl (wherein the heterocyclyl is oxetanyl, thietanyl, tetrahydrofuranyl, tetrahydropyranyl, [1,3]dioxolanyl, 1-oxo-thietanyl or 1,1-dioxo-thietanyl, and wherein the heterocyclyl can be optionally substituted by one to three substituents independently selected from halogen, cyano, C₁-C₂alkyl, C₁-C₂haloalkyl, C₁-C₂alkoxy, and C₁-C₂haloalkoxy, and, additionally, wherein a ring member unit can optionally represent C═O), or C₁-C₄alkyl-S(═O)n³(═NR¹⁷)—C₁-C₄alkyl wherein R¹⁷ is hydrogen, cyano, nitro, C₁-C₄alkyl and n³ is 0 or 1.

Embodiment 6 provides compounds, or an agrochemically acceptable salt, N-oxide or isomer thereof, according to any one of embodiments 1, 2, 3, 4, or 5 wherein Q is —C(═S)NR³R⁴ or —C(═NR⁵)SR⁶; where R³ and R⁴ are each independently hydrogen or C₁-C₆alkyl; R⁵ is hydrogen; and R⁶ is C₁-C₆alkyl.

Embodiment 7 provides compounds, or an agrochemically acceptable salt, N-oxide or isomer thereof, according to any one of embodiments 1, 2, 3, 4, 5, or 6 wherein R¹ is chloro, bromo, cyano, or —C≡CR⁷ where R⁷ is hydrogen.

Embodiment 8 provides compounds, or an agrochemically acceptable salt, N-oxide or isomer thereof, according to any one of embodiments 1, 2, 3, 4, 5, 6, or 7 wherein R² is hydrogen, C₁-C₄alkyl, C₁-C₆haloalkyl, C₁-C₄cyanoalkyl, C₁-C₄alkoxy(C₁-C₄)alkyl, C₁-C₂alkylcarbonyl(C₁-C₂)alkyl, C₁-C₃alkoxycarbonyl(C₁-C₃)alkyl, hydroxycarbonyl(C₁-C₃)alkyl, C₁-C₃alkylaminocarbonyl(C₁-C₃)alkyl, C₁-C₃haloalkylaminocarbonyl(C₁-C₃)alkyl, C₂-C₄alkenyloxycarbonyl(C₁-C₃)alkyl, C₃-C₆cycloalkyl, C₁-C₄alkyl-S(═O)n¹(C₁-C₄)alkyl where n¹ is 0, 1 or 2, C₃-C₆alkenyl, C₃-C₆haloalkenyl, C₃-C₆alkynyl, or heterocyclyl (wherein the heterocyclyl is oxetanyl, thietanyl, tetrahydropyranyl, 1-oxo-thietanyl or 1,1-dioxo-thietanyl).

Embodiment 9 provides compounds, or an agrochemically acceptable salt, N-oxide or isomer thereof, according to any one of embodiments 1, 2, 3, 4, 5, 6, 7, or 8 wherein Q is —C(═S)NR³R⁴ where R³ and R⁴ are both hydrogen.

Embodiment 10 provides compounds, or an agrochemically acceptable salt, N-oxide or isomer thereof, according to any one of embodiments 1, 2, 3, 4, 5, 6, 7, 8, or 9 wherein R¹ is chloro, bromo, or cyano.

Embodiment 11 provides compounds, or an agrochemically acceptable salt, N-oxide or isomer thereof, according to any one of embodiments 1, 2, 3, 4, 5, 6, 7, 8, 9 or 10 wherein R² is C₂-C₄haloalkyl, C₁-C₂alkoxy(C₂-C₃)alkyl, C₁-C₂alkoxycarbonyl(C₁-C₂)alkyl, C₃-C₄cycloalkyl, C₁-C₂alkyl-S(═O)n¹(C₂-C₃)alkyl where n¹ is 0, 1 or 2, C₃-C₆alkenyl, C₃-C₄haloalkenyl, C₃-C₄alkynyl, or heterocyclyl (wherein heterocyclyl is oxetanyl, thietanyl, 1-oxo-thietanyl or 1,1-dioxo-thietanyl).

Embodiment 12 provides compounds, or an agrochemically acceptable salt, N-oxide or isomer thereof, according to any one of embodiments 1, 2, 3, 4, 5, 6, 7, 8, 9, 10 or 11 wherein R² is C₂-C₄haloalkyl, C₁-C₂alkoxy(C₂-C₃)alkyl, C₃-C₄cycloalkyl, C₁-C₂alkyl-S(═O)n¹(C₂alkyl) where n¹ is 0, C₃-C₄alkenyl, or propargyl.

A preferred group of compounds are those of formula (Ia) which are compounds of formula (I) wherein Q is —C(═S)NR³R⁴ or —C(═NR⁵)SR⁶; where R³ and R⁴ are each independently selected from hydrogen, C₁-C₆alkyl (optionally substituted by phenyl which can be optionally substituted by one to three substituents independently selected from halogen, cyano, nitro, C₁-C₄alkyl, C₁-C₄haloalkyl, and C₁-C₄alkoxy), and C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl); and R⁵ and R⁶ are each independently selected from hydrogen, C₁-C₆alkyl (optionally substituted by phenyl which can be optionally substituted by one to three substituents independently selected from halogen, cyano, nitro, C₁-C₄alkyl, C₁-C₄haloalkyl, and C₁-C₄alkoxy), and C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl); R¹ is halogen, cyano, C₁-C₃alkoxy, C₃-C₅cycloalkyl, or —C≡CR⁷ where R⁷ is hydrogen, C₁-C₄alkyl, C₃-C₅cycloalkyl (which is optionally substituted by one to two substituents independently selected from halogen, methyl and C₁-C₂haloalkyl), or tri(C₁-C₂)alkylsilyl; and R² is hydrogen, C₁-C₆alkyl [optionally substituted by phenyl, phenoxy, heteroaryl (wherein the heteroaryl is pyrimidinyl, pyrazolyl, imidazolyl, thiophenyl, oxazolyl, isoxazolyl, oxadiazolyl, thiazolyl, or thiadiazolyl) or heterocyclyl (wherein heterocyclyl is oxetanyl, thietanyl, tetrahydrofuranyl, [1,3]dioxolanyl, 1-oxo-thietanyl or 1,1-dioxo-thietanyl), which themselves can be optionally substituted by one to two substituents independently selected from halogen, cyano, nitro, C₁-C₄alkyl, C₁-C₄haloalkyl, and C₁-C₄alkoxyl, C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₆alkoxycarbonyl(C₁-C₆)alkyl, hydroxycarbonyl(C₁-C₆)alkyl, C₁-C₄alkylaminocarbonyl(C₁-C₆)alkyl, C₁-C₄haloalkylaminocarbonyl(C₁-C₆)alkyl, C₂-C₆alkenyloxycarbonyl(C₁-C₆)alkyl, C₃-C₆cycloalkyl (optionally substituted by one to two substituents independently selected from C₁-C₂alkyl, C₁-C₂haloalkyl, and C₁-C₂alkoxy and, additionally, wherein one of the ring member units can optionally represent C═O), C₃-C₆halocycloalkyl, C₃-C₆cycloalkenyl (wherein one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, C₃-C₆alkynyl, C₃-C₆haloalkynyl, C₁-C₆alkoxycarbonyl (optionally substituted by halogen, hydroxy, cyano, C₁-C₄alkoxy, C₁-C₄haloalkyl, or phenyl), C₃-C₆alkenyloxycarbonyl, C₁-C₆alkylcarbonyl, C₁-C₆haloalkylcarbonyl, C₁-C₄alkylsulfonyl, C₁-C₄haloalkylsulfonyl, phenyl-S(═O)n² (optionally substituted by one or two substituents independently selected from halogen, nitro, and C₁-C₄alkyl) where n² is 2, heterocyclyl (wherein the heterocyclyl is oxetanyl, thietanyl, tetrahydrofuranyl, tetrahydropyranyl, [1,3]dioxolanyl, 1-oxo-thietanyl or 1,1-dioxo-thietanyl, and wherein the heterocyclyl can be optionally substituted by one to three substituents independently selected from halogen, cyano, C₁-C₂alkyl, C₁-C₂haloalkyl, C₁-C₂alkoxy, and C₁-C₂haloalkoxy, and, additionally, wherein a ring member unit can optionally represent C═O), or C₁-C₄alkyl-S(═O)n³(═NR¹⁷)—C₁-C₄alkyl wherein R¹⁷ is hydrogen, cyano, nitro, C₁-C₄alkyl and n³ is 0 or 1; or an agrochemically acceptable salt, N-oxide or isomer thereof.

A preferred group of compounds of formula (Ia) are compounds of formula (Iaa) wherein R¹ is chloro, bromo, cyano, or —C≡CR⁷ where R⁷ is hydrogen.

A preferred group of compounds of formula (Iaa) are compounds of formula (Iaaa) wherein R¹ is chloro, bromo, or cyano.

Another preferred group of compounds of formula (Ia) are compounds of formula (lab) wherein R² is hydrogen, C₁-C₄alkyl, C₁-C₆haloalkyl, C₁-C₄cyanoalkyl, C₁-C₄alkoxy(C₁-C₄)alkyl, C₁-C₂alkylcarbonyl(C₁-C₂)alkyl, C₁-C₃alkoxycarbonyl(C₁-C₃)alkyl, hydroxycarbonyl(C₁-C₃)alkyl, C₁-C₃alkylaminocarbonyl(C₁-C₃)alkyl, C₁-C₃haloalkylaminocarbonyl(C₁-C₃)alkyl, C₂-C₄alkenyloxycarbonyl(C₁-C₃)alkyl, C₃-C₆cycloalkyl, C₁-C₄alkyl-S(═O)n¹(C₁-C₄)alkyl where n¹ is 0, 1 or 2, C₃-C₆alkenyl, C₃-C₆haloalkenyl, C₃-C₆alkynyl, or heterocyclyl (wherein the heterocyclyl is oxetanyl, thietanyl, tetrahydropyranyl, 1-oxo-thietanyl or 1,1-dioxo-thietanyl).

A preferred group of compounds of formula (lab) are compounds of formula (Iaba) wherein R² is C₂-C₄haloalkyl, C₁-C₂alkoxy(C₂-C₃)alkyl, C₁-C₂alkoxycarbonyl(C₁-C₂)alkyl, C₃-C₄cycloalkyl, C₁-C₂alkyl-S(═O)n¹(C₂-C₃)alkyl where n¹ is 0, 1 or 2, C₃-C₅alkenyl, C₃-C₄haloalkenyl, C₃-C₄alkynyl, or heterocyclyl (wherein heterocyclyl is oxetanyl, thietanyl, 1-oxo-thietanyl or 1,1-dioxo-thietanyl).

A preferred group of compounds of formula (Iaba) are compounds of formula (Iabaa) wherein R² is C₂-C₄haloalkyl, C₁-C₂alkoxy(C₂-C₃)alkyl, C₃-C₄cycloalkyl, C₁-C₂alkyl-S(═O)n¹(C₂alkyl) where n¹ is 0, C₃-C₄alkenyl, or propargyl.

Another preferred group of compounds are those of formula (Ib) which are compounds of formula (I) wherein Q is —C(═S)NR³R⁴ or —C(═NR⁵)SR⁶; where R³ and R⁴ are each independently hydrogen or C₁-C₆alkyl; R⁵ is hydrogen; and R⁶ is C₁-C₆alkyl; R¹ is halogen, cyano, C₁-C₃alkoxy, C₃-C₅cycloalkyl, or —C≡CR⁷ where R⁷ is hydrogen, C₁-C₄alkyl, C₃-C₅cycloalkyl (which is optionally substituted by one to two substituents independently selected from halogen, methyl and C₁-C₂haloalkyl), or tri(C₁-C₂)alkylsilyl; and R² is hydrogen, C₁-C₆alkyl [optionally substituted by phenyl, phenoxy, heteroaryl (wherein the heteroaryl is pyrimidinyl, pyrazolyl, imidazolyl, thiophenyl, oxazolyl, isoxazolyl, oxadiazolyl, thiazolyl, or thiadiazolyl) or heterocyclyl (wherein heterocyclyl is oxetanyl, thietanyl, tetrahydrofuranyl, [1,3]dioxolanyl, 1-oxo-thietanyl or 1,1-dioxo-thietanyl), which themselves can be optionally substituted by one to two substituents independently selected from halogen, cyano, nitro, C₁-C₄alkyl, C₁-C₄haloalkyl, and C₁-C₄alkoxy], C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₆alkoxycarbonyl(C₁-C₆)alkyl, hydroxycarbonyl(C₁-C₆)alkyl, C₁-C₄alkylaminocarbonyl(C₁-C₆)alkyl, C₁-C₄haloalkylaminocarbonyl(C₁-C₆)alkyl, C₂-C₆alkyloxycarbonyl(C₁-C₆)alkyl, C₃-C₆cycloalkyl (optionally substituted by one to two substituents independently selected from C₁-C₂alkyl, C₁-C₂haloalkyl, and C₁-C₂alkoxy and, additionally, wherein one of the ring member units can optionally represent C═O), C₃-C₆halocycloalkyl, C₃-C₆cycloalkenyl (wherein one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, C₃-C₆alkynyl, C₃-C₆haloalkynyl, C₁-C₆alkoxycarbonyl (optionally substituted by halogen, hydroxy, cyano, C₁-C₄alkoxy, C₁-C₄haloalkyl, or phenyl), C₃-C₆alkenyloxycarbonyl, C₁-C₆alkylcarbonyl, C₁-C₆haloalkylcarbonyl, C₁-C₄alkylsulfonyl, C₁-C₄haloalkylsulfonyl, phenyl-S(═O)n² (optionally substituted by one or two substituents independently selected from halogen, nitro, and C₁-C₄alkyl) where n² is 2, heterocyclyl (wherein the heterocyclyl is oxetanyl, thietanyl, tetrahydrofuranyl, tetrahydropyranyl, [1,3]dioxolanyl, 1-oxo-thietanyl or 1,1-dioxo-thietanyl, and wherein the heterocyclyl can be optionally substituted by one to three substituents independently selected from halogen, cyano, C₁-C₂alkyl, C₁-C₂haloalkyl, C₁-C₂alkoxy, and C₁-C₂haloalkoxy, and, additionally, wherein a ring member unit can optionally represent C═O), or C₁-C₄alkyl-S(═O)n³(═NR¹⁷)—C₁-C₄alkyl wherein R¹⁷ is hydrogen, cyano, nitro, C₁-C₄alkyl and n³ is 0 or 1; or an agrochemically acceptable salt, N-oxide or isomer thereof.

A preferred group of compounds of formula (Ib) are compounds of formula (Iba) wherein R¹ is chloro, bromo, cyano, or —C≡CR⁷ where R⁷ is hydrogen.

A preferred group of compounds of formula (Iba) are compounds of formula (Ibaa) wherein R¹ is chloro, bromo, or cyano.

Another preferred group of compounds of formula (Ib) are compounds of formula (Ibb) wherein R² is hydrogen, C₁-C₄alkyl, C₁-C₆haloalkyl, C₁-C₄cyanoalkyl, C₁-C₄alkoxy(C₁-C₄)alkyl, C₁-C₂alkylcarbonyl(C₁-C₂)alkyl, C₁-C₃alkoxycarbonyl(C₁-C₃)alkyl, hydroxycarbonyl(C₁-C₃)alkyl, C₁-C₃alkylaminocarbonyl(C₁-C₃)alkyl, C₁-C₃haloalkylaminocarbonyl(C₁-C₃)alkyl, C₂-C₄alkenyloxycarbonyl(C₁-C₃)alkyl, C₃-C₆cycloalkyl, C₁-C₄alkyl-S(═O)n¹(C₁-C₄)alkyl where n¹ is 0, 1 or 2, C₃-C₆alkenyl, C₃-C₆haloalkenyl, C₃-C₆alkynyl, or heterocyclyl (wherein the heterocyclyl is oxetanyl, thietanyl, tetrahydropyranyl, 1-oxo-thietanyl or 1,1-dioxo-thietanyl).

A preferred group of compounds of formula (Ibb) are compounds of formula (Ibba) wherein R² is C₂-C₄haloalkyl, C₁-C₂alkoxy(C₂-C₃)alkyl, C₁-C₂alkoxycarbonyl(C₁-C₂)alkyl, C₃-C₄cycloalkyl, C₁-C₂alkyl-S(═O)n¹(C₂-C₃)alkyl where n¹ is 0, 1 or 2, C₃-C₅alkenyl, C₃-C₄haloalkenyl, C₃-C₄alkynyl, or heterocyclyl (wherein heterocyclyl is oxetanyl, thietanyl, 1-oxo-thietanyl or 1,1-dioxo-thietanyl).

A preferred group of compounds of formula (Ibba) are compounds of formula (Ibbaa) wherein R² is C₂-C₄haloalkyl, C₁-C₂alkoxy(C₂-C₃)alkyl, C₃-C₄cycloalkyl, C₁-C₂alkyl-S(═O)n¹(C₂alkyl) where n¹ is 0, C₃-C₄alkenyl, or propargyl.

Another preferred group of compounds are those of formula (Ic) which are compounds of formula (I) wherein Q is —C(═S)NR³R⁴ where R³ and R⁴ are both hydrogen; R¹ is halogen, cyano, C₁-C₃alkoxy, C₃-C₅cycloalkyl, or —C≡CR⁷ where R⁷ is hydrogen, C₁-C₄alkyl, C₃-C₅cycloalkyl (which is optionally substituted by one to two substituents independently selected from halogen, methyl and C₁-C₂haloalkyl), or tri(C₁-C₂)alkylsilyl; and R² is hydrogen, C₁-C₆alkyl [optionally substituted by phenyl, phenoxy, heteroaryl (wherein the heteroaryl is pyrimidinyl, pyrazolyl, imidazolyl, thiophenyl, oxazolyl, isoxazolyl, oxadiazolyl, thiazolyl, or thiadiazolyl) or heterocyclyl (wherein heterocyclyl is oxetanyl, thietanyl, tetrahydrofuranyl, [1,3]dioxolanyl, 1-oxo-thietanyl or 1,1-dioxo-thietanyl), which themselves can be optionally substituted by one to two substituents independently selected from halogen, cyano, nitro, C₁-C₄alkyl, C₁-C₄haloalkyl, and C₁-C₄alkoxy], C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₆alkoxycarbonyl(C₁-C₆)alkyl, hydroxycarbonyl(C₁-C₆)alkyl, C₁-C₄alkylaminocarbonyl(C₁-C₆)alkyl, C₁-C₄haloalkylaminocarbonyl(C₁-C₆)alkyl, C₂-C₆alkenyloxycarbonyl(C₁-C₆)alkyl, C₃-C₆cycloalkyl (optionally substituted by one to two substituents independently selected from C₁-C₂alkyl, C₁-C₂haloalkyl, and C₁-C₂alkoxy and, additionally, wherein one of the ring member units can optionally represent C═O), C₃-C₆halocycloalkyl, C₃-C₆cycloalkenyl (wherein one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, C₃-C₆alkynyl, C₃-C₆haloalkynyl, C₁-C₆alkoxycarbonyl (optionally substituted by halogen, hydroxy, cyano, C₁-C₄alkoxy, C₁-C₄haloalkyl, or phenyl), C₃-C₆alkenyloxycarbonyl, C₁-C₆alkylcarbonyl, C₁-C₆haloalkylcarbonyl, C₁-C₄alkylsulfonyl, C₁-C₄haloalkylsulfonyl, phenyl-S(═O)n² (optionally substituted by one or two substituents independently selected from halogen, nitro, and C₁-C₄alkyl) where n² is 2, heterocyclyl (wherein the heterocyclyl is oxetanyl, thietanyl, tetrahydrofuranyl, tetrahydropyranyl, [1,3]dioxolanyl, 1-oxo-thietanyl or 1,1-dioxo-thietanyl, and wherein the heterocyclyl can be optionally substituted by one to three substituents independently selected from halogen, cyano, C₁-C₂alkyl, C₁-C₂haloalkyl, C₁-C₂alkoxy, and C₁-C₂haloalkoxy, and, additionally, wherein a ring member unit can optionally represent C═O), or C₁-C₄alkyl-S(═O)n³(═NR¹⁷)—C₁-C₄alkyl wherein R¹⁷ is hydrogen, cyano, nitro, C₁-C₄alkyl and n³ is 0 or 1; or an agrochemically acceptable salt, N-oxide or isomer thereof.

A preferred group of compounds of formula (Ic) are compounds of formula (Ica) wherein R¹ is chloro, bromo, cyano, or —C≡CR⁷ where R⁷ is hydrogen.

A preferred group of compounds of formula (Ica) are compounds of formula (Icaa) wherein R¹ is chloro, bromo, or cyano.

Another preferred group of compounds of formula (Ic) are compounds of formula (Icb) wherein R² is hydrogen, C₁-C₄alkyl, C₁-C₆haloalkyl, C₁-C₄cyanoalkyl, C₁-C₄alkoxy(C₁-C₄)alkyl, C₁-C₂alkylcarbonyl(C₁-C₂)alkyl, C₁-C₃alkoxycarbonyl(C₁-C₃)alkyl, hydroxycarbonyl(C₁-C₃)alkyl, C₁-C₃alkylaminocarbonyl(C₁-C₃)alkyl, C₁-C₃haloalkylaminocarbonyl(C₁-C₃)alkyl, C₂-C₄alkenyloxycarbonyl(C₁-C₃)alkyl, C₃-C₆cycloalkyl, C₁-C₄alkyl-S(═O)n¹(C₁-C₄)alkyl where n¹ is 0, 1 or 2, C₃-C₆alkenyl, C₃-C₆haloalkenyl, C₃-C₆alkynyl, or heterocyclyl (wherein the heterocyclyl is oxetanyl, thietanyl, tetrahydropyranyl, 1-oxo-thietanyl or 1,1-dioxo-thietanyl).

A preferred group of compounds of formula (Icb) are compounds of formula (Icba) wherein R² is C₂-C₄haloalkyl, C₁-C₂alkoxy(C₂-C₃)alkyl, C₁-C₂alkoxycarbonyl(C₁-C₂)alkyl, C₃-C₄cycloalkyl, C₁-C₂alkyl-S(═O)n¹(C₂-C₃)alkyl where n¹ is 0, 1 or 2, C₃-C₆alkenyl, C₃-C₄haloalkenyl, C₃-C₄alkynyl, or heterocyclyl (wherein heterocyclyl is oxetanyl, thietanyl, 1-oxo-thietanyl or 1,1-dioxo-thietanyl).

A preferred group of compounds of formula (Icba) are compounds of formula (ICBAA) wherein R² is C₂-C₄haloalkyl, C₁-C₂alkoxy(C₂-C₃)alkyl, C₃-C₄cycloalkyl, C₁-C₂alkyl-S(═O)n¹(C₂alkyl) where n¹ is 0, C₃-C₄alkenyl, or propargyl.

A particularly preferred group of compounds are those of formula (Id) which are compounds of formula (I) wherein Q is —C(═S)NR³R⁴ or —C(═NR⁵)SR⁶; where R³ and R⁴ are each independently hydrogen or C₁-C₆alkyl; R⁵ is hydrogen; and R⁶ is C₁-C₆alkyl; R¹ is chloro, bromo, cyano, or —C≡CR⁷ where R⁷ is hydrogen; and R² is hydrogen, C₁-C₄alkyl, C₁-C₆haloalkyl, C₁-C₄cyanoalkyl, C₁-C₄alkoxy(C₁-C₄)alkyl, C₁-C₂alkylcarbonyl(C₁-C₂)alkyl, C₁-C₃alkoxycarbonyl(C₁-C₃)alkyl, hydroxycarbonyl(C₁-C₃)alkyl, C₁-C₃alkylaminocarbonyl(C₁-C₃)alkyl, C₁-C₃haloalkylaminocarbonyl(C₁-C₃)alkyl, C₂-C₄alkenyloxycarbonyl(C₁-C₃)alkyl, C₃-C₆cycloalkyl, C₁-C₄alkyl-S(═O)n¹(C₁-C₄)alkyl where n¹ is 0, 1 or 2, C₃-C₆alkenyl, C₃-C₆haloalkenyl, C₃-C₆alkynyl, or heterocyclyl (wherein the heterocyclyl is oxetanyl, thietanyl, tetrahydropyranyl, 1-oxo-thietanyl or 1,1-dioxo-thietanyl); or an agrochemically acceptable salt, N-oxide or isomer thereof.

A preferred group of compounds of formula (Id) are compounds of formula (IDA) wherein R² is C₂-C₄haloalkyl, C₁-C₂alkoxy(C₂-C₃)alkyl, C₁-C₂alkoxycarbonyl(C₁-C₂)alkyl, C₃-C₄cycloalkyl, C₁-C₂alkyl-S(═O)n¹(C₂-C₃)alkyl where n¹ is 0, 1 or 2, C₃-C₅alkenyl, C₃-C₄haloalkenyl, C₃-C₄alkynyl, or heterocyclyl (wherein heterocyclyl is oxetanyl, thietanyl, 1-oxo-thietanyl or 1,1-dioxo-thietanyl).

A most preferred group of compounds are those of formula (Ie) which are compounds of formula (I) wherein Q is —C(═S)NR³R⁴ where R³ and R⁴ are both hydrogen; R¹ is chloro, bromo, or cyano; and R² is C₂-C₄haloalkyl, C₁-C₂alkoxy(C₂-C₃)alkyl, C₃-C₄cycloalkyl, C₁-C₂alkyl-S(═O)n¹(C₂alkyl) where n¹ is 0, C₃-C₄alkenyl, or propargyl; or an agrochemically acceptable salt, N-oxide or isomer thereof.

The tables below illustrate specific compounds of the invention.

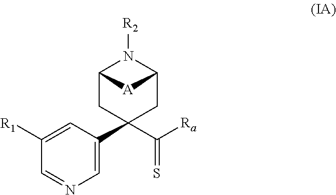

TABLE P1

| Entry | R¹ | A | R^{a} |
|---|---|---|---|
| 1 | Cl | —CH₂—CH₂— | NH₂ |
| 2 | Br | —CH₂—CH₂— | NH₂ |
| 3 | CN | —CH₂—CH₂— | NH₂ |
| 4 | ethynyl | —CH₂—CH₂— | NH₂ |
| 5 | F | —CH₂—CH₂— | NH₂ |
| 6 | I | —CH₂—CH₂— | NH₂ |
| 7 | prop-1-ynyl | —CH₂—CH₂— | NH₂ |
| 8 | cyclopropyl | —CH₂—CH₂— | NH₂ |
| 9 | MeO | —CH₂—CH₂— | NH₂ |
| 10 | EtO | —CH₂—CH₂— | NH₂ |
| 11 | Cl | —CH═CH— | NH₂ |
| 12 | Br | —CH═CH— | NH₂ |
| 13 | CN | —CH═CH— | NH₂ |
| 14 | ethynyl | —CH═CH— | NH₂ |
| 15 | F | —CH═CH— | NH₂ |
| 16 | I | —CH═CH— | NH₂ |
| 17 | prop-1-ynyl | —CH═CH— | NH₂ |
| 18 | cyclopropyl | —CH═CH— | NH₂ |
| 19 | MeO | —CH═CH— | NH₂ |
| 20 | EtO | —CH═CH— | NH₂ |
| 21 | Cl | —CH₂—CH₂— | NHMe |
| 22 | Br | —CH₂—CH₂— | NHMe |
| 23 | CN | —CH₂—CH₂— | NHMe |
| 24 | ethynyl | —CH₂—CH₂— | NHMe |
| 25 | F | —CH₂—CH₂— | NHMe |
| 26 | I | —CH₂—CH₂— | NHMe |
| 27 | prop-1-ynyl | —CH₂—CH₂— | NHMe |
| 28 | cyclopropyl | —CH₂—CH₂— | NHMe |
| 29 | MeO | —CH₂—CH₂— | NHMe |
| 30 | EtO | —CH₂—CH₂— | NHMe |
| 31 | Cl | —CH═CH— | NHMe |
| 32 | Br | —CH═CH— | NHMe |
| 33 | CN | —CH═CH— | NHMe |
| 34 | ethynyl | —CH═CH— | NHMe |
| 35 | F | —CH═CH— | NHMe |
| 36 | I | —CH═CH— | NHMe |
| 37 | prop-1-ynyl | —CH═CH— | NHMe |
| 38 | cyclopropyl | —CH═CH— | NHMe |
| 39 | MeO | —CH═CH— | NHMe |
| 40 | EtO | —CH═CH— | NHMe |
| 41 | Cl | —CH₂—CH₂— | NMe₂ |
| 42 | Br | —CH₂—CH₂— | NMe₂ |
| 43 | CN | —CH₂—CH₂— | NMe₂ |
| 44 | ethynyl | —CH₂—CH₂— | NMe₂ |
| 45 | F | —CH₂—CH₂— | NMe₂ |
| 46 | I | —CH₂—CH₂— | NMe₂ |
| 47 | prop-1-ynyl | —CH₂—CH₂— | NMe₂ |
| 48 | cyclopropyl | —CH₂—CH₂— | NMe₂ |
| 49 | MeO | —CH₂—CH₂— | NMe₂ |
| 50 | EtO | —CH₂—CH₂— | NMe₂ |
| 51 | Cl | —CH═CH— | NMe₂ |
| 52 | Br | —CH═CH— | NMe₂ |
| 53 | CN | —CH═CH— | NMe₂ |
| 54 | ethynyl | —CH═CH— | NMe₂ |
| 55 | F | —CH═CH— | NMe₂ |
| 56 | I | —CH═CH— | NMe₂ |
| 57 | prop-1-ynyl | —CH═CH— | NMe₂ |
| 58 | cyclopropyl | —CH═CH— | NMe₂ |
| 59 | MeO | —CH═CH— | NMe₂ |
| 60 | EtO | —CH═CH— | NMe₂ |
| 61 | Cl | —CH₂—CH₂— | NHEt |
| 62 | Br | —CH₂—CH₂— | NHEt |
| 63 | CN | —CH₂—CH₂— | NHEt |
| 64 | ethynyl | —CH₂—CH₂— | NHEt |
| 65 | F | —CH₂—CH₂— | NHEt |
| 66 | I | —CH₂—CH₂— | NHEt |
| 67 | prop-1-ynyl | —CH₂—CH₂— | NHEt |
| 68 | cyclopropyl | —CH₂—CH₂— | NHEt |
| 69 | MeO | —CH₂—CH₂— | NHEt |
| 70 | EtO | —CH₂—CH₂— | NHEt |
| 71 | Cl | —CH═CH— | NHEt |
| 72 | Br | —CH═CH— | NHEt |
| 73 | CN | —CH═CH— | NHEt |
| 74 | ethynyl | —CH═CH— | NHEt |
| 75 | F | —CH═CH— | NHEt |
| 76 | I | —CH═CH— | NHEt |
| 77 | prop-1-ynyl | —CH═CH— | NHEt |
| 78 | cyclopropyl | —CH═CH— | NHEt |
| 79 | MeO | —CH═CH— | NHEt |
| 80 | EtO | —CH═CH— | NHEt |
| 81 | Cl | —CH₂—CH₂— | NEt₂ |
| 82 | Br | —CH₂—CH₂— | NEt₂ |
| 83 | CN | —CH₂—CH₂— | NEt₂ |
| 84 | ethynyl | —CH₂—CH₂— | NEt₂ |
| 85 | F | —CH₂—CH₂— | NEt₂ |
| 86 | I | —CH₂—CH₂— | NEt₂ |
| 87 | prop-1-ynyl | —CH₂—CH₂— | NEt₂ |
| 88 | cyclopropyl | —CH₂—CH₂— | NEt₂ |
| 89 | MeO | —CH₂—CH₂— | NEt₂ |
| 90 | EtO | —CH₂—CH₂— | NEt₂ |
| 91 | Cl | —CH═CH— | NEt₂ |
| 92 | Br | —CH═CH— | NEt₂ |
| 93 | CN | —CH═CH— | NEt₂ |
| 94 | ethynyl | —CH═CH— | NEt₂ |
| 95 | F | —CH═CH— | NEt₂ |
| 96 | I | —CH═CH— | NEt₂ |
| 97 | prop-1-ynyl | —CH═CH— | NEt₂ |
| 98 | cyclopropyl | —CH═CH— | NEt₂ |
| 99 | MeO | —CH═CH— | NEt₂ |
| 100 | EtO | —CH═CH— | NEt₂ |
| 101 | Cl | —CH₂—CH₂— | NHiBu |
| 102 | Br | —CH₂—CH₂— | NHiBu |
| 103 | CN | —CH₂—CH₂— | NHiBu |
| 104 | ethynyl | —CH₂—CH₂— | NHiBu |
| 105 | F | —CH₂—CH₂— | NHiBu |
| 106 | I | —CH₂—CH₂— | NHiBu |
| 107 | prop-1-ynyl | —CH₂—CH₂— | NHiBu |
| 108 | cyclopropyl | —CH₂—CH₂— | NHiBu |
| 109 | MeO | —CH₂—CH₂— | NHiBu |
| 110 | EtO | —CH₂—CH₂— | NHiBu |
| 111 | Cl | —CH═CH— | NHiBu |
| 112 | Br | —CH═CH— | NHiBu |
| 113 | CN | —CH═CH— | NHiBu |
| 114 | ethynyl | —CH═CH— | NHiBu |
| 115 | F | —CH═CH— | NHiBu |
| 116 | I | —CH═CH— | NHiBu |
| 117 | prop-1-ynyl | —CH═CH— | NHiBu |
| 118 | cyclopropyl | —CH═CH— | NHiBu |
| 119 | MeO | —CH═CH— | NHiBu |
| 120 | EtO | —CH═CH— | NHiBu |
| 121 | Cl | —CH₂—CH₂— | NHPr |
| 122 | Br | —CH₂—CH₂— | NHPr |
| 123 | CN | —CH₂—CH₂— | NHPr |
| 124 | ethynyl | —CH₂—CH₂— | NHPr |
| 125 | F | —CH₂—CH₂— | NHPr |
| 126 | I | —CH₂—CH₂— | NHPr |
| 127 | prop-1-ynyl | —CH₂—CH₂— | NHPr |
| 128 | cyclopropyl | —CH₂—CH₂— | NHPr |
| 129 | MeO | —CH₂—CH₂— | NHPr |
| 130 | EtO | —CH₂—CH₂— | NHPr |
| 131 | Cl | —CH═CH— | NHPr |
| 132 | Br | —CH═CH— | NHPr |
| 133 | CN | —CH═CH— | NHPr |
| 134 | ethynyl | —CH═CH— | NHPr |
| 135 | F | —CH═CH— | NHPr |
| 136 | I | —CH═CH— | NHPr |
| 137 | prop-1-ynyl | —CH═CH— | NHPr |
| 138 | cyclopropyl | —CH═CH— | NHPr |
| 139 | MeO | —CH═CH— | NHPr |
| 140 | EtO | —CH═CH— | NHPr |
| 141 | Cl | —CH₂—CH₂— | NHiPr |
| 142 | Br | —CH₂—CH₂— | NHiPr |
| 143 | CN | —CH₂—CH₂— | NHiPr |
| 144 | ethynyl | —CH₂—CH₂— | NHiPr |
| 145 | F | —CH₂—CH₂— | NHiPr |
| 146 | I | —CH₂—CH₂— | NHiPr |
| 147 | prop-1-ynyl | —CH₂—CH₂— | NHiPr |
| 148 | cyclopropyl | —CH₂—CH₂— | NHiPr |
| 149 | MeO | —CH₂—CH₂— | NHiPr |
| 150 | EtO | —CH₂—CH₂— | NHiPr |
| 151 | Cl | —CH═CH— | NHiPr |
| 152 | Br | —CH═CH— | NHiPr |
| 153 | CN | —CH═CH— | NHiPr |
| 154 | ethynyl | —CH═CH— | NHiPr |
| 155 | F | —CH═CH— | NHiPr |
| 156 | I | —CH═CH— | NHiPr |
| 157 | prop-1-ynyl | —CH═CH— | NHiPr |
| 158 | cyclopropyl | —CH═CH— | NHiPr |
| 159 | MeO | —CH═CH— | NHiPr |
| 160 | EtO | —CH═CH— | NHiPr |
| 161 | Cl | —CH₂—CH₂— | NHnBu |
| 162 | Br | —CH₂—CH₂— | NHnBu |
| 163 | CN | —CH₂—CH₂— | NHnBu |
| 164 | ethynyl | —CH₂—CH₂— | NHnBu |
| 165 | F | —CH₂—CH₂— | NHnBu |
| 166 | I | —CH₂—CH₂— | NHnBu |
| 167 | prop-1-ynyl | —CH₂—CH₂— | NHnBu |
| 168 | cyclopropyl | —CH₂—CH₂— | NHnBu |
| 169 | MeO | —CH₂—CH₂— | NHnBu |
| 170 | EtO | —CH₂—CH₂— | NHnBu |
| 171 | Cl | —CH═CH— | NHnBu |
| 172 | Br | —CH═CH— | NHnBu |
| 173 | CN | —CH═CH— | NHnBu |
| 174 | ethynyl | —CH═CH— | NHnBu |
| 175 | F | —CH═CH— | NHnBu |
| 176 | I | —CH═CH— | NHnBu |
| 177 | prop-1-ynyl | —CH═CH— | NHnBu |
| 178 | cyclopropyl | —CH═CH— | NHnBu |
| 179 | MeO | —CH═CH— | NHnBu |
| 180 | EtO | —CH═CH— | NHnBu |
| 181 | Cl | —CH₂—CH₂— | NHBn |
| 182 | Br | —CH₂—CH₂— | NHBn |
| 183 | CN | —CH₂—CH₂— | NHBn |
| 184 | ethynyl | —CH₂—CH₂— | NHBn |
| 185 | F | —CH₂—CH₂— | NHBn |
| 186 | I | —CH₂—CH₂— | NHBn |
| 187 | prop-1-ynyl | —CH₂—CH₂— | NHBn |
| 188 | cyclopropyl | —CH₂—CH₂— | NHBn |
| 189 | MeO | —CH₂—CH₂— | NHBn |
| 190 | EtO | —CH₂—CH₂— | NHBn |
| 191 | Cl | —CH═CH— | NHBn |
| 192 | Br | —CH═CH— | NHBn |
| 193 | CN | —CH═CH— | NHBn |
| 194 | ethynyl | —CH═CH— | NHBn |
| 195 | F | —CH═CH— | NHBn |
| 196 | I | —CH═CH— | NHBn |
| 197 | prop-1-ynyl | —CH═CH— | NHBn |
| 198 | cyclopropyl | —CH═CH— | NHBn |
| 199 | MeO | —CH═CH— | NHBn |
| 200 | EtO | —CH═CH— | NHBn |

### Table 1

Table 1 provides 200 compounds of (IA) wherein R² is (1,1-dioxothietan-3-yl)methyl, and R¹,
A, Ra are as defined in Table P1 (above).

### Table 2

Table 2 provides 200 compounds of (IA) wherein R² is (2-oxo-1,3-dioxolan-4-yl)methyl, and R¹, A, Ra are as defined in Table P1 (above).

### Table 3

Table 3 provides 200 compounds of (IA) wherein R² is (2-oxotetrahydrofuran-3-yl)methyl, and R¹, A, Ra are as defined in Table P1 (above).

### Table 4

Table 4 provides 200 compounds of (IA) wherein R² is (5-oxotetrahydrofuran-2-yl)methyl, and R¹, A, Ra are as defined in Table P1 (above).

### Table 5

Table 5 provides 200 compounds of (IA) wherein R² is (CH₂)₂S(O)₂Me, and R¹, A, Ra are as defined in Table P1 (above).

### Table 6

Table 6 provides 200 compounds of (IA) wherein R² is (CH₂)₂S(O)₂NHMe, and R¹, A, R^{a} are as defined in Table P1 (above).

### Table 7

Table 7 provides 200 compounds of (IA) wherein R² is (CH₂)₂S(O)Me, and R¹, A, Ra are as defined in Table P1 (above).

### Table 8

Table 8 provides 200 compounds of (IA) wherein R² is (E)-1-methylbut-2-enyl, and R¹, A, R^{a} are as defined in Table P1 (above).

### Table 9

Table 9 provides 200 compounds of (IA) wherein R² is (S)—CHMeC(O)OMe, and R¹, A, R^{a} are as defined in Table P1 (above).

### Table 10

Table 10 provides 200 compounds of (IA) wherein R² is 1-methylbut-2-enyl, and R¹, A, R^{a} are as defined in Table P1 (above).

### Table 11

Table 11 provides 200 compounds of (IA) wherein R² is (Z)-2,3-dichloroallyl, and R¹, A, R^{a} are as defined in Table P1 (above).

### Table 12

Table 12 provides 200 compounds of (IA) wherein R² is (Z)-3-chlorobut-2-enyl, and R¹, A, R^{a} are as defined in Table P1 (above).

### Table 13

Table 13 provides 200 compounds of (IA) wherein R² is (R)—CHMeC(O)OMe, and R¹, A, R^{a} are as defined in Table P1 (above).

### Table 14

Table 14 provides 200 compounds of (IA) wherein R² is 1,1-dimethylallyl, and R¹, A, Ra are as defined in Table P1 (above).

### Table 15

Table 15 provides 200 compounds of (IA) wherein R² is 1,1-dimethylprop-2-ynyl, and R¹, A, R^{a} are as defined in Table P1 (above).

### Table 16

Table 16 provides 200 compounds of (IA) wherein R² is 1,1-dioxothietan-3-yl, and R¹, A, R^{a} are as defined in Table P1 (above).

### Table 17

Table 17 provides 200 compounds of (IA) wherein R² is 1,3-dioxolan-2-ylmethyl, and R¹, A, R^{a} are as defined in Table P1 (above).

### Table 18

Table 18 provides 200 compounds of (IA) wherein R² is 1,3-dithian-5-yl, and R¹, A, Ra are as defined in Table P1 (above).

### Table 19

Table 19 provides 200 compounds of (IA) wherein R² is 1-cyano-1-methyl-ethyl, and R¹, A, R^{a} are as defined in Table P1 (above).

### Table 20

Table 20 provides 200 compounds of (IA) wherein R² is 1-cyano-2-methyl-propyl, and R¹, A, R^{a} are as defined in Table P1 (above).

### Table 21

Table 21 provides 200 compounds of (IA) wherein R² is 1-methoxycarbonylpropyl, and R¹, A, Ra are as defined in Table P1 (above).

### Table 22

Table 22 provides 200 compounds of (IA) wherein R² is 1-methyl-2-methylsulfanyl-ethyl, and R¹, A, Ra are as defined in Table P1 (above).

### Table 23

Table 23 provides 200 compounds of (IA) wherein R² is 1-methyl-2-oxo-2-propoxy-ethyl, and R¹, A, Ra are as defined in Table P1 (above).

### Table 24

Table 24 provides 200 compounds of (IA) wherein R² is 1-methyl-2-oxo-propyl, and R¹, A, R^{a} are as defined in Table P1 (above).

### Table 25

Table 25 provides 200 compounds of (IA) wherein R² is 1-methylallyl, and R¹, A, Ra are as defined in Table P1 (above).

### Table 26

Table 26 provides 200 compounds of (IA) wherein R² is 1-methylprop-2-ynyl, and R¹, A, R^{a} are as defined in Table P1 (above).

### Table 27

Table 27 provides 200 compounds of (IA) wherein R² is 1-oxothietan-3-yl, and R¹, A, Ra are as defined in Table P1 (above).

### Table 28

Table 28 provides 200 compounds of (IA) wherein R² is 2-(2,2-difluoroethylamino)-2-oxo-ethyl, and R¹, A, Ra are as defined in Table P1 (above).

### Table 29

Table 29 provides 200 compounds of (IA) wherein R² is CHMeC(O)OMe, and R¹, A, R^{a} are as defined in Table P1 (above).

### Table 30

Table 30 provides 200 compounds of (IA) wherein R² is 2-(methylsulfonimidoyl)ethyl, and R¹, A, Ra are as defined in Table P1 (above).

### Table 31

Table 31 provides 200 compounds of (IA) wherein R² is 2,2,2-trifluoro-1-methyl-ethyl, and R¹, A, Ra are as defined in Table P1 (above).

### Table 32

Table 32 provides 200 compounds of (IA) wherein R² is 2,2,2-trifluoroethyl, and R¹, A, Ra are as defined in Table P1 (above).

### Table 33

Table 33 provides 200 compounds of (IA) wherein R² is 2,2,3,3,3-pentafluoropropyl, and R¹, A, Ra are as defined in Table P1 (above).

### Table 34

Table 34 provides 200 compounds of (IA) wherein R² is 2,2,3,3-tetrafluoropropyl, and R¹, A, R^{a} are as defined in Table P1 (above).

### Table 35

Table 35 provides 200 compounds of (IA) wherein R² is 2,2-difluorobutyl, and R¹, A, Ra are as defined in Table P1 (above).

### Table 36

Table 36 provides 200 compounds of (IA) wherein R² is 2,2-difluoroethyl, and R¹, A, Ra are as defined in Table P1 (above).

### Table 37

Table 37 provides 200 compounds of (IA) wherein R² is 2,2-difluoropropyl, and R¹, A, Ra are as defined in Table P1 (above).

### Table 38

Table 38 provides 200 compounds of (IA) wherein R² is 2,2-dimethylbut-3-ynyl, and R¹, A, R^{a} are as defined in Table P1 (above).

### Table 39

Table 39 provides 200 compounds of (IA) wherein R² is 2-allyloxy-1-methyl-2-oxo-ethyl, and R¹, A, Ra are as defined in Table P1 (above).

### Table 40

Table 40 provides 200 compounds of (IA) wherein R² is 2-carboxy-3,3,3-trifluoro-propyl, and R¹, A, Ra are as defined in Table P1 (above).

### Table 41

Table 41 provides 200 compounds of (IA) wherein R² is 2-cyanoallyl, and R¹, A, Ra are as defined in Table P1 (above).

### Table 42

Table 42 provides 200 compounds of (IA) wherein R² is 2-cyanoethyl, and R¹, A, Ra are as defined in Table P1 (above).

### Table 43

Table 43 provides 200 compounds of (IA) wherein R² is 2-ethoxy-1-methyl-2-oxo-ethyl, and R¹, A, Ra are as defined in Table P1 (above).

### Table 44

Table 44 provides 200 compounds of (IA) wherein R² is 2-ethoxy-2-oxo-ethyl, and R¹, A, R^{a} are as defined in Table P1 (above).

### Table 45

Table 45 provides 200 compounds of (IA) wherein R² is 2-fluoroallyl, and R¹, A, Ra are as defined in Table P1 (above).

### Table 46

Table 46 provides 200 compounds of (IA) wherein R² is 2-fluoroethyl, and R¹, A, Ra are as defined in Table P1 (above).

### Table 47

Table 47 provides 200 compounds of (IA) wherein R² is 2-methoxy-1-methyl-ethyl, and R¹, A, Ra are as defined in Table P1 (above).

### Table 48

Table 48 provides 200 compounds of (IA) wherein R² is 2-methoxyethyl, and R¹, A, Ra are as defined in Table P1 (above).

### Table 49

Table 49 provides 200 compounds of (IA) wherein R² is 2-methylallyl, and R¹, A, Ra are as defined in Table P1 (above).

### Table 50

Table 50 provides 200 compounds of (IA) wherein R² is 2-methylsulfanylethyl, and R¹, A, R^{a} are as defined in Table P1 (above).

### Table 51

Table 51 provides 200 compounds of (IA) wherein R² is 2-oxobutyl, and R¹, A, Ra are as defined in Table P1 (above).

### Table 52

Table 52 provides 200 compounds of (IA) wherein R² is 3,3,3-trifluoropropyl, and R¹, A, R^{a} are as defined in Table P1 (above).

### Table 53

Table 53 provides 200 compounds of (IA) wherein R² is 3-ethoxy-3-oxo-propyl, and R¹, A, R^{a} are as defined in Table P1 (above).

### Table 54

Table 54 provides 200 compounds of (IA) wherein R² is 3-fluoropropyl, and R¹, A, Ra are as defined in Table P1 (above).

### Table 55

Table 55 provides 200 compounds of (IA) wherein R² is 3-methoxy-2-methyl-3-oxo-propanoyl, and R¹, A, R^{a} are as defined in Table P1 (above).

### Table 56

Table 56 provides 200 compounds of (IA) wherein R² is 3-methylbut-2-enyl, and R¹, A, R^{a} are as defined in Table P1 (above).

### Table 57

Table 57 provides 200 compounds of (IA) wherein R² is 3-oxocyclohexen-1-yl, and R¹, A, R^{a} are as defined in Table P1 (above).

### Table 58

Table 58 provides 200 compounds of (IA) wherein R² is 3-oxocyclopenten-1-yl, and R¹, A, R^{a} are as defined in Table P1 (above).

### Table 59

Table 59 provides 200 compounds of (IA) wherein R² is 3-t-butoxy-3-oxo-propyl, and R¹, A, R^{a} are as defined in Table P1 (above).

### Table 60

Table 60 provides 200 compounds of (IA) wherein R² is 2-chloroallyl, and R¹, A, Ra are as defined in Table P1 (above).

### Table 61

Table 61 provides 200 compounds of (IA) wherein R² is 4,4,4-trifluorobutyl, and R¹, A, Ra are as defined in Table P1 (above).

### Table 62

Table 62 provides 200 compounds of (IA) wherein R² is 4-methoxybut-2-ynyl, and R¹, A, R^{a} are as defined in Table P1 (above).

### Table 63

Table 63 provides 200 compounds of (IA) wherein R² is (5-methyl-2-oxo-1,3-dioxol-4-yl)methyl, and R¹, A, Ra are as defined in Table P1 (above).

### Table 64

Table 64 provides 200 compounds of (IA) wherein R² is acetyl, and R¹, A, R^{a} are as defined in Table P1 (above).

### Table 65

Table 65 provides 200 compounds of (IA) wherein R² is allyl, and R¹, A, Ra are as defined in Table P1 (above).

### Table 66

Table 66 provides 200 compounds of (IA) wherein R² is benzyl, and R¹, A, Ra are as defined in Table P1 (above).

### Table 67

Table 67 provides 200 compounds of (IA) wherein R² is but-2-ynyl, and R¹, A, Ra are as defined in Table P1 (above).

### Table 68

Table 68 provides 200 compounds of (IA) wherein R² is but-3-ynyl, and R¹, A, Ra are as defined in Table P1 (above).

### Table 69

Table 69 provides 200 compounds of (IA) wherein R² is C(O)OMe, and R¹, A, R^{a} are as defined in Table P1 (above).

### Table 70

Table 70 provides 200 compounds of (IA) wherein R² is C(O)Ot-Bu, and R¹, A, R^{a} are as defined in Table P1 (above).

### Table 71

Table 71 provides 200 compounds of (IA) wherein R² is CH(CO₂Et)₂, and R¹, A, Ra are as defined in Table P1 (above).

### Table 72

Table 72 provides 200 compounds of (IA) wherein R² is CH(S), and R¹, A, R^{a} are as defined in Table P1 (above).

### Table 73

Table 73 provides 200 compounds of (IA) wherein R² is CH₂C(O)Me, and R¹, A, R^{a} are as defined in Table P1 (above).

### Table 74

Table 74 provides 200 compounds of (IA) wherein R² is CH₂C(O)NHMe, and R¹, A, R^{a} are as defined in Table P1 (above).

### Table 75

Table 75 provides 200 compounds of (IA) wherein R² is CH₂C(O)OH, and R¹, A, Ra are as defined in Table P1 (above).

### Table 76

Table 76 provides 200 compounds of (IA) wherein R² is CH₂C(O)OMe, and R¹, A, R^{a} are as defined in Table P1 (above).

### Table 77

Table 77 provides 200 compounds of (IA) wherein R² is CH₂CH₂OEt, and R¹, A, Ra are as defined in Table P1 (above).

### Table 78

Table 78 provides 200 compounds of (IA) wherein R² is CH₂CN, and R¹, A, Ra are as defined in Table P1 (above).

### Table 79

Table 79 provides 200 compounds of (IA) wherein R² is CH₂S(O)₂NHMe, and R¹, A, Ra are as defined in Table P1 (above).

### Table 80

Table 80 provides 200 compounds of (IA) wherein R² is cyclobutyl, and R¹, A, Ra are as defined in Table P1 (above).

### Table 81

Table 81 provides 200 compounds of (IA) wherein R² is cyclopropyl, and R¹, A, Ra are as defined in Table P1 (above).

### Table 82

Table 82 provides 200 compounds of (IA) wherein R² is ethyl, and R¹, A, Ra are as defined in Table P1 (above).

### Table 83

Table 83 provides 200 compounds of (IA) wherein R² is formyl, and R¹, A, Ra are as defined in Table P1 (above).

### Table 84

Table 84 provides 200 compounds of (IA) wherein R² is hydrogen, and R¹, A, Ra are as defined in Table P1 (above).

### Table 85

Table 85 provides 200 compounds of (IA) wherein R² is isobutyl, and R¹, A, Ra are as defined in Table P1 (above).

### Table 86

Table 86 provides 200 compounds of (IA) wherein R² is isopropyl, and R¹, A, Ra are as defined in Table P1 (above).

### Table 87

Table 87 provides 200 compounds of (IA) wherein R² is methyl, and R¹, A, Ra are as defined in Table P1 (above).

### Table 88

Table 88 provides 200 compounds of (IA) wherein R² is n-Bu, and R¹, A, Ra are as defined in Table P1 (above).

### Table 89

Table 89 provides 200 compounds of (IA) wherein R² is n-hexyl, and R¹, A, Ra are as defined in Table P1 (above).

### Table 90

Table 90 provides 200 compounds of (IA) wherein R² is n-Pr, and R¹, A, Ra are as defined in Table P1 (above).

### Table 91

Table 91 provides 200 compounds of (IA) wherein R² is oxetan-2-ylmethyl, and R¹, A, Ra are as defined in Table P1 (above).

### Table 92

Table 92 provides 200 compounds of (IA) wherein R² is oxetan-3-yl, and R¹, A, Ra are as defined in Table P1 (above).

### Table 93

Table 93 provides 200 compounds of (IA) wherein R² is oxetan-3-ylmethyl, and R¹, A, Ra are as defined in Table P1 (above).

### Table 94

Table 94 provides 200 compounds of (IA) wherein R² is cyclobutylmethyl, and R¹, A, Ra are as defined in Table P1 (above).

### Table 95

Table 95 provides 200 compounds of (IA) wherein R² is pent-2-ynyl, and R¹, A, Ra are as defined in Table P1 (above).

### Table 96

Table 96 provides 200 compounds of (IA) wherein R² is pent-4-ynyl, and R¹, A, Ra are as defined in Table P1 (above).

### Table 97

Table 97 provides 200 compounds of (IA) wherein R² is propargyl, and R¹, A, Ra are as defined in Table P1 (above).

### Table 98

Table 98 provides 200 compounds of (IA) wherein R² is t-Bu, and R¹, A, Ra are as defined in Table P1 (above).

### Table 99

Table 99 provides 200 compounds of (IA) wherein R² is tetrahydrofuran-3-ylmethyl, and R¹, A, Ra are as defined in Table P1 (above).

### Table 100

Table 100 provides 200 compounds of (IA) wherein R² is tetrahydropyran-4-yl, and R¹, A, R^{a} are as defined in Table P1 (above).

### Table 101

Table 101 provides 200 compounds of (IA) wherein R² is tetrahydrothiophen-2-ylmethyl, and R¹, A, Ra are as defined in Table P1 (above).

### Table 102

Table 102 provides 200 compounds of (IA) wherein R² is tetrahydrothiophen-3-ylmethyl, and R¹, A, Ra are as defined in Table P1 (above).

### Table 103

Table 103 provides 200 compounds of (IA) wherein R² is thietan-3-yl, and R¹, A, Ra are as defined in Table P1 (above).

### Table 104

Table 104 provides 200 compounds of (IA) wherein R² is thietan-3-ylmethyl, and R¹, A, R^{a} are as defined in Table P1 (above).

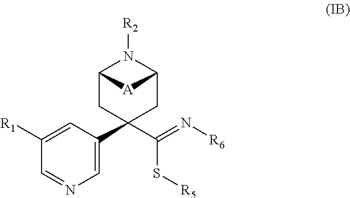

TABLE P2

| Entry | R¹ | A | R⁵ | R⁶ |
|---|---|---|---|---|
| 1 | Cl | —CH₂—CH₂— | Me | H |
| 2 | Br | —CH₂—CH₂— | Me | H |
| 3 | CN | —CH₂—CH₂— | Me | H |
| 4 | ethynyl | —CH₂—CH₂— | Me | H |
| 5 | F | —CH₂—CH₂— | Me | H |
| 6 | I | —CH₂—CH₂— | Me | H |
| 7 | prop-1-ynyl | —CH₂—CH₂— | Me | H |
| 8 | MeO | —CH₂—CH₂— | Me | H |
| 9 | EtO | —CH₂—CH₂— | Me | H |
| 10 | cyclopropyl | —CH₂—CH₂— | Me | H |
| 11 | Cl | —CH═CH— | Me | H |
| 12 | Br | —CH═CH— | Me | H |
| 13 | CN | —CH═CH— | Me | H |
| 14 | ethynyl | —CH═CH— | Me | H |
| 15 | F | —CH═CH— | Me | H |
| 16 | I | —CH═CH— | Me | H |
| 17 | prop-1-ynyl | —CH═CH— | Me | H |
| 18 | MeO | —CH═CH— | Me | H |
| 19 | EtO | —CH═CH— | Me | H |
| 20 | cyclopropyl | —CH═CH— | Me | H |
| 21 | Cl | —CH₂—CH₂— | Et | H |
| 22 | Br | —CH₂—CH₂— | Et | H |
| 23 | CN | —CH₂—CH₂— | Et | H |
| 24 | ethynyl | —CH₂—CH₂— | Et | H |
| 25 | F | —CH₂—CH₂— | Et | H |
| 26 | I | —CH₂—CH₂— | Et | H |
| 27 | prop-1-ynyl | —CH₂—CH₂— | Et | H |
| 28 | MeO | —CH₂—CH₂— | Et | H |
| 29 | EtO | —CH₂—CH₂— | Et | H |
| 30 | cyclopropyl | —CH₂—CH₂— | Et | H |
| 31 | Cl | —CH═CH— | Et | H |
| 32 | Br | —CH═CH— | Et | H |
| 33 | CN | —CH═CH— | Et | H |
| 34 | ethynyl | —CH═CH— | Et | H |
| 35 | F | —CH═CH— | Et | H |
| 36 | I | —CH═CH— | Et | H |
| 37 | prop-1-ynyl | —CH═CH— | Et | H |
| 38 | MeO | —CH═CH— | Et | H |
| 39 | EtO | —CH═CH— | Et | H |
| 40 | cyclopropyl | —CH═CH— | Et | H |
| 41 | Cl | —CH₂—CH₂— | Bn | H |
| 42 | Br | —CH₂—CH₂— | Bn | H |
| 43 | CN | —CH₂—CH₂— | Bn | H |
| 44 | ethynyl | —CH₂—CH₂— | Bn | H |
| 45 | F | —CH₂—CH₂— | Bn | H |
| 46 | I | —CH₂—CH₂— | Bn | H |
| 47 | prop-1-ynyl | —CH₂—CH₂— | Bn | H |
| 48 | MeO | —CH₂—CH₂— | Bn | H |
| 49 | EtO | —CH₂—CH₂— | Bn | H |
| 50 | cyclopropyl | —CH₂—CH₂— | Bn | H |
| 51 | Cl | —CH═CH— | Bn | H |
| 52 | Br | —CH═CH— | Bn | H |
| 53 | CN | —CH═CH— | Bn | H |
| 54 | ethynyl | —CH═CH— | Bn | H |
| 55 | F | —CH═CH— | Bn | H |
| 56 | I | —CH═CH— | Bn | H |
| 57 | prop-1-ynyl | —CH═CH— | Bn | H |
| 58 | MeO | —CH═CH— | Bn | H |
| 59 | EtO | —CH═CH— | Bn | H |
| 60 | cyclopropyl | —CH═CH— | Bn | H |
| 61 | Cl | —CH₂—CH₂— | Ph | H |
| 62 | Br | —CH₂—CH₂— | Ph | H |
| 63 | CN | —CH₂—CH₂— | Ph | H |
| 64 | ethynyl | —CH₂—CH₂— | Ph | H |
| 65 | F | —CH₂—CH₂— | Ph | H |
| 66 | I | —CH₂—CH₂— | Ph | H |
| 67 | prop-1-ynyl | —CH₂—CH₂— | Ph | H |
| 68 | MeO | —CH₂—CH₂— | Ph | H |
| 69 | EtO | —CH₂—CH₂— | Ph | H |
| 70 | cyclopropyl | —CH₂—CH₂— | Ph | H |
| 71 | Cl | —CH═CH— | Ph | H |
| 72 | Br | —CH═CH— | Ph | H |
| 73 | CN | —CH═CH— | Ph | H |
| 74 | ethynyl | —CH═CH— | Ph | H |
| 75 | F | —CH═CH— | Ph | H |
| 76 | I | —CH═CH— | Ph | H |
| 77 | prop-1-ynyl | —CH═CH— | Ph | H |
| 78 | MeO | —CH═CH— | Ph | H |
| 79 | EtO | —CH═CH— | Ph | H |
| 80 | cyclopropyl | —CH═CH— | Ph | H |
| 81 | Cl | —CH₂—CH₂— | Me | Me |
| 82 | Br | —CH₂—CH₂— | Me | Me |
| 83 | CN | —CH₂—CH₂— | Me | Me |
| 84 | ethynyl | —CH₂—CH₂— | Me | Me |
| 85 | F | —CH₂—CH₂— | Me | Me |
| 86 | I | —CH₂—CH₂— | Me | Me |
| 87 | prop-1-ynyl | —CH₂—CH₂— | Me | Me |
| 88 | MeO | —CH₂—CH₂— | Me | Me |
| 89 | EtO | —CH₂—CH₂— | Me | Me |
| 90 | cyclopropyl | —CH₂—CH₂— | Me | Me |
| 91 | Cl | —CH═CH— | Me | Me |
| 92 | Br | —CH═CH— | Me | Me |
| 93 | CN | —CH═CH— | Me | Me |
| 94 | ethynyl | —CH═CH— | Me | Me |
| 95 | F | —CH═CH— | Me | Me |
| 96 | I | —CH═CH— | Me | Me |
| 97 | prop-1-ynyl | —CH═CH— | Me | Me |
| 98 | MeO | —CH═CH— | Me | Me |
| 99 | EtO | —CH═CH— | Me | Me |
| 100 | cyclopropyl | —CH═CH— | Me | Me |
| 101 | Cl | —CH₂—CH₂— | Et | Me |
| 102 | Br | —CH₂—CH₂— | Et | Me |
| 103 | CN | —CH₂—CH₂— | Et | Me |
| 104 | ethynyl | —CH₂—CH₂— | Et | Me |
| 105 | F | —CH₂—CH₂— | Et | Me |
| 106 | I | —CH₂—CH₂— | Et | Me |
| 107 | prop-1-ynyl | —CH₂—CH₂— | Et | Me |
| 108 | MeO | —CH₂—CH₂— | Et | Me |
| 109 | EtO | —CH₂—CH₂— | Et | Me |
| 110 | cyclopropyl | —CH₂—CH₂— | Et | Me |
| 111 | Cl | —CH═CH— | Et | Me |
| 112 | Br | —CH═CH— | Et | Me |
| 113 | CN | —CH═CH— | Et | Me |
| 114 | ethynyl | —CH═CH— | Et | Me |
| 115 | F | —CH═CH— | Et | Me |
| 116 | I | —CH═CH— | Et | Me |
| 117 | prop-1-ynyl | —CH═CH— | Et | Me |
| 118 | MeO | —CH═CH— | Et | Me |
| 119 | EtO | —CH═CH— | Et | Me |
| 120 | cyclopropyl | —CH═CH— | Et | Me |
| 121 | Cl | —CH₂—CH₂— | Bn | Me |
| 122 | Br | —CH₂—CH₂— | Bn | Me |
| 123 | CN | —CH₂—CH₂— | Bn | Me |
| 124 | ethynyl | —CH₂—CH₂— | Bn | Me |
| 125 | F | —CH₂—CH₂— | Bn | Me |
| 126 | I | —CH₂—CH₂— | Bn | Me |
| 127 | prop-1-ynyl | —CH₂—CH₂— | Bn | Me |
| 128 | MeO | —CH₂—CH₂— | Bn | Me |
| 129 | EtO | —CH₂—CH₂— | Bn | Me |
| 130 | cyclopropyl | —CH₂—CH₂— | Bn | Me |
| 131 | Cl | —CH═CH— | Bn | Me |
| 132 | Br | —CH═CH— | Bn | Me |
| 133 | CN | —CH═CH— | Bn | Me |
| 134 | ethynyl | —CH═CH— | Bn | Me |
| 135 | F | —CH═CH— | Bn | Me |
| 136 | I | —CH═CH— | Bn | Me |
| 137 | prop-1-ynyl | —CH═CH— | Bn | Me |
| 138 | MeO | —CH═CH— | Bn | Me |
| 139 | EtO | —CH═CH— | Bn | Me |
| 140 | cyclopropyl | —CH═CH— | Bn | Me |
| 141 | Cl | —CH₂—CH₂— | Ph | Me |
| 142 | Br | —CH₂—CH₂— | Ph | Me |
| 143 | CN | —CH₂—CH₂— | Ph | Me |
| 144 | ethynyl | —CH₂—CH₂— | Ph | Me |
| 145 | F | —CH₂—CH₂— | Ph | Me |
| 146 | I | —CH₂—CH₂— | Ph | Me |
| 147 | prop-1-ynyl | —CH₂—CH₂— | Ph | Me |
| 148 | MeO | —CH₂—CH₂— | Ph | Me |
| 149 | EtO | —CH₂—CH₂— | Ph | Me |
| 150 | cyclopropyl | —CH₂—CH₂— | Ph | Me |
| 151 | Cl | —CH═CH— | Ph | Me |
| 152 | Br | —CH═CH— | Ph | Me |
| 153 | CN | —CH═CH— | Ph | Me |
| 154 | ethynyl | —CH═CH— | Ph | Me |
| 155 | F | —CH═CH— | Ph | Me |
| 156 | I | —CH═CH— | Ph | Me |
| 157 | prop-1-ynyl | —CH═CH— | Ph | Me |
| 158 | MeO | —CH═CH— | Ph | Me |
| 159 | EtO | —CH═CH— | Ph | Me |
| 160 | cyclopropyl | —CH═CH— | Ph | Me |

### Table 105

Table 105 provides 160 compounds of (IB) wherein R² is (1,1-dioxothietan-3-yl)methyl, and R¹, A, R⁵, R⁶ are as defined in Table P2 (above).

### Table 106

Table 106 provides 160 compounds of (IB) wherein R² is (2-oxo-1,3-dioxolan-4-yl)methyl, and R¹, A, R⁵, R⁶ are as defined in Table P2 (above).

### Table 107

Table 107 provides 160 compounds of (IB) wherein R² is (2-oxotetrahydrofuran-3-yl)methyl, and R¹, A, R⁵, R⁶ are as defined in Table P2 (above).

### Table 108

Table 108 provides 160 compounds of (IB) wherein R² is (5-oxotetrahydrofuran-2-yl)methyl, and R¹, A, R⁵, R⁶ are as defined in Table P2 (above).

### Table 109

Table 109 provides 160 compounds of (IB) wherein R² is (CH₂)₂S(O)2Me, and R¹, A, R⁵, R⁶ are as defined in Table P2 (above).

### Table 110

Table 110 provides 160 compounds of (IB) wherein R² is (CH₂)₂S(O)₂NHMe, and R¹, A, R⁵, R⁶ are as defined in Table P2 (above).

### Table 111

Table 111 provides 160 compounds of (IB) wherein R² is (CH₂)₂S(O)Me, and R¹, A, R⁵, R⁶ are as defined in Table P2 (above).

### Table 112

Table 112 provides 160 compounds of (IB) wherein R² is (E)-1-methylbut-2-enyl, and R¹, A, R⁵, R⁶ are as defined in Table P2 (above).

### Table 113

Table 113 provides 160 compounds of (IB) wherein R² is (S)—CHMeC(O)OMe, and R¹, A, R⁵, R⁶ are as defined in Table P2 (above).

### Table 114

Table 114 provides 160 compounds of (IB) wherein R² is 1-methylbut-2-enyl, and R¹, A, R⁵, R⁶ are as defined in Table P2 (above).

### Table 115

Table 115 provides 160 compounds of (IB) wherein R² is (Z)-2,3-dichloroallyl, and R¹, A, R⁵, R⁶ are as defined in Table P2 (above).

### Table 116

Table 116 provides 160 compounds of (IB) wherein R² is (Z)-3-chlorobut-2-enyl, and R¹, A, R⁵, R⁶ are as defined in Table P2 (above).

### Table 117

Table 117 provides 160 compounds of (IB) wherein R² is (R)—CHMeC(O)OMe, and R¹, A, R⁵, R⁶ are as defined in Table P2 (above).

### Table 118

Table 118 provides 160 compounds of (IB) wherein R² is 1,1-dimethylallyl, and R¹, A, R⁵, R⁶ are as defined in Table P2 (above).

### Table 119

Table 119 provides 160 compounds of (IB) wherein R² is 1,1-dimethylprop-2-ynyl, and R¹, A, R⁵, R⁶ are as defined in Table P2 (above).

### Table 120

Table 120 provides 160 compounds of (IB) wherein R² is 1,1-dioxothietan-3-yl, and R¹, A, R⁵, R⁶ are as defined in Table P2 (above).

### Table 121

Table 121 provides 160 compounds of (IB) wherein R² is 1,3-dioxolan-2-ylmethyl, and R¹, A, R⁵, R⁶ are as defined in Table P2 (above).

### Table 122

Table 122 provides 160 compounds of (IB) wherein R² is 1,3-dithian-5-yl, and R¹, A, R⁵, R⁶ are as defined in Table P2 (above).

### Table 123

Table 123 provides 160 compounds of (IB) wherein R² is 1-cyano-1-methyl-ethyl, and R¹, A, R⁵, R⁶ are as defined in Table P2 (above).

### Table 124

Table 124 provides 160 compounds of (IB) wherein R² is 1-cyano-2-methyl-propyl, and R¹, A, R⁵, R⁶ are as defined in Table P2 (above).

### Table 125

Table 125 provides 160 compounds of (IB) wherein R² is 1-methoxycarbonylpropyl, and R¹, A, R⁵, R⁶ are as defined in Table P2 (above).

### Table 126

Table 126 provides 160 compounds of (IB) wherein R² is 1-methyl-2-methylsulfanyl-ethyl, and R¹, A, R⁵, R⁶ are as defined in Table P2 (above).

### Table 127

Table 127 provides 160 compounds of (IB) wherein R² is 1-methyl-2-oxo-2-propoxy-ethyl, and R¹, A, R⁵, R⁶ are as defined in Table P2 (above).

### Table 128

Table 128 provides 160 compounds of (IB) wherein R² is 1-methyl-2-oxo-propyl, and R¹, A, R⁵, R⁶ are as defined in Table P2 (above).

### Table 129

Table 129 provides 160 compounds of (IB) wherein R² is 1-methylallyl, and R¹, A, R⁵, R⁶ are as defined in Table P2 (above).

### Table 130

Table 130 provides 160 compounds of (IB) wherein R² is 1-methylprop-2-ynyl, and R¹, A, R⁵, R⁶ are as defined in Table P2 (above).

### Table 131

Table 131 provides 160 compounds of (IB) wherein R² is 1-oxothietan-3-yl, and R¹, A, R⁵, R⁶ are as defined in Table P2 (above).

### Table 132

Table 132 provides 160 compounds of (IB) wherein R² is 2-(2,2-difluoroethylamino)-2-oxo-ethyl, and R¹, A, R⁵, R⁶ are as defined in Table P2 (above).

### Table 133

Table 133 provides 160 compounds of (IB) wherein R² is CHMeC(O)OMe, and R¹, A, R⁵, R⁶ are as defined in Table P2 (above).

### Table 134

Table 134 provides 160 compounds of (IB) wherein R² is 2-(methylsulfonimidoyl)ethyl, and R¹, A, R⁵, R⁶ are as defined in Table P2 (above).

### Table 135

Table 135 provides 160 compounds of (IB) wherein R² is 2,2,2-trifluoro-1-methyl-ethyl, and R¹, A, R⁵, R⁶ are as defined in Table P2 (above).

### Table 136

Table 136 provides 160 compounds of (IB) wherein R² is 2,2,2-trifluoroethyl, and R¹, A, R⁵, R⁶ are as defined in Table P2 (above).

### Table 137

Table 137 provides 160 compounds of (IB) wherein R² is 2,2,3,3,3-pentafluoropropyl, and R¹, A, R⁵, R⁶ are as defined in Table P2 (above).

### Table 138

Table 138 provides 160 compounds of (IB) wherein R² is 2,2,3,3-tetrafluoropropyl, and R¹, A, R⁵, R⁶ are as defined in Table P2 (above).

### Table 139

Table 139 provides 160 compounds of (IB) wherein R² is 2,2-difluorobutyl, and R¹, A, R⁵, R⁶ are as defined in Table P2 (above).

### Table 140

Table 140 provides 160 compounds of (IB) wherein R² is 2,2-difluoroethyl, and R¹, A, R⁵, R⁶ are as defined in Table P2 (above).

### Table 141

Table 141 provides 160 compounds of (IB) wherein R² is 2,2-difluoropropyl, and R¹, A, R⁵, R⁶ are as defined in Table P2 (above).

### Table 142

Table 142 provides 160 compounds of (IB) wherein R² is 2,2-dimethylbut-3-ynyl, and R¹, A, R⁵, R⁶ are as defined in Table P2 (above).

### Table 143

Table 143 provides 160 compounds of (IB) wherein R² is 2-allyloxy-1-methyl-2-oxo-ethyl, and R¹, A, R⁵, R⁶ are as defined in Table P2 (above).

### Table 144

Table 144 provides 160 compounds of (IB) wherein R² is 2-carboxy-3,3,3-trifluoro-propyl, and R¹, A, R⁵, R⁶ are as defined in Table P2 (above).

### Table 145

Table 145 provides 160 compounds of (IB) wherein R² is 2-cyanoallyl, and R¹, A, R⁵, R⁶ are as defined in Table P2 (above).

### Table 146

Table 146 provides 160 compounds of (IB) wherein R² is 2-cyanoethyl, and R¹, A, R⁵, R⁶ are as defined in Table P2 (above).

### Table 147

Table 147 provides 160 compounds of (IB) wherein R² is 2-ethoxy-1-methyl-2-oxo-ethyl, and R¹, A, R⁵, R⁶ are as defined in Table P2 (above).

### Table 148

Table 148 provides 160 compounds of (IB) wherein R² is 2-ethoxy-2-oxo-ethyl, and R¹, A, R⁵, R⁶ are as defined in Table P2 (above).

### Table 149

Table 149 provides 160 compounds of (IB) wherein R² is 2-fluoroallyl, and R¹, A, R⁵, R⁶ are as defined in Table P2 (above).

### Table 150

Table 150 provides 160 compounds of (IB) wherein R² is 2-fluoroethyl, and R¹, A, R⁵, R⁶ are as defined in Table P2 (above).

### Table 151

Table 151 provides 160 compounds of (IB) wherein R² is 2-methoxy-1-methyl-ethyl, and R¹, A, R⁵, R⁶ are as defined in Table P2 (above).

### Table 152

Table 152 provides 160 compounds of (IB) wherein R² is 2-methoxyethyl, and R¹, A, R⁵, R⁶ are as defined in Table P2 (above).

### Table 153

Table 153 provides 160 compounds of (IB) wherein R² is 2-methylallyl, and R¹, A, R⁵, R⁶ are as defined in Table P2 (above).

### Table 154

Table 154 provides 160 compounds of (IB) wherein R² is 2-methylsulfanylethyl, and R¹, A, R⁵, R⁶ are as defined in Table P2 (above).

### Table 155

Table 155 provides 160 compounds of (IB) wherein R² is 2-oxobutyl, and R¹, A, R⁵, R⁶ are as defined in Table P2 (above).

### Table 156

Table 156 provides 160 compounds of (IB) wherein R² is 3,3,3-trifluoropropyl, and R¹, A, R⁵, R⁶ are as defined in Table P2 (above).

### Table 157

Table 157 provides 160 compounds of (IB) wherein R² is 3-ethoxy-3-oxo-propyl, and R¹, A, R⁵, R⁶ are as defined in Table P2 (above).

### Table 158

Table 158 provides 160 compounds of (IB) wherein R² is 3-fluoropropyl, and R¹, A, R⁵, R⁶ are as defined in Table P2 (above).

### Table 159

Table 159 provides 160 compounds of (IB) wherein R² is 3-methoxy-2-methyl-3-oxo-propanoyl, and R¹, A, R⁵, R⁶ are as defined in Table P2 (above).

### Table 160

Table 160 provides 160 compounds of (IB) wherein R² is 3-methylbut-2-enyl, and R¹, A, R⁵, R⁶ are as defined in Table P2 (above).

### Table 161

Table 161 provides 160 compounds of (IB) wherein R² is 3-oxocyclohexen-1-yl, and R¹, A, R⁵, R⁶ are as defined in Table P2 (above).

### Table 162

Table 162 provides 160 compounds of (IB) wherein R² is 3-oxocyclopenten-1-yl, and R¹, A, R⁵, R⁶ are as defined in Table P2 (above).

### Table 163

Table 163 provides 160 compounds of (IB) wherein R² is 3-t-butoxy-3-oxo-propyl, and R¹, A, R⁵, R⁶ are as defined in Table P2 (above).

### Table 164

Table 164 provides 160 compounds of (IB) wherein R² is 2-chloroallyl, and R¹, A, R⁵, R⁶ are as defined in Table P2 (above).

### Table 165

Table 165 provides 160 compounds of (IB) wherein R² is 4,4,4-trifluorobutyl, and R¹, A, R⁵, R⁶ are as defined in Table P2 (above).

### Table 166

Table 166 provides 160 compounds of (IB) wherein R² is 4-methoxybut-2-ynyl, and R¹, A, R⁵, R⁶ are as defined in Table P2 (above).

### Table 167

Table 167 provides 160 compounds of (IB) wherein R² is (5-methyl-2-oxo-1,3-dioxol-4-yl)methyl, and R¹, A, R⁵, R⁶ are as defined in Table P2 (above).

### Table 168

Table 168 provides 160 compounds of (IB) wherein R² is acetyl, and R¹, A, R⁵, R⁶ are as defined in Table P2 (above).

### Table 169

Table 169 provides 160 compounds of (IB) wherein R² is allyl, and R¹, A, R⁵, R⁶ are as defined in Table P2 (above).

### Table 170

Table 170 provides 160 compounds of (IB) wherein R² is benzyl, and R¹, A, R⁵, R⁶ are as defined in Table P2 (above).

### Table 171

Table 171 provides 160 compounds of (IB) wherein R² is but-2-ynyl, and R¹, A, R⁵, R⁶ are as defined in Table P2 (above).

### Table 172

Table 172 provides 160 compounds of (IB) wherein R² is but-3-ynyl, and R¹, A, R⁵, R⁶ are as defined in Table P2 (above).

### Table 173

Table 173 provides 160 compounds of (IB) wherein R² is C(O)OMe, and R¹, A, R⁵, R⁶ are as defined in Table P2 (above).

### Table 174

Table 174 provides 160 compounds of (IB) wherein R² is C(O)Ot-Bu, and R¹, A, R⁵, R⁶ are as defined in Table P2 (above).

### Table 175

Table 175 provides 160 compounds of (IB) wherein R² is CH(CO₂Et)₂, and R¹, A, R⁵, R⁶ are as defined in Table P2 (above).

### Table 176

Table 176 provides 160 compounds of (IB) wherein R² is CH(S), and R¹, A, R⁵, R⁶ are as defined in Table P2 (above).

### Table 177

Table 177 provides 160 compounds of (IB) wherein R² is CH₂C(O)Me, and R¹, A, R⁵, R⁶ are as defined in Table P2 (above).

### Table 178

Table 178 provides 160 compounds of (IB) wherein R² is CH₂C(O)NHMe, and R¹, A, R⁵, R⁶ are as defined in Table P2 (above).

### Table 179

Table 179 provides 160 compounds of (IB) wherein R² is CH₂C(O)OH, and R¹, A, R⁵, R⁶ are as defined in Table P2 (above).

### Table 180

Table 180 provides 160 compounds of (IB) wherein R² is CH₂C(O)OMe, and R¹, A, R⁵, R⁶ are as defined in Table P2 (above).

### Table 181

Table 181 provides 160 compounds of (IB) wherein R² is CH₂CH₂OEt, and R¹, A, R⁵, R⁶ are as defined in Table P2 (above).

### Table 182

Table 182 provides 160 compounds of (IB) wherein R² is CH₂CN, and R¹, A, R⁵, R⁶ are as defined in Table P2 (above).

### Table 183

Table 183 provides 160 compounds of (IB) wherein R² is CH₂S(O)₂NHMe, and R¹, A, R⁵, R⁶ are as defined in Table P2 (above).

### Table 184

Table 184 provides 160 compounds of (IB) wherein R² is cyclobutyl, and R¹, A, R⁵, R⁶ are as defined in Table P2 (above).

### Table 185

Table 185 provides 160 compounds of (IB) wherein R² is cyclopropyl, and R¹, A, R⁵, R⁶ are as defined in Table P2 (above).

### Table 186

Table 186 provides 160 compounds of (IB) wherein R² is ethyl, and R¹, A, R⁵, R⁶ are as defined in Table P2 (above).

### Table 187

Table 187 provides 160 compounds of (IB) wherein R² is formyl, and R¹, A, R⁵, R⁶ are as defined in Table P2 (above).

### Table 188

Table 188 provides 160 compounds of (IB) wherein R² is hydrogen, and R¹, A, R⁵, R⁶ are as defined in Table P2 (above).

### Table 189

Table 189 provides 160 compounds of (IB) wherein R² is isobutyl, and R¹, A, R⁵, R⁶ are as defined in Table P2 (above).

### Table 190

Table 190 provides 160 compounds of (IB) wherein R² is isopropyl, and R¹, A, R⁵, R⁶ are as defined in Table P2 (above).

### Table 191

Table 191 provides 160 compounds of (IB) wherein R² is methyl, and R¹, A, R⁵, R⁶ are as defined in Table P2 (above).

### Table 192

Table 192 provides 160 compounds of (IB) wherein R² is n-Bu, and R¹, A, R⁵, R⁶ are as defined in Table P2 (above).

### Table 193

Table 193 provides 160 compounds of (IB) wherein R² is n-hexyl, and R¹, A, R⁵, R⁶ are as defined in Table P2 (above).

### Table 194

Table 194 provides 160 compounds of (IB) wherein R² is n-Pr, and R¹, A, R⁵, R⁶ are as defined in Table P2 (above).

### Table 195

Table 195 provides 160 compounds of (IB) wherein R² is oxetan-2-ylmethyl, and R¹, A, R⁵, R⁶ are as defined in Table P2 (above).

### Table 196

Table 196 provides 160 compounds of (IB) wherein R² is oxetan-3-yl, and R¹, A, R⁵, R⁶ are as defined in Table P2 (above).

### Table 197

Table 197 provides 160 compounds of (IB) wherein R² is oxetan-3-ylmethyl, and R¹, A, R⁵, R⁶ are as defined in Table P2 (above).

### Table 198

Table 198 provides 160 compounds of (IB) wherein R² is cyclobutylmethyl, and R¹, A, R⁵, R⁶ are as defined in Table P2 (above).

### Table 199

Table 199 provides 160 compounds of (IB) wherein R² is pent-2-ynyl, and R¹, A, R⁵, R⁶ are as defined in Table P2 (above).

### Table 200

Table 200 provides 160 compounds of (IB) wherein R² is pent-4-ynyl, and R¹, A, R⁵, R⁶ are as defined in Table P2 (above).

### Table 201

Table 201 provides 160 compounds of (IB) wherein R² is propargyl, and R¹, A, R⁵, R⁶ are as defined in Table P2 (above).

### Table 202

Table 202 provides 160 compounds of (IB) wherein R² is t-Bu, and R¹, A, R⁵, R⁶ are as defined in Table P2 (above).

### Table 203

Table 203 provides 160 compounds of (IB) wherein R² is tetrahydrofuran-3-ylmethyl, and R¹, A, R⁵, R⁶ are as defined in Table P2 (above).

### Table 204

Table 204 provides 160 compounds of (IB) wherein R² is tetrahydropyran-4-yl, and R¹, A, R⁵, R⁶ are as defined in Table P2 (above).

### Table 205

Table 205 provides 160 compounds of (IB) wherein R² is tetrahydrothiophen-2-ylmethyl, and R¹, A, R⁵, R⁶ are as defined in Table P2 (above).

### Table 206

Table 206 provides 160 compounds of (IB) wherein R² is tetrahydrothiophen-3-ylmethyl, and R¹, A, R⁵, R⁶ are as defined in Table P2 (above).

### Table 207

Table 207 provides 160 compounds of (IB) wherein R² is thietan-3-yl, and R¹, A, R⁵, R⁶ are as defined in Table P2 (above).

### Table 208

Table 208 provides 160 compounds of (IB) wherein R² is thietan-3-ylmethyl, and R¹, A, R⁵, R⁶ are as defined in Table P2 (above).

The compounds of the invention may be prepared by a variety of methods as shown in the following schemes.

Compounds of formula IA, wherein R¹, R², and A are defined as for formula I above and R³═R⁴═H, may be prepared starting from compounds of formula IIa, wherein A is defined as for formula I above, X stands for halogen, preferably Br, Cl or I (Scheme 1). PG in formula IIa is stands for a protecting group, preferably a tert-butoxycarbonyl, ethoxycarbonyl or benzyloxycarbonyl group (see e.g. T. W. Greene et al. “Protective Groups in Organic Synthesis”, 3^{rd} edition 1999 by J. Wiley & Sons). Compounds of formula IIa may be subjected to a deprotection reaction (e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons) to furnish a compound of formula IIb, wherein A and X are defined as for compounds of formula IIa. Subsequent alkylation with an alkylating agent of general formula R²-LG, wherein LG stands for a leaving group such as CI, Br, I, OMs, OTs or, OTf, in the presence of an organic or inorganic base, preferably K₂CO₃ or iPr₂NEt, may provide compounds of general IIc, wherein X stands for halogen and A and R² are defined as for formula I above. Compounds of formula IIc may be converted to compounds of formula II, wherein R¹, R² and A are defined as for formula I above (see Scheme 9 for a detailed description of this process). Compounds of formula IA, wherein R³═R⁴═H and R¹, R², and A are defined as for formula I above, may be prepared by reacting compounds of formula II with a thiolating agent using methods described in the literature: (NH₄)₂Sₓ, pyridine (H. Foks et al., Heterocycles 2009, 78, 961); HS—P(S)(OEt)₂, dioxane (L. D. S. Yadav et al., Tetrahedron Lett. 2012, 53, 7113); J. Pesti et al., Org. Proc. Res. Dev. 2009, 13, 716); H₂S, aq. NH₃, EtOH (K. P. Sasmal et al., Bioorg. Med. Chem. Lett. 2011, 21, 4913; H. Z. Boeini et al., Synlett 2010, 2861); Lawesson's Reagent, BF₃OEt₂, DME, THF (W. Schmide et al., Synthesis 2008, 4012). Alternatively, compounds of formula IIc may be converted into compounds of formula IAa, wherein X stands for halogen, R³═R⁴═H, R² and A are defined as for formula I above, by reaction with thiolating agents using methods that have been described above for the conversion of compound of formula II into compounds of formula IA.

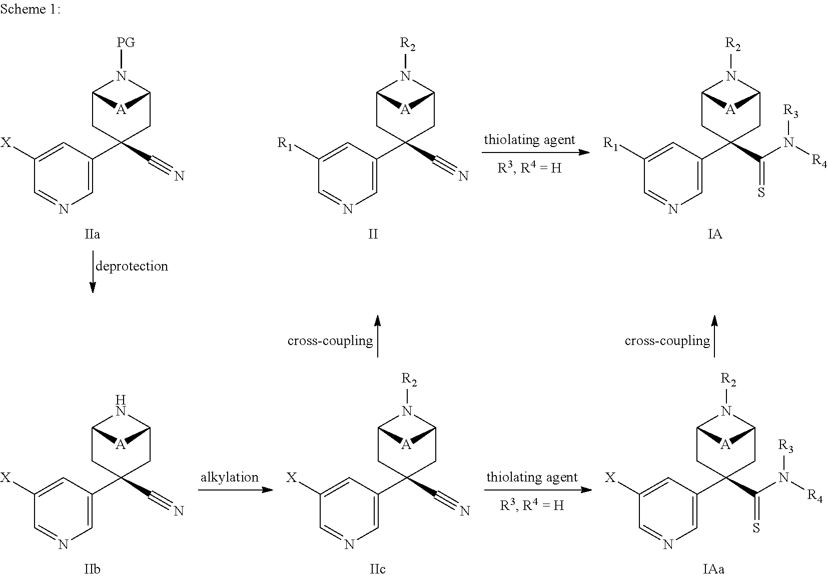

Compounds of formula IAa, wherein X stand for halogen and preferably for Br, I, may be further converted into compounds of formula IA, wherein R³═R⁴═H and R¹, R², and A are defined as for formula I above, by using cross-coupling reactions, i.e. Pd-catalyzed cyanation or Pd-catalyzed Sonogashira reactions; typical conditions of these conversions can be found in Scheme 9 for the conversion of compounds of formula IIc into compounds of formulas IIf, IIg, and IIh.

Alternatively, compounds of formula IAa may be prepared by methods shown in Scheme 2. Compounds of formula IAb, wherein R3=R4=H, X stands for halogen, PG is a protecting group preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons) and A is defined as for formula I, may be prepared by reaction of compounds of formula IIa with a thiolating agent using methods that have been described above for the conversion of compound of formula II into compounds of formula IA (see Scheme 1). Compounds of formula IAb may be subjected to a deprotection reaction (e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons) to furnish a compound of formula IAC, wherein R3=R4=H, X stands for halogen, and A is defined as for formula I.

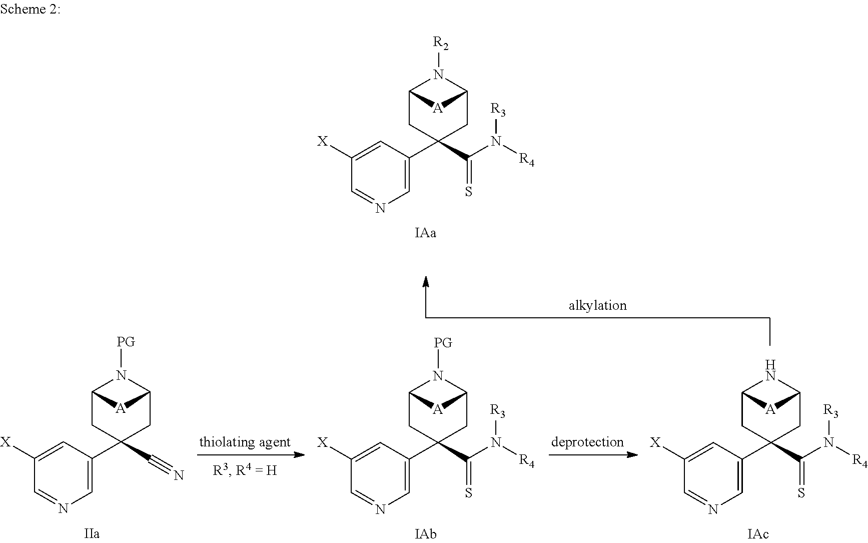

Subsequent alkylation of compounds of formula IAc with an alkylating agent of general formula R²-LG, wherein LG stands for a leaving group such as CI, Br, I, OMs, OTs or, OTf, in the presence of an organic or inorganic base, preferably K₂CO₃ or iPr₂NEt, may provide compounds of formula IAa.

In another alternative, compounds of formula IA, wherein R¹, R², and A are defined as for formula I above and R³═R⁴═H, may be prepared according to methods described in Scheme 3. Compounds of formula IIa, wherein X stands for halogen, PG is a protecting group, preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons), and A is defined as for formula I, may be converted into compounds of formula IAb, wherein R³═R⁴═H, X stands for halogen, PG is a protecting group preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons) and A is defined as for formula I, with a thiolating agent using methods that have been described above for the conversion of compound of formula II into compounds of formula IA (see Scheme 1). Compounds of formula IAb may then be subjected to cross-coupling reactions, i.e. Pd-catalyzed cyanation or Pd-catalyzed Sonogashira reactions, to yield compounds of formula IAd, wherein R³═R⁴═H, R¹ and A are defined as in formula I and PG is a protecting group preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons). Typical conditions of these cross-coupling reactions can be found in Scheme 9 for the conversion of compounds of formula IIc into compounds of formulas IIf, IIg, and IIh. Alternatively, compounds of formula IAd may be obtained from compound IIa by reversing the order of steps described above using otherwise identical conditions: Following this approach, IIa may be converted into compounds of formula IId, wherein R¹ and A are defined as for formula I and PG is a protecting group as defined for formula IIa, via a transition-metal catalyzed cross-coupling reaction. Then, conversion of compounds of formula IId may be converted into compounds of formula IAd in the presence of a thiolating reagent using methods described above. Compounds of formula IAd may be further converted into compounds of formula IAe, wherein R³═R⁴═H, A and R¹ are defined as for formula I by a deprotection reaction (e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons). Subsequent alkylation of compounds of formula IAe with an alkylating agent of general formula R²-LG, wherein LG stands for a leaving group such as CI, Br, I, OMs, OTs or, OTf, in the presence of an organic or inorganic base, preferably K₂CO₃ or iPr₂NEt, may provide compounds of formula IA, wherein R³═R⁴═H, A, R² and R¹ are defined as for formula I.

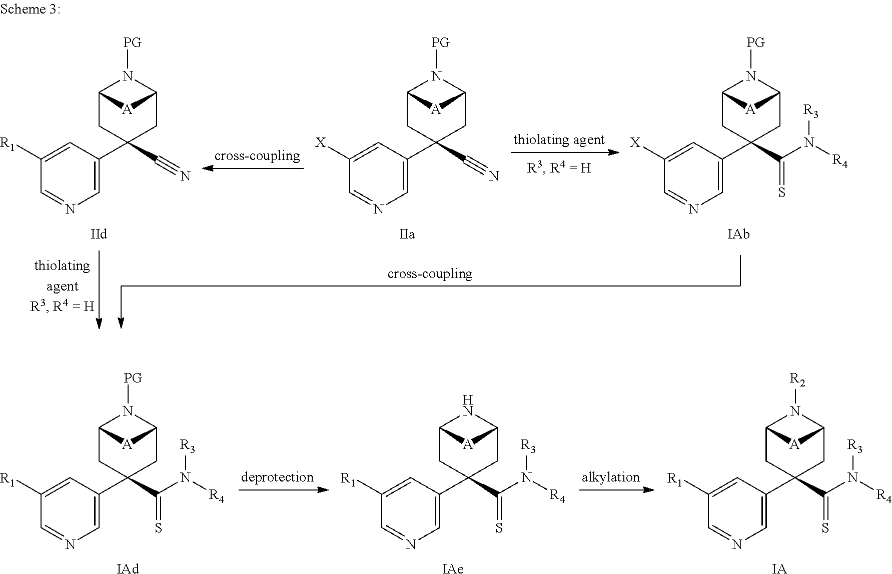

In yet an alternative procedure, compounds of formula IAe, wherein R³═R⁴═H, A and R¹ are defined as for formula I, may be obtained from compounds of formula IIe, wherein R¹ and A are defined as in formula I, as described in Scheme 4. Compounds of formula IIe may be obtained from compounds of formula IId, wherein R¹, A and PG are defined as above, by a deprotection reaction (e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons). Alternatively, compounds of formula IIe may be obtained starting from compounds of formula IIb, wherein X and A are defined as above, by using cross-coupling reactions, i.e. Pd-catalyzed cyanation or Pd-catalyzed Sonogashira reactions; typical conditions of these conversions can be found in Scheme 9 for the conversion of compounds of formula IIc into compounds of formulas IIf, IIg, and IIh. Compounds of formula IAe may then be obtained by reaction of compounds of formula IIe with a thiolating agent using methods that have been described above for the conversion of compound of formula II into compounds of formula IA (see Scheme 1). Alternatively, compounds of formula IAe may be obtained may be obtained from compound IId by reversing the order of steps described above using otherwise identical conditions: Following this approach, reaction of compounds of formula IIb with a thiolating reagent may furnish compounds of formula IAc, wherein R³═R⁴═H, X stands for halogen and A is defined as in formula I. Subsequent cross-coupling reaction as described above may then deliver compounds of formula IAe.

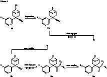

In yet another alternative procedure, compounds of formula IA, wherein R¹, R², R³, R⁴ and A are defined as for compounds of formula I, may be obtained starting from compounds of formula IIe (Scheme 5). Compounds of formula IIe, wherein A and R¹ are defined as for compounds of formula I may be subjected to an alkylation reaction with an alkylating agent of general formula R²-LG, wherein LG stands for a leaving group such as CI, Br, I, OMs, OTs or, OTf, in the presence of an organic or inorganic base, preferably K₂CO₃ or iPr₂NEt, to provide compounds of formula II, wherein R¹, R² and A are defined as for formula I. Compounds of formula II may then be treated with a thiolating agent using methods that have been described above for the conversion of compound of formula II into compounds of formula IA to provide compounds of general formula IAf, wherein R¹, R² and A are defined as for formula I. Compounds of formula IA may be prepared by amine exchange of unsubstituted thioamides IAf with amines of formula R³R⁴NH, wherein R³ and R⁴ are defined in formula I, according to procedures reported in the literature: M. H. Klingele et al., Eur. J. Org. Chem. 2004, 3422; J. Spychala et al., Tetrahedron 2000, 56, 7981; A. C. W. Curran et al., J. Chem. Soc., Perkin Trans. I, 1976, 977.

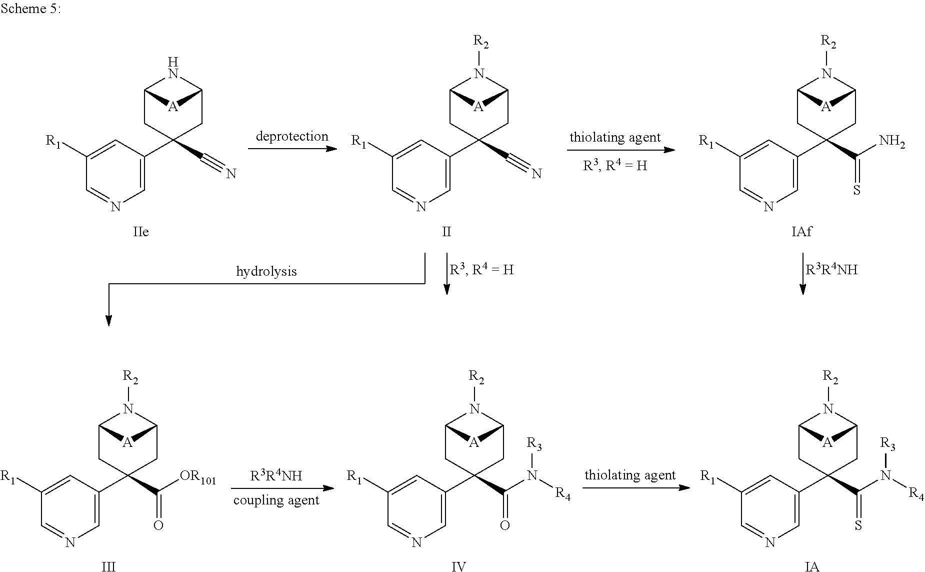

In yet another process, compounds of formula II may be subjected to partial hydrolysis under basic conditions, using an inorganic base, preferably KOH or NaOH, in the presence or absence of a catalytic additive such as aqueous hydrogen peroxide, in a polar solvent, preferably EtOH or water or mixtures thereof at temperatures between 0° C. and 120° C., preferably between 80 and 100° C. to give the primary amide of formula IV, wherein R³═R⁴═H, R¹, R² and A are defined as in formula I. Substituted amides of formula IV, wherein R¹, R², R³, R⁴, and A are defined as in formula I may be prepared by complete hydrolysis of compounds of formula II to give compounds of formula III, wherein R¹, R² and A are defined as in formula I and R¹⁰¹ represents a hydrogen or a C₁-C₄alkyl group, preferably hydrogen. In order to obtain carboxylic acid derivatives of formula III, compounds of formula II may be hydrolyzed under basic conditions as described above followed by treatment with acid, preferably an aqueous mineral acid such as aqueous HCl or aqueous sulfuric acid. Alternative procedures have been described in WO 98/25923. Compounds of general formula III may be converted into compounds of formula IV, wherein R¹, R², R³, R⁴, and A are defined as in formula I according to known methods, e.g. by using peptide coupling procedures if R¹⁰¹═H: For example by reaction with and amine R³R⁴NH, wherein R³ and R⁴ are defined as in formula I, in the presence of a coupling reagent, such as DCC (N,N′-dicyclohexylcarbodiimide), EDC (1-ethyl-3-[3-dimethylamino-propyl]carbodiimide hydrochloride) or BOP—Cl (bis(2-oxo-3-oxazolidinyl)phosphonic chloride), in the presence of a base, such as pyridine, triethylamine, 4-(dimethylamino)pyridine or diisopropylethylamine, and optionally in the presence of a nucleophilic catalyst, such as hydroxybenzotriazole. The reaction is advantageously carried out in an organic solvent such as tetrahydrofuran or N,N-dimethylformamide in a temperature range from approximately −80° C. to approximately +80° C., preferably from approximately −20° C. to approximately +40° C., in many cases in the range between 0° C. and ambient temperature. Compounds of general formula IV may then be converted into compounds of formula IA, wherein R¹, R², R³, R⁴, and A are defined as in formula I by applying methods described in the literature: See for example WO 2008/71646 (p. 80), A. B. Charette et al., J. Org. Chem. 2003, 68, 5792; B. Kaboudin et al., Synlett 2011, 2807; using Lawesson's reagent, T. Ozturk et al., Chem. Rev. 2007, 107, 5210.
In a similar fashion compounds of formula IA may be prepared from compounds of formula IAd, wherein R¹, R³, R⁴ and A are defined as in formula I and PG stands for a protecting group, preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons) or from compounds of formula IV, wherein R¹, R², R³, R⁴, and A are defined as in formula I Both of these compounds may be available in from compounds of formula IId, wherein R¹ and A are defined as in formula I and PG stands for a protecting group as defined above (Scheme 6). Conversion of compounds of formula IV, and, respectively, formula IAd into compounds of formula IA has been described in previous schemes above. Compounds of formula IId may be converted in two steps into compounds of formula IVa, wherein R¹, R³, R⁴, and A are defined as for formula I and PG stands for a protecting group preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons). In the first step, hydrolysis under aqueous conditions such as described in Scheme 5 may lead to compounds of formula IIIa, wherein R¹ and A are defined as for formula I, PG stands for a protecting group, and R¹⁰¹ represents a hydrogen or a C₁-C₄alkyl group, preferably hydrogen. Alternative procedures have been described in WO 98/25923. Compounds of general formula IIIa may be converted into compounds of formula IV, wherein R¹, R³, R⁴, and A are defined as in formula I and PG stands for a protecting group as defined above, according to known methods, e.g. by using peptide coupling procedures if R¹⁰¹═H: For example by reaction with and amine R³R⁴NH, wherein R³ and R⁴ are defined as in formula I, in the presence of a coupling reagent, such as DCC (N,N′-dicyclohexylcarbodiimide), EDC (1-ethyl-3-[3-dimethylamino-propyl]carbodiimide hydrochloride) or BOP—Cl (bis(2-oxo-3-oxazolidinyl)phosphonic chloride), in the presence of a base, such as pyridine, triethylamine, 4-(dimethylamino)pyridine or diisopropylethylamine, and optionally in the presence of a nucleophilic catalyst, such as hydroxybenzotriazole. The reaction is advantageously carried out in an organic solvent such as tetrahydrofuran or N,N-dimethylformamide in a temperature range from approximately −80° C. to approximately +80° C., preferably from approximately −20° C. to approximately +40° C., in many cases in the range between 0° C. and ambient temperature. Compounds of formula IVa may then be reacted with a thiolating reagent using methods described in the literature (see for example WO 2008/71646 (p. 80), A. B. Charette et al., J. Org. Chem. 2003, 68, 5792; B. Kaboudin et al., Synlett 2011, 2807; using Lawesson's reagent, T. Ozturk et al., Chem. Rev. 2007, 107, 5210) to obtain compounds of formula IAd. Alternatively, compounds of formula IVa may be subjected to a deprotection reaction (e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons) to give compound of formula IVb, wherein R¹, R³, R⁴, and A are defined as for formula I. Alkylation of compounds of formula IVb with an alkylating agent of general formula R²-LG, wherein LG stands for a leaving group such as CI, Br, I, OMs, OTs or, OTf, in the presence of an organic or inorganic base, preferably K₂CO₃ or iPr₂NEt, may then provide compounds of formula IV.

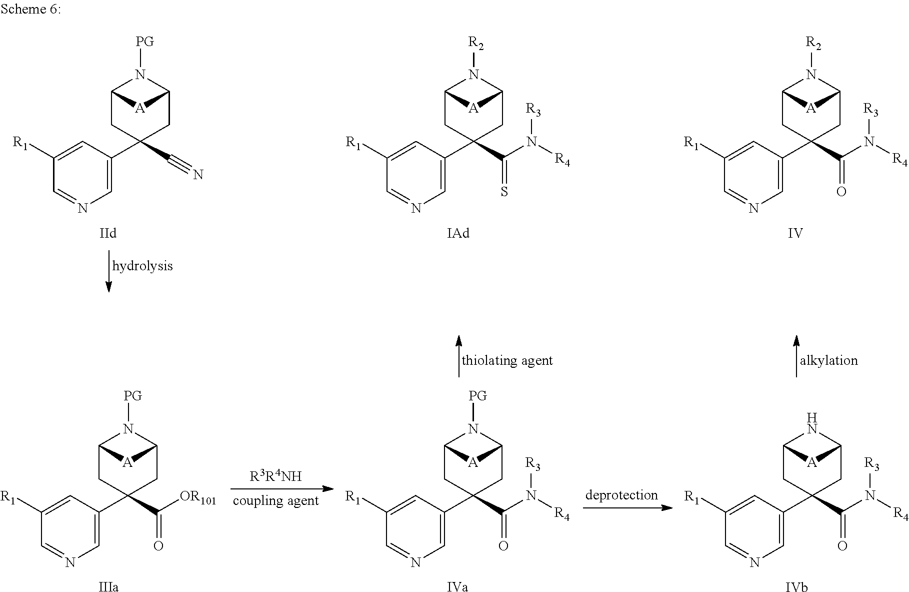

Compounds of general formula IB, wherein R¹, R², R⁵, R⁶, and A are defined as above may be prepared from compound of formula IBa, wherein R¹, R², R⁵ and A are defined as for formula IB, by reaction with an alkylating reagent of general formula R⁶-LG, wherein R⁶ is defined as in formula IB and LG stands for a leaving group such as CI, Br, I, OMs, OTs or, OTf, in the presence of an organic or inorganic base, preferably NaH, K₂CO₃ or iPr₂NEt. Compounds of formula IBa can be prepared analogously as described for compounds of formula IA, wherein R³═H.

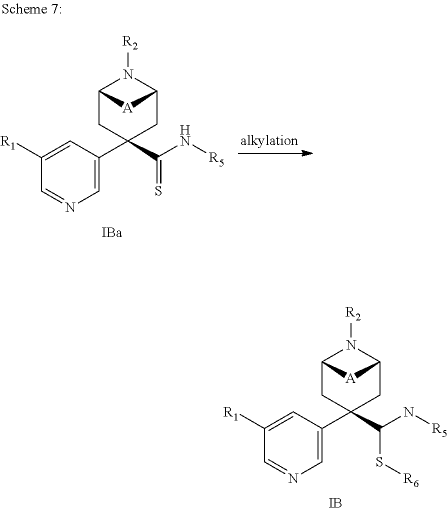

The compounds of general formula II may be prepared according to procedures described in WO9637494, WO9825924, WO02057262 and GB2372744 or by a variety of methods as shown in the following schemes.

Compounds of general formula II, wherein R¹, R² and A are defined as in formula I may be prepared according to Scheme 8 starting from compound V. The synthesis of compounds of formula V, wherein R² and A are defined as for formula I, have been described in WO9637494. Compounds of formula V may be converted to compounds of formula VI, wherein R² and A are defined as for formula I, according to known procedures described in WO9637494 and J. Org. Chem. 1977, 42, 3114. Compounds of formula VI may then react with compounds of formula VII, wherein R¹ is defined as in formula I, preferably halogen, and LG stands for a leaving group, preferably, halogen, most preferably F or CI, in the presence of a base such as NaNH₂, LDA or LiHMDS to give compounds of formula II.

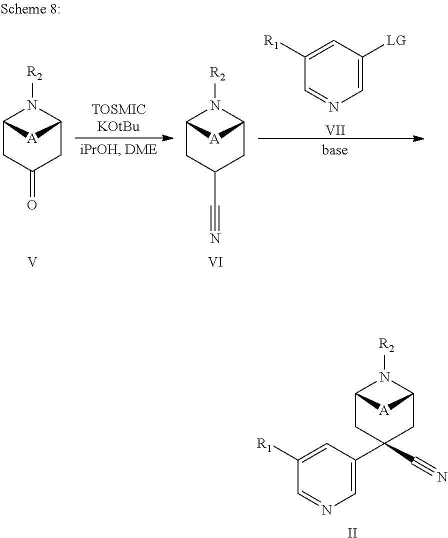

Alternatively, compounds of formula VI may be converted by methods described in Scheme 9. Compounds of formula VI may be reacted with compounds of formula Vila, wherein X is defined as halogen, preferably Br or I, and LG stands for a leaving group, preferably, halogen, most preferably F, in the presence of a base such as NaNH₂, LDA or LiHMDS to give compounds of formula IIc, wherein X is halogen, preferably Br or I, and A and R² are defined as in in formula I. Compounds of formula IIc may be subjected to a cyanation reaction. This cyanation reaction, preferably transition-metal catalyzed, and most preferably palladium-catalyzed, using Pd-precursors such as Pd₂(dba)₃, Pd(OAc)₂, Pd(PPh₃)₄, Pd(PPh₃)₂Cl₂, and a ligand, preferably a phosphine, a cyanide source such as Zn(CN)₂, K₄[Fe(CN)₆], and additives such as zinc in a polar solvent such as DMF, DMA, or NMP (see for example: WO03059269 or WO07139230) then may furnish compounds of formula IIf, wherein R² and A are defined as in formula I.

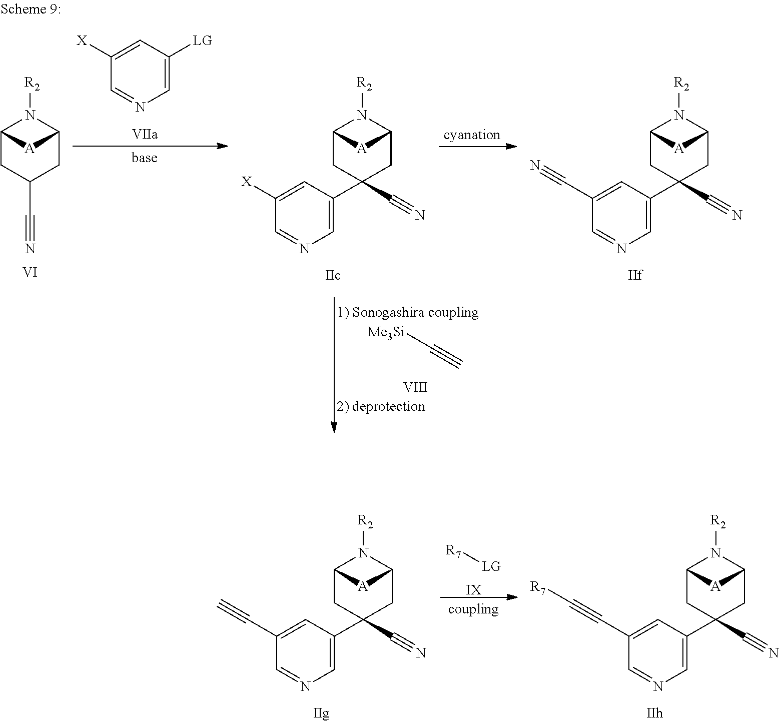

Alternatively, compounds of formula IIc may be subjected to a alkynylation reaction, preferably transition-metal catalyzed, and most preferably palladium-catalyzed, using Pd-precursors such as Pd₂(dba)₃, Pd(OAc)₂, Pd(PPh₃)₄, Pd(PPh₃)₂Cl₂, and a ligand, preferably a phosphine, in the presence of a base and a compound of formula VIII, followed by treatment of the resulting product with a base, e.g., KOH or K₂CO₃, to give a compound of general formula IIg, wherein R² and A are defined as in formula I. Methods for this Sonogashira reaction have been reported in the literature, see for example Chem. Rev. 2007, 107, 874; Org. Lett. 2004, 6, 889 and Bioorg. Med. Chem. 2004, 13, 197. Subsequent substitution of compounds of formula IIg with compounds of formula IX, wherein R⁷ is defined as for formula I and LG is a leaving group, preferably a halogen, most preferably I, may be conducted following procedures reported in the literature (C. Meyer et al., Org. Lett. 2011, 13, 956) to give compounds of formula IIh, wherein R², R⁷ and A are defined as in formula I.
In a related approach, compound of general formula IIf may be prepared starting from compounds of general formula VIa, wherein A is defined as in formula I and PG stands for a protecting group, preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons). Compounds of formula Via may be reacted with compounds of formula Vila, wherein X is defined as halogen, preferably Br or I, and LG stands for a leaving group, preferably, halogen, most preferably F, in the presence of a base such as NaNH₂, LDA or LiHMDS to give compounds of formula IIa, wherein X is halogen, preferably Br or I, and Pg stand for a protecting group as defined above. Compounds of formula IIa may be subjected to a cyanation reaction. Said cyanation reaction, preferably transition-metal catalyzed, and most preferably palladium-catalyzed, using Pd-precursors such as Pd₂(dba)₃, Pd(OAc)₂, Pd(PPh₃)₄, Pd(PPh₃)₂Cl₂, and a ligand, preferably a phosphine, a cyanide source such as Zn(CN)₂, K₄[Fe(CN)₆], and additives such as zinc in a polar solvent such as DMF, DMA, or NMP (see for example: WO03059269 or WO07139230) then may furnish compounds of formula IIi, wherein A is defined as in formula I and PG stands for a protecting group as defined above. Compounds of formula IIi may then be subjected to a deprotection reaction, e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons), leading to compounds of formula IIj, wherein A is defined as in formula I. Compounds of formula IIf, wherein R² and A are defined as in formula I may be obtained by reaction with an alkylating reagent of general formula R²-LG, wherein R² is defined as in formula I and LG stands for a leaving group such as CI, Br, I, OMs, OTs or, OTf, in the presence of an organic or inorganic base, preferably K₂CO₃ or iPr₂NEt.

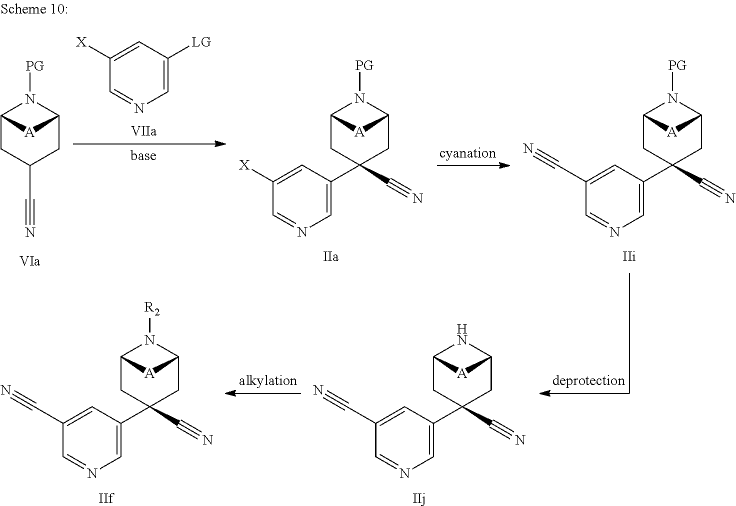

In yet another alternative, compounds of general formula IIg and IIh, wherein R², R⁷ and A are defined as for formula I, may be prepared following the process described in Scheme 11. Starting from compound IIa, by means of an alkynylation reaction, preferably transition-metal catalyzed, and most preferably palladium-catalyzed, using Pd-precursors such as Pd₂(dba)₃, Pd(OAc)₂, Pd(PPh₃)₄, Pd(PPh₃)₂Cl₂, and a ligand, preferably a phosphine, in the presence of a base and a compound of formula VIII, followed by treatment of the resulting product with a base, e.g., KOH or K₂CO₃, compounds of general formula IIm, wherein A is defined as in formula I and PG stands for a protecting group. Methods for this Sonogashira reaction have been reported in the literature, see for example Chem. Rev. 2007, 107, 874; Org. Lett. 2004, 6, 889 and Bioorg. Med. Chem. 2004, 13, 197. Compounds of formula IIn may be subjected to a deprotection reaction, e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons), leading to compounds of formula IIn, wherein A is defined as for formula I. Compounds of formula IIg may then be obtained by reaction with an alkylating reagent of general formula R²-LG, wherein R² is defined as in formula I and LG stands for a leaving group such as CI, Br, I, OMs, OTs or, OTf, in the presence of an organic or inorganic base, preferably K₂CO₃ or iPr₂NEt. Substituted acetylenes may be obtained by reaction of compounds of formula IIm with compounds of formula IX, wherein R⁷ is defined as for formula I and LG is a leaving group, preferably a halogen, most preferably I, following procedures reported in the literature (C. Meyer et al., Org. Lett. 2011, 13, 956) to give compounds of formula IIo, wherein R⁷ and A are defined as in formula I and PG stands for a protecting group. Compounds of formula Ho may be subjected to a deprotection reaction, e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons), leading to compounds of formula IIp, wherein R⁷ and A is defined as for formula I. Compounds of formula IIh may then be obtained by reaction with an alkylating reagent of general formula R²-LG, wherein R² is defined as in formula I and LG stands for a leaving group such as CI, Br, I, OMs, OTs or, OTf, in the presence of an organic or inorganic base, preferably K₂CO₃ or iPr₂NEt.

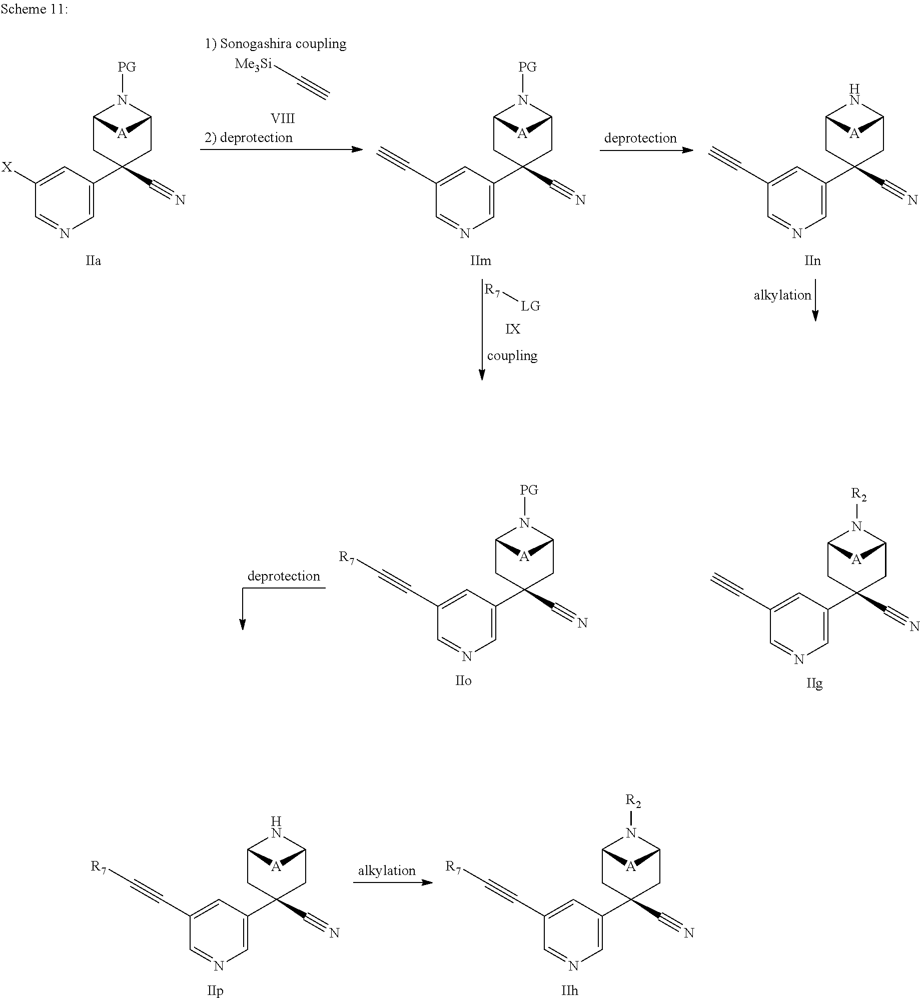

Compounds of formula VI and VIb may be prepared as shown in Scheme 12. Compounds of formula Vb, wherein PG stands for a protecting group, preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons) and A is defined as —CH₂—CH₂— are known compounds and may be prepared according to literature procedures, see, e.g., Tetrahedron 2002, 58, 5669 or US 2002/198178. Compounds of formula Vb, wherein PG stands for a protecting group, preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons) and A is defined as —CH═CH— are known compounds and may be prepared according to literature procedures; starting from 4-methoxypyridine: Tetrahedron Lett. 2002, 43, 1779; J. Org. Chem. 2003, 68, 8867; Org. Lett. 2007, 9, 2871; [4+3] cycloaddition: Synlett 2003, 2175, J. Chem. Soc., Perkin Trans. I, 1992, 787-790. Compounds of formula Vb may be subjected to a deprotection reaction, e.g., by treatment with an acid, preferably 2,2,2-trifluoroacetic acid when PG is a tert-butoxycarbonyl group (see e.g., T. W. Greene et al. “Protective Groups in Organic Synthesis”, 3^{rd} edition 1999 by J. Wiley & Sons), leading to compounds of formula Vc, wherein A is defined as for formula I. Subsequent alkylation reaction with an alkylating reagent of general formula R²-LG, wherein R² is defined as in formula I and LG stands for a leaving group such as CI, Br, I, OMs, OTs or, OTf, in the presence of an organic or inorganic base, preferably K₂CO₃ or iPr₂NEt, then may furnish compounds of formula V. V can be converted into compound VI as described above. Alternatively, compounds of formula Vb may be converted into compounds of formula VIb, wherein PG stands for a protecting group, preferably a tert-butoxycarbonyl, ethoxycarbonyl or benzyloxycarbonyl group (see e.g., T. W. Greene et al. “Protective Groups in Organic Synthesis”, 3^{rd} edition 1999 by J. Wiley & Sons) and A is defined as for formula I, following known procedures described in WO9637494 and J. Org. Chem. 1977, 42, 3114.

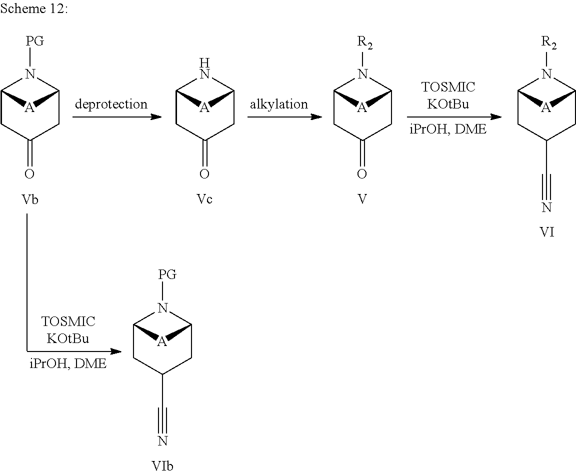

Certain intermediates of formula IIa, IIb, IIc, IId, IIe, IIf, IIg, IIh, IIi, IIj, IIm, IIn, IIo, IIp, II, IAa, IAb, IAc, IAd, IAe, IAf, III, IIa, IV, IVa, IVb, IBa, V, Vb, VI, and VIb are novel and as such form a further aspect of the invention. For example, certain novel intermediates include compounds of formula IIa, IIb, IIc, IId, IIe, IIf, IIg, IIh, IIi, IIj, IIm, IIn, IIo, IIp, II, IAa, IAb, IAc, IAd, IAe, IAf, III, IIa, IV, IVa, IVb, IBa, V, Vb, VI, and VIb wherein the substituents A, R1, R2, and Ra or R5 and R6, insofar as they are present, are as defined in Tables 1 to 208 above.

Agrochemically acceptable salts of the compounds of formula I are, for example, acid addition salts. Those salts are formed, for example, with strong inorganic acids, such as mineral acids, for example perchloric acid, sulfuric acid, nitric acid, nitrous acid, a phosphoric acid or a hydrohalic acid, with strong organic carboxylic acids, such as unsubstituted or substituted, for example halogen-substituted, C₁-C₄ alkanecarboxylic acids, for example formic acid, acetic acid or trifluoroacetic acid, unsaturated or saturated dicarboxylic acids, for example oxalic, malonic, succinic, maleic, fumaric or phthalic acid, hydroxycarboxylic acids, for example ascorbic, lactic, malic, tartaric or citric acid, or benzoic acid, or with organic sulfonic acids, such as unsubstituted or substituted, for example halogen-substituted, C₁-C₄ alkane- or aryl-sulfonic acids, for example methane- or p-toluene-sulfonic acid.

In order to apply an active ingredient (i.e. a compound of formula (I)) to insects (in particular neonicotinoid resistant insects) and/or crops of useful plants as required by the methods of the invention said active ingredient may be used in pure form or, more typically, formulated into a composition which includes, in addition to said active ingredient, a suitable inert diluent or carrier and optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). SFAs include non-ionic, cationic and/or anionic surfactants, as well as surfactant mixtures. Examples are suitable phosphates, such as salts of the phosphoric ester of a p-nonylphenol/(4-14)ethylene oxide adduct, or phospholipids. Further suitable phosphates are tris-esters of phosphoric acid with aliphatic or aromatic alcohols and/or bis-esters of alkyl phosphonic acids with aliphatic or aromatic alcohols, which are a high performance oil-type adjuvant. These tris-esters have been described, for example, in WO0147356, WO0056146, EP-A-0579052 or EP-A-1018299 or are commercially available under their chemical name. Preferred tris-esters of phosphoric acid for use in the new compositions are tris-(2-ethylhexyl) phosphate, tris-n-octyl phosphate and tris-butoxyethyl phosphate, where tris-(2-ethylhexyl) phosphate is most preferred. Suitable bis-ester of alkyl phosphonic acids are bis-(2-ethylhexyl)-(2-ethylhexyl)-phosphonate, bis-(2-ethylhexyl)-(n-octyl)-phosphonate, dibutyl-butyl phosphonate and bis(2-ethylhexyl)-tripropylene-phosphonate, where bis-(2-ethylhexyl)-(n-octyl)-phosphonate is particularly preferred.

The compositions according to the invention can preferably additionally include an additive comprising an oil of vegetable or animal origin, a mineral oil, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil such as ADIGOR® and MERO®, olive oil or sunflower oil, emulsified vegetable oil, such as AMIGO® (Rhône-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being important. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Also, alkoxylated fatty acids can be used as additives in the inventive compositions as well as polymethylsiloxane based additives, which have been described in WO08/037373.

Thus, in further embodiments according to any aspect of the invention mentioned hereinbefore, the compound of formula (I) will be in the form of a composition additionally comprising an agriculturally acceptable carrier or diluent.

It is preferred that all compositions (both solid and liquid formulations) comprise, by weight, 0.0001 to 95%, more preferably 1 to 85%, for example 5 to 60%, of a compound of formula (I). The composition is generally used for the control of pests such that a compound of formula (I) is applied at a rate of from 0.1 g to 10 kg per hectare, generally from 1 g to 6 kg per hectare, preferably 1 g to 2 kg per hectare, more preferably from 10 g to 1 kg per hectare, most preferably 10 g to 600 g per hectare.

When used in a seed dressing, a compound of formula (I) is generally used at a rate of 0.0001 g to 10 g (for example 0.001 g or 0.05 g), preferably 0.005 g to 10 g, more preferably 0.005 g to 4 g, per kilogram of seed.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallization in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurized, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

Preferred compositions for use in methods of the invention are composed in particular of the following constituents (throughout, percentages are by weight):

Emulsifiable concentrates (EC):
active ingredient: 1 to 90%, preferably 5 to 20%
SFA: 1 to 30%, preferably 10 to 20%
solvent: 5 to 98%, preferably 70 to 85%

### Dusts (DP):

active ingredient: 0.1 to 10%, preferably 0.1 to 1%
solid carrier/diluent: 99.9 to 90%, preferably 99.9 to 99%
Suspension concentrates (SC):
active ingredient: 5 to 75%, preferably 10 to 50%
water: 94 to 24%, preferably 88 to 30%
SFA: 1 to 40%, preferably 2 to 30%
Wettable powders (WP):
active ingredient: 0.5 to 90%, preferably 1 to 80%, more preferably 20 to 30%
SFA: 0.5 to 20%, preferably 1 to 15%
solid carrier: 5 to 99%, preferably 15 to 98%

### Granules (GR, SG, WG):

active ingredient: 0.5 to 60%, preferably 5 to 60%, more preferably 50 to 60%
solid carrier/diluent: 99.5 to 40%, preferably 95 to 40%, more preferably 50 to 40%

A compound of formula I may be applied to a pest or crop of useful plants using any standard application method with which the skilled man is familiar, such as foliar spay or treatment of the plant propagation materials of the crop. Similarly, for methods of controlling insect resistance, neonicotinoid insecticides may be applied to an insect/crop/plant propagation material of useful plants using any known method of application. Further guidance may be found in the art, which includes for example, advice on application given on the labels of commercially available products.

In another aspect of the invention, the neonicotinoid insecticide is applied to the plant propagation material (such as seeds, young plants, transplants etc.) of the respective crops followed by the foliar application of a compound of the formula (I) starting in the 3- to 5-leaf up to the fruit setting crop stage. It has been found, that beginning with the 3- to 5-leaf crop stage, when the level of insect control by the neonicotinoid insecticide starts to decrease, another boost in insect control can be achieved by the foliar application of a compound of the formula (I), which, surprisingly, is accompanied by pronounced crop enhancement effects such as an increase in the formation of fine roots, synchronisation of flowering, drought resistance and, in particular, an increase in yield.

Examples of typical formulations are provided below (throughout, percentages are by weight)

| Example F1: Solutions | a) | b) | c) | d) |
|---|---|---|---|---|
| active ingredient | 80% | 10% | 5% | 95% |
| ethylene glycol monomethyl ether | 20% | — | — | — |
| polyethylene glycol (mol. wt 400) | — | 70% | — | — |
| N-methyl-2-pyrrolidone | — | 20% | — | — |
| epoxidised coconut oil | — | — | 1% | 5% |
| petroleum fraction (boiling range 160-190. | — | — | 94% | — |
| degree.) | | | | |

These solutions are suitable for application in the form of micro-drops.

| Example F2: Granules | a) | b) | c) | d) |
|---|---|---|---|---|
| active ingredient | 5% | 10% | 8% | 21% |
| Kaolin | 94% | — | 79% | 54% |
| Highly dispersed silicic acid | 1% | — | 13% | 7% |
| Attapulgite | — | 90% | — | 18% |

The active ingredient is dissolved in dichloromethane, the solution is sprayed onto the carrier, and the solvent is subsequently evaporated off in vacuo.

| Example F3: Dusts | a) | b) |
|---|---|---|
| active ingredient | 2% | 5% |
| Highly dispersed silicic acid | 1% | 5% |
| Talcum | 97% | — |
| Kaolin | — | 90% |

Ready-for-use dusts are obtained by intimately mixing the carriers with the active ingredient.

| Example F4: Wettable powders | |
|---|---|
| active ingredient | 25% |
| Sodium sulphate | 5% |
| castor oil polyethylene glycol ether (36-37 mol of ethylene oxide) | 10% |
| silicone oil | 1% |
| Agridex | 2% |
| highly dispersed silicic acid | 10% |
| kaolin powder | 37% |
| sulfite spent lye powder | 5% |
| Ultravon W-300% (disodium salt of 1-benzyl-2- | 5% |
| heptadecylbenzimidazole-X,X′-disulfonic acid) | |

The active ingredient is mixed with the other formulation components and the mixture is ground in a suitable mill, affording wettable powders which can be diluted with water to give suspensions of the desired concentration.

| Example F5: Dusts | a) | b) |
|---|---|---|
| active ingredient | 5% | 8% |
| Talcum | 95% | — |
| Kaolin | — | 92% |

Ready-for-use dusts are obtained by mixing the active ingredient with the carrier and grinding the mixture in a suitable mill.

| Example F6: Extruder granules | |
|---|---|
| active ingredient | 10% |
| Sodium lignosulfonate | 2% |
| Carboxymethylcellulose | 1% |
| Kaolin | 87% |

The active ingredient is mixed and ground with the other formulation components, and the mixture is subsequently moistened with water. The moist mixture is extruded and granulated and then the granules are dried in a stream of air.

| Example F7: Coated granules | |
|---|---|
| active ingredient | 3% |
| Polyethylene glycol (mol. wt. 200) | 3% |
| Kaolin | 94% |

The finely ground active ingredient is uniformly applied, in a mixer, to the kaolin moistened with polyethylene glycol. Non-dusty coated granules are obtained in this manner.

| Example F8: Suspension concentrate | |
|---|---|
| active ingredient | 40% |
| Ethylene glycol | 10% |
| Nonylphenol polyethylene glycol | 6% |
| Ether (15 mol of ethylene oxide) | |
| Sodium lignosulfonate | 10% |
| Carboxymethylcellulose | 1% |
| Aqueous formaldehyde solution (37%) | 0.2% |
| Aqueous silicone oil emulsion (75%) | 0.8% |
| Water | 32% |

The finely ground active ingredient is intimately mixed with the other formulation components giving a suspension concentrate from which suspensions of any desired concentration can be obtained by dilution with water.

| Example F9: Emulsifiable concentrates | a) | b) | c) |
|---|---|---|---|
| active ingredient | 25% | 40% | 50% |
| Calcium dodecylbenzenesulfonate | 5% | 8% | 6% |
| Castor oil polyethylene glycol ether (36 mol of ethylene | 5% | — | — |
| oxide) | | | |
| Tristyrylphenol polyethylene glycol ether (30 mol of | — | 12% | 4% |
| ethylene oxide | | | |
| Cyclohexanone | — | 15% | 20% |
| Xylene mixture | 65% | 25% | 20% |

Emulsions of any desired concentration can be produced from such concentrates by dilution with water.

| Example F10: Wettable powders | a) | b) | c) |
|---|---|---|---|
| active ingredient | 25% | 50% | 75% |
| Sodium lignosulfonate | 5% | 5% | — |
| Sodium laurylsulfate | 3% | — | 5% |
| Sodium diisobutylnapthalene-sulfonate | — | 6% | 10% |
| Octylphenol polyethylene glycol ether (7-8 mol of | — | 2% | — |
| ethylene oxide) | | | |
| Highly dispersed silicic acid | 5% | 10% | 10% |
| Kaolin | 62% | 27% | — |

The active ingredient is mixed with the other formulation components and the mixture is ground in a suitable mill, affording wettable powders which can be diluted with water to give suspensions of the desired concentration.

| Example F11: Emulsifiable concentrate | |
|---|---|
| active ingredient | 10% |
| Octylphenol polyethylene glycol ether (4-5 mol of ethylene oxide) | 3% |
| Calcium dodecylbenzenesulfonate | 3% |
| Castor oil polyglycol ether (36 mol of ethylene oxide) | 4% |
| Cyclohexanone | 30% |
| Xylene mixture | 50% |

Emulsions of any required concentration can be obtained from this concentrate by dilution with water.

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulfuric acid (for example sodium lauryl sulfate), salts of sulfonated aromatic compounds (for example sodium dodecylbenzenesulfonate, calcium dodecylbenzenesulfonate, butylnaphthalene sulfonate and mixtures of sodium di-isopropyl- and tri-isopropyl-naphthalene sulfonates), ether sulfates, alcohol ether sulfates (for example sodium laureth-3-sulfate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulfosuccinamates, paraffin or olefine sulfonates, taurates and lignosulfonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilizers (for example nitrogen-, potassium- or phosphorus-containing fertilizers). Suitable formulation types include granules of fertilizer. The mixtures preferably contain up to 25% by weight of the compound of formula (I).

The invention therefore also provides a fertilizer composition comprising a fertilizer and a compound of formula (I).

The compositions of this invention may contain other compounds having biological activity, for example micronutrients or compounds having fungicidal activity or which possess plant growth regulating, herbicidal, insecticidal, nematicidal or acaricidal activity.

The compound of formula (I) may be the sole active ingredient of the composition or it may be admixed with one or more additional active ingredients such as a pesticide, e.g. a insecticide, fungicide or herbicide, or a synergist or plant growth regulator where appropriate. An additional active ingredient may provide a composition having a broader spectrum of activity or increased persistence at a locus; synergize the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin and gamma cyhalothrin), bifenthrin, fenpropathrin, cyfluthrin, tefluthrin, fish safe pyrethroids (for example ethofenprox), natural pyrethrin, tetramethrin, S-bioallethrin, fenfluthrin, prallethrin, acrinathirin, etofenprox or 5-benzyl-3-furylmethyl-(E)-(1R,3S)-2,2-dimethyl-3-(2-oxothiolan-3-ylidenemethyl)cyclopropane carboxylate;

b) Organophosphates, such as profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;

c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;

d) Benzoyl ureas, such as diflubenzuron, triflumuron, hexaflumuron, flufenoxuron, diafenthiuron, lufeneron, novaluron, noviflumuron or chlorfluazuron;

e) Organic tin compounds, such as cyhexatin, fenbutatin oxide or azocyclotin;

f) Pyrazoles, such as tebufenpyrad, tolfenpyrad, ethiprole, pyriprole, fipronil, and fenpyroximate;

g) Macrolides, such as avermectins or milbemycins, for example abamectin, emamectin benzoate, ivermectin, milbemycin, spinosad, azadirachtin, milbemectin, lepimectin or spinetoram;

h) Hormones or pheromones;

i) Organochlorine compounds, such as endosulfan (in particular alpha-endosulfan), benzene hexachloride, DDT, chlordane or dieldrin;

j) Amidines, such as chlordimeform or amitraz;

k) Fumigant agents, such as chloropicrin, dichloropropane, methyl bromide or metam;

l) Neonicotinoid compounds, such as imidacloprid, thiacloprid, acetamiprid, nitenpyram, dinotefuran, thiamethoxam, clothianidin, or nithiazine;

m) Diacylhydrazines, such as tebufenozide, chromafenozide or methoxyfenozide;

n) Diphenyl ethers, such as diofenolan or pyriproxifen;

o) Pyrazolines such as Indoxacarb or metaflumizone;

p) Ketoenols, such as Spirotetramat, spirodiclofen or spiromesifen;

q) Diamides, such as flubendiamide, chlorantraniliprole (Rynaxypyr®) or cyantraniliprole;

r) Essential oils such as Bugoil®—(PlantImpact); or

s) a compound selected from buprofezine, flonicamid, acequinocyl, bifenazate, cyenopyrafen, cyflumetofen, etoxazole, flometoquin, fluacrypyrim, fluensulfone, flufenerim, flupyradifuone, harpin, iodomethane, dodecadienol, pyridaben, pyridalyl, pyrimidifen, flupyradifurone, 4-[(6-Chloro-pyridin-3-ylmethyl)-(2,2-difluoro-ethyl)-amino]-5H-furan-2-one (DE 102006015467), CAS: 915972-17-7 (WO 2006129714; WO2011/147953; WO2011/147952), CAS: 26914-55-8 (WO 2007020986), chlorfenapyr, pymetrozine, sulfoxaflor and pyrifluqinazon.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulfonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704) (e.g. acibenzolar-S-methyl), alanycarb, aldimorph, anilazine, azaconazole, azoxystrobin, benalaxyl, benomyl, benthiavalicarb, biloxazol, bitertanol, bixafen, blasticidin S, boscalid, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulfate, copper tallate and Bordeaux mixture, cyclufenamid, cymoxanil, cyproconazole, cyprodinil, debacarb, di-2-pyridyl disulfide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl-(Z)—N-benzyl-N-([methyl(methyl-thioethylideneaminooxycarbonyl)amino]thio)-β-alaninate, etridiazole, famoxadone, fenamidone (RPA407213), fenarimol, fenbuconazole, fenfuram, fenhexamid (KBR²738), fenpiclonil, fenpropidin, fenpropimorph, fentin acetate, fentin hydroxide, ferbam, ferimzone, fluazinam, fludioxonil, flumetover, fluopyram, fluoxastrobin, fluoroimide, fluquinconazole, flusilazole, flutolanil, flutriafol, fluxapyroxad, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, isopyrazam, kasugamycin, kresoxim-methyl, LY186054, LY211795, LY248908, mancozeb, mandipropamid, maneb, mefenoxam, metalaxyl, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, penflufen, penthiopyrad, phenazin oxide, phosetyl-AI, phosphorus acids, phthalide, picoxystrobin (ZA1963), polyoxinD, polyram, probenazole, prochloraz, procymidone, propamocarb, propiconazole, propineb, propionic acid, prothioconazole, pyrazophos, pyrifenox, pyrimethanil, pyraclostrobin, pyroquilon, pyroxyfur, pyrrolnitrin, quaternary ammonium compounds, quinomethionate, quinoxyfen, quintozene, sedaxane, sipconazole (F-155), sodium pentachlorophenate, spiroxamine, streptomycin, sulfur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram, N-[9-(dichloromethylene)-1,2,3,4-tetrahydro-1,4-methanonaphthalen-5-yl]-3-(difluoromethyl)-1-methyl-1H-pyrazole-4-carboxamide [1072957-71-1], 1-methyl-3-difluoromethyl-1H-pyrazole-4-carboxylic acid (2-dichloromethylene-3-ethyl-1-methyl-indan-4-yl)-amide, and 1-methyl-3-difluoromethyl-4H-pyrazole-4-carboxylic acid [2-(2,4-dichloro-phenyl)-2-methoxy-1-methyl-ethyl]amide.

In addition, biological agents may be included in the composition of the invention e.g. Baciullus species such as Bacillus firmus, Bacillus cereus, Bacillus subtilis, and Pasteuria species such as Pasteuria penetrans and Pasteuria nishizawae. A suitable Bacillus firmus strain is strain CNCM 1-1582 which is commercially available as BioNem™. A suitable Bacillus cereus strain is strain CNCM 1-1562. Of both Bacillus strains more details can be found in U.S. Pat. No. 6,406,690. Other biological organisms that may be included in the compositions of the invention are bacteria such as Streptomyces spp. such as S. avermitilis, and fungi such as Pochonia spp. such as P. chlamydosporia. Also of interest are Metarhizium spp. such as M. anisopliae; Pochonia spp. such as P. chlamydosporia.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™

The following mixtures of the compounds of formula I with active ingredients are preferred (the abbreviation “TX” means “one compound selected from the group consisting of the compounds described in Tables 1 to 208 (above) of the present invention”):

an adjuvant selected from the group of substances consisting of petroleum oils (alternative name) (628)+TX, an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos (alternative name) [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate (alternative name)+TX, bromo-cyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben (alternative name)+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chino-methionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin 11 (696)+TX, cinerins (696)+TX, clofentezine (158)+TX, closantel (alternative name) [CCN]+TX, coumaphos (174)+TX, crotamiton (alternative name) [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafen-thiuron (226)+TX, dialifos (1042)+TX, diazinon (227)+TX, dichlofluanid (230)+TX, dichlorvos (236)+TX, dicliphos (alternative name)+TX, dicofol (242)+TX, dicrotophos (243)+TX, dienochlor (1071)+TX, dimefox (1081)+TX, dimethoate (262)+TX, dinactin (alternative name) (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram (alternative name) [CCN]+TX, disulfoton (278)+TX, DNOC (282)+TX, dofenapyn (1113)+TX, doramectin (alternative name) [CCN]+TX, endosulfan (294)+TX, endothion (1121)+TX, EPN (297)+TX, eprinomectin (alternative name) [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad (alternative name)+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (alternative name) (473)+TX, isopropyl 0-(methoxyaminothiophosphoryl)salicylate (IUPAC name) (473)+TX, ivermectin (alternative name) [CCN]+TX, jasmolin 1 (696)+TX, jasmolin 11 (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen (alternative name) [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime (alternative name) [CCN]+TX, mipafox (1293)+TX, monocrotophos (561)+TX, morphothion (1300)+TX, moxidectin (alternative name) [CCN]+TX, naled (567)+TX, NC-184 (compound code)+TX, NC-512 (compound code)+TX, nifluridide (1309)+TX, nikkomycins (alternative name) [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (alternative name) (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (alternative name) (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos (alternative name)+TX, selamectin (alternative name) [CCN]+TX, SI-0009 (compound code)+TX, sophamide (1402)+TX, spirodiclofen (738)+TX, spiromesifen (739)+TX, SSI-121 (development code) (1404)+TX, sulfiram (alternative name) [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam (alternative name)+TX, tetrachlorvinphos (777)+TX, tetradifon (786)+TX, tetranactin (alternative name) (653)+TX, tetrasul (1425)+TX, thiafenox (alternative name)+TX, thiocarboxime (1431)+TX, thiofanox (800)+TX, thiometon (801)+TX, thioquinox (1436)+TX, thuringiensin (alternative name) [CCN]+TX, triamiphos (1441)+TX, triarathene (1443)+TX, triazophos (820)+TX, triazuron (alternative name)+TX, trichlorfon (824)+TX, trifenofos (1455)+TX, trinactin (alternative name) (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,
an anthelmintic selected from the group of substances consisting of abamectin (1)+TX, crufomate (1011)+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX,
an avicide selected from the group of substances consisting of chloralose (127)+TX, endrin (1122)+TX, fenthion (346)+TX, pyridin-4-amine (IUPAC name) (23) and strychnine (745)+TX,
a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX,
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX,
a soil sterilant selected from the group of substances consisting of iodomethane (IUPAC name) (542) and methyl bromide (537)+TX,
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX,
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure Ill (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) and trunc-call (alternative name) [CCN]+TX,
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX,
an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichloro-phenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethyl-carbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion (alternative name) [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin (alternative name) [CCN]+TX, allyxycarb (866)+TX, alpha-cypermethrin (202)+TX, alpha-ecdysone (alternative name) [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (alternative name) (41)+TX, azamethiphos (42)+TX, azinphos-ethyl (44)+TX, azinphos-methyl (45)+TX, azothoate (889)+TX, Bacillus thuringiensis delta endotoxins (alternative name) (52)+TX, barium hexafluorosilicate (alternative name) [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (alternative name) (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate (alternative name)+TX, bromfenvinfos (914)+TX, bromocyclen (918)+TX, bromo-DDT (alternative name) [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben (alternative name)+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (alternative name) (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin (alternative name)+TX, cismethrin (80)+TX, clocythrin (alternative name)+TX, cloethocarb (999)+TX, closantel (alternative name) [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton (alternative name) [CCN]+TX, crotoxyphos (1010)+TX, crufomate (1011)+TX, cryolite (alternative name) (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate (alternative name) [CCN]+TX, d-limonene (alternative name) [CCN]+TX, d-tetramethrin (alternative name) (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos (alternative name)+TX, dicresyl (alternative name) [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor (alternative name) [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin (alternative name) [CCN]+TX, DSP (1115)+TX, ecdysterone (alternative name) [CCN]+TX, EI 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin (alternative name) [CCN]+TX, esfenvalerate (302)+TX, etaphos (alternative name) [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (alternative name) (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad (alternative name)+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (alternative name) (473)+TX, isodrin (1235)+TX, isofenphos (1236)+TX, isolane (1237)+TX, isoprocarb (472)+TX, isopropyl O-(methoxyaminothiophosphoryl)salicylate (IUPAC name) (473)+TX, isoprothiolane (474)+TX, isothioate (1244)+TX, isoxathion (480)+TX, ivermectin (alternative name) [CCN]+TX, jasmolin I (696)+TX, jasmolin II (696)+TX, jodfenphos (1248)+TX, juvenile hormone I (alternative name) [CCN]+TX, juvenile hormone II (alternative name) [CCN]+TX, juvenile hormone III (alternative name) [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (alternative name) (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (alternative name) (533)+TX, methoxychlor (534)+TX, methoxyfenozide (535)+TX, methyl bromide (537)+TX, methyl isothiocyanate (543)+TX, methylchloroform (alternative name) [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime (alternative name) [CCN]+TX, mipafox (1293)+TX, mirex (1294)+TX, monocrotophos (561)+TX, morphothion (1300)+TX, moxidectin (alternative name) [CCN]+TX, naftalofos (alternative name) [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron (alternative name) [CCN]+TX, pentachlorophenol (623)+TX, pentachlorophenyl laurate (IUPAC name) (623)+TX, permethrin (626)+TX, petroleum oils (alternative name) (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I (alternative name) [CCN]+TX, precocene II (alternative name) [CCN]+TX, precocene Ill (alternative name) [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia (alternative name) [CCN]+TX, quinalphos (711)+TX, quinalphos-methyl (1376)+TX, quinothion (1380)+TX, quintiofos (1381)+TX, R-1492 (development code) (1382)+TX, rafoxanide (alternative name) [CCN]+TX, resmethrin (719)+TX, rotenone (722)+TX, RU 15525 (development code) (723)+TX, RU 25475 (development code) (1386)+TX, ryania (alternative name) (1387)+TX, ryanodine (traditional name) (1387)+TX, sabadilla (alternative name) (725)+TX, schradan (1389)+TX, sebufos (alternative name)+TX, selamectin (alternative name) [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (alternative name) (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam (alternative name)+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox (alternative name)+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin (alternative name) [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron (alternative name)+TX, trichlorfon (824)+TX, trichlormetaphos-3 (alternative name) [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (alternative name) (725)+TX, veratrine (alternative name) (725)+TX, XMC (853)+TX, xylylcarb (854)+TX, YI-5302 (compound code)+TX, zeta-cypermethrin (205)+TX, zetamethrin (alternative name)+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX,
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX,
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX,
a nitrification inhibitor selected from the group of substances consisting of potassium ethylxanthate [CCN] and nitrapyrin (580)+TX,
a plant activator selected from the group of substances consisting of acibenzolar (6)+TX, acibenzolar-S-methyl (6)+TX, probenazole (658) and Reynoutria sachalinensis extract (alternative name) (720)+TX,
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,
a virucide selected from the group of substances consisting of imanin (alternative name) [CCN] and ribavirin (alternative name) [CCN]+TX,
a wound protectant selected from the group of substances consisting of mercuric oxide (512)+TX, octhilinone (590) and thiophanate-methyl (802)+TX,
and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiaben-dazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzolar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-L190 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (dislosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual-A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright© 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula I selected from Tables 1 to 208 (above) with active ingredients described above comprises a compound selected from Tables 1 to 208 (above) and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables 1 to 208 (above) and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables 1 to 208 (above) and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is preferably 1 to 2000 g of active ingredient per hectare, more preferably 10 to 1000 g/ha, most preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

### EXAMPLES

The following Examples illustrate, but do not limit, the invention.

The following abbreviations were used in this section: DMF: dimethylformamide; THF: tetrahydrofuran; EtOAc: ethyl acetate; s=singlet; bs=broad singlet; d=doublet; dd=double doublet; dt=double triplet; t=triplet, tt=triple triplet, q=quartet, sept=septet; m=multiplet; Me=methyl; Et=ethyl; Pr=propyl; Bu=butyl; M.p.=melting point; RT=retention time, [M+H]⁺=molecular mass of the molecular cation, [M−H]⁻=molecular mass of the molecular anion.

The following LC-MS methods were used to characterize the compounds:

### Method G:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 1.2-1.5 min 100% B; Flow (ml/min) 0.85

### Method H:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 2.7-3.0 min 100% B; Flow (ml/min) 0.85

### Method I:

Spectra were recorded on a Mass Spectrometer from Agilent (QQQ6410 mass spectrometer) equipped with an electrospray source (Polarity: positive or negative ions, Capillary: 4.00 kV, Desolvation Temperature: 350° C., Cone Gas Flow: 11 L/h, Mass range: 100 to 900 Da) and an Agilent 1200 from Agilent: Quaternary pump, heated column compartment and diode-array detector. Column: Acquity BEH, C18, 1.7 μm, 30×2.1 mm, Temp: 25° C., DAD Wavelength range (nm): 210 to 400, Solvent Gradient: A=water+0.05% HCOOH, B=Acetonitrile+0.05% HCOOH: gradient: gradient: 0 min 10% B, 90% A; 2.0-3.0 min 100% B; 3-4 10% B Flow (mL/min) 1.8 mL/min.

### PREPARATION EXAMPLES

### Example P1

### Preparation of tert-butyl (1 S,5R)-3-(5-bromo-3-pyridyl)-3-carbamothioyl-8-azabicyclo[3.2.1]octane-8-carboxylate (Compound E.021)

### Step 1: Preparation of (1S,5R)-3-cyano-8-aza-bicyclo[3.2.1]octane-8-carboxylic acid tert-butyl ester

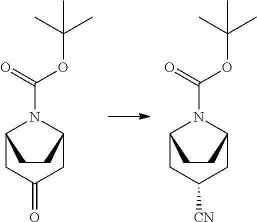 [SMILES: CC(C)(C)OC(=O)N1[C@@H]2CC[C@H]1CC(=O)C2 ; CC(C)(C)OC(=O)N1[C@@H]2CC[C@H]1C[C@H](C#N)C2]

Potassium tert-butoxide (6.23 g, 55.5 mmol) was suspended at 0° C. in 1,2-dimethoxyethane (DME) (15 mL) under argon. Then, within 30 min, a solution of tosylmethyl isocyanide (6.50 g, 33.3 mmol) in DME (20 mL) was added dropwise while keeping the temperature below 5° C. The reaction mixture became immediately brown and was stirred for additional 1 h at 0° C. Then, isopropanol (3.4 mL, 44.6 mmol) was added dropwise at 0° C. The reaction mixture was stirred for additional 30 min, before dropwise addition of (1 S,5R)-3-oxo-8-azabicyclo[3.2.1]octane-8-carboxylic acid tert-butyl ester (5.00 g, 22.2 mmol) (prepared according to Berdini et al., Tetrahedron 2002, 58, 5669) were within 30 minutes maintaining the reaction temperature below 5° C. After completion of the addition, stirring was continued for 1 h at 0° C. and then allowed to warm to room temperature overnight. The reaction mixture was filtered over Celite and the residue was intensively washed with solvent. The organic layers were combined and evaporated to give the crude product. The crude material was purified by flash chromatography (ethyl acetate/cyclohexane) to afford (1 S,5R)-3-cyano-8-aza-bicyclo[3.2.1]octane-8-carboxylic acid tert-butyl ester as a white solid (m.p. 97-98° C.).

¹H NMR (CDCl₃, TMS) δ/ppm: 1.48 (s, 9H), 1.62 (m, 2H), 1.85 (m, 2H), 1.95-2.10 (br m, 4H), 2.90-3.05 (m, 1H), 4.15-4.35 (br s, 2H).

### Step 2: Preparation of (1S,5R)-3-(5-bromo-pyridin-3-yl)-3-cyano-8-aza-bicyclo[3.2.1]octane-8-carboxylic acid tert-butyl ester

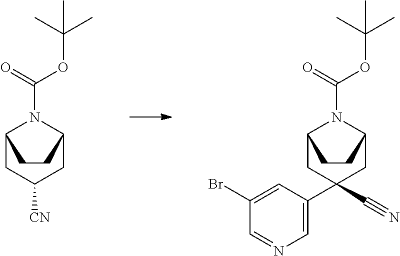 [SMILES: CC(C)(C)OC(=O)N1[C@@H]2CC[C@H]1C[C@H](C#N)C2 ; CC(C)(C)OC(=O)N1[C@@H]2CC[C@H]1C[C@](C#N)(c1cncc(Br)c1)C2]

Lithium bis(trimethylsilyl)amide (46.75 mL of a 1M solution in THF) was added dropwise to a stirred solution of (1S,5R)-3-cyano-8-aza-bicyclo[3.2.1]octane-8-carboxylic acid tert-butyl ester (10.0 g, 42.5 mmol) and 3-bromo-5-fluoro-pyridine (7.85 g, 44.6 mmol) in tetrahydrofuran (THF) (100 mL) at room temperature over 1 h under argon atmosphere. The reaction mixture turned immediately brown. Stirring was continued at room temperature for 20 h. The reaction mixture was poured into cold water and extracted with ethyl acetate (3×). The combined extracts were washed with brine, dried (MgSO₄) and evaporated under reduced pressure to give a brown oil. Purification by flash chromatography (SiO₂, 10 to 70% ethyl acetate/cyclohexane) furnished (1 S,5R)-3-(5-bromo-pyridin-3-yl)-3-cyano-8-aza-bicyclo[3.2.1]octane-8-carboxylic acid tert-butyl ester as a white solid.

¹H NMR (CDCl₃, TMS) δ/ppm: 1.50 (s, 9H), 2.10-2.21 (m, 2H), 2.22-2.35 (br m, 3H), 2.35-2.45 (br m, 3H), 4.30-4.52 (br m, 2H), 7.90 (t, 1H), 8.65 (2 d, 2H).

### Step 3: Preparation of tert-butyl (1 S,5R)-3-(5-bromo-3-pyridyl)-3-carbamothioyl-8-azabicyclo[3.2.1]octane-8-carboxylate (Compound E.021)

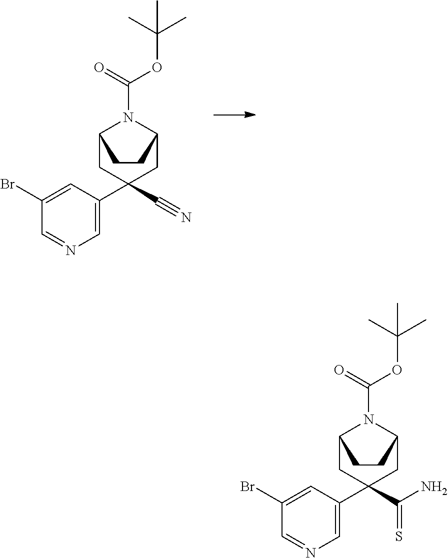 [SMILES: CC(C)(C)OC(=O)N1[C@@H]2CC[C@H]1C[C@](C#N)(c1cncc(Br)c1)C2 ; CC(C)(C)OC(=O)N1[C@@H]2CC[C@H]1C[C@](C(N)=S)(c1cncc(Br)c1)C2]

To a solution of (1S,3S,5R)-3-(5-bromo-pyridin-3-yl)-3-cyano-8-aza-bicyclo[3.2.1]octane-8-carboxylic acid tert-butyl ester (2.05 g, 5.21 mmol) in pyridine (21 mL) at 20° C. was added dropwise a solution of ammonium polysulfide (3.34 mL, 20.8 mmol) and the mixture was stirred at 20° C. for 24 h. After complexion of the reaction, the mixture was poured into ice water (0° C.) to form a precipitate. The solid was filtered, washed with water (2×) and dried under vacuum. The crude product was then stirred in dichloromethane, filtered and dried under vacuum to give tert-butyl (1 S,5R)-3-(5-bromo-3-pyridyl)-3-carbamothioyl-8-azabicyclo[3.2.1]octane-8-carboxylate as an off-white powder (m.p. 228-230° C.). An additional portion of the product was isolated from the dichloromethane mother liquid through concentration.

UPLC MS (method G): RT 0.98 min. m/z 426 [M+H]⁺.

¹H NMR (DMSO-d₆, TMS) δ/ppm: 1.32 (s, 9H), 1.68-1.80 (br s, 2H), 1.90-2.10 (br s, 2H), 3.50-3.65 (br d, 2H), 4.08-4.18 (br s, 2H), 7.96 (t, 1H), 8.57 (d, 1H), 8.59 (d, 1H), 9.31 (br s, 1H), 9.95 (br s, 1H).

### Example P2

### Preparation of (1R,5S)-3-(5-bromo-3-pyridyl)-8-azabicyclo[3.2.1]octane-3-carbothioamide (Compound E.010)

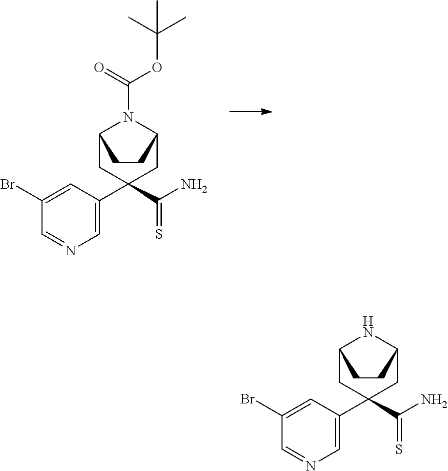 [SMILES: CC(C)(C)OC(=O)N1[C@@H]2CC[C@H]1C[C@](C(N)=S)(c1cncc(Br)c1)C2 ; NC(=S)[C@@]1(c2cncc(Br)c2)C[C@H]2CC[C@@H](C1)N2]

To a suspension of tert-butyl (1 S,5R)-3-(5-bromo-3-pyridyl)-3-carbamothioyl-8-azabicyclo[3.2.1]octane-8-carboxylate (200 mg, 0.470 mmol) in dichloromethane (1.2 mL) at room temperature was slowly added trifluoroacetic acid (0.11 mL, 1.41 mmol). The reaction mixture became a clear solution and was stirred at 20° C. for 4 days. The reaction mixture was concentrated and the residue (TFA-salt) was suspended in saturated aqueous Na₂CO₃ and vigorously stirred for 4 h. The suspension was filtered and the solid was dried under vacuum to give (1R,5S)-3-(5-bromo-3-pyridyl)-8-azabicyclo[3.2.1]-octane-3-carbothioamide as off-white powder (m.p. 164-168° C.).

UPLC MS (method G): RT 0.46 min. m/z 326 [M+H]⁺.

¹H NMR (DMSO-d₆, TMS) δ/ppm: 1.40-1.60 (m, 2H), 1.80-2.05 (m, 4H), 3.48 (s, 2H), 3.40-3.60 (m, 2H), 7.95 (br s, 1H), 8.52 (d, 1H), 8.59 (d, 1H), 9.20 (br s, 1H), 9.85 (br s, 1H).

### Example P3

### Preparation of (1S,5R)-3-(5-bromo-3-pyridyl)-8-(2,2,2-trifluoroethyl)-8-azabicyclo[3.2.1]octane-3-carbothioamide (Compound E.007)

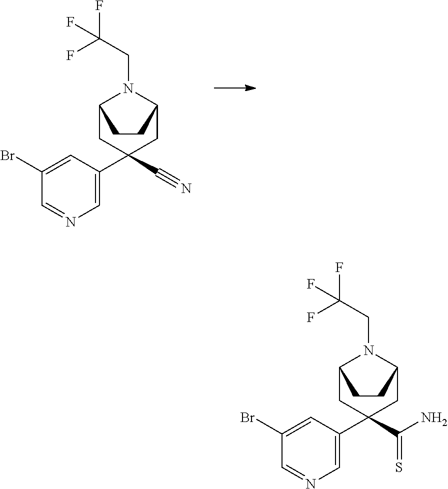 [SMILES: N#C[C@@]1(c2cncc(Br)c2)C[C@H]2CC[C@@H](C1)N2CC(F)(F)F ; NC(=S)[C@@]1(c2cncc(Br)c2)C[C@H]2CC[C@@H](C1)N2CC(F)(F)F]

To a solution of (1S,5R)-3-(5-bromo-3-pyridyl)-8-(2,2,2-trifluoroethyl)-8-azabicyclo-[3.2.1]octane-3-carbonitrile (0.10 g, 0.27 mmol) (prepared according to WO 96/37494) in pyridine (0.2 mL) at 20° C. was added dropwise a solution of ammonium polysulfide (0.18 mL, 1.07 mmol) and the mixture was stirred at 20° C. for 24 h. After completion of the reaction, the mixture was poured into ice water to form a precipitate. The solid was filtered, washed with water (2×) and dried under vacuum. The crude product was then purified by trituration in dichloromethane, filtered and dried to furnish (1 S,5R)-3-(5-bromo-3-pyridyl)-8-(2,2,2-trifluoroethyl)-8-azabicyclo[3.2.1]octane-3-carbothioamide as an off-white powder (m.p. 228-230° C.).

UPLC MS (method G): RT 0.94 min. m/z 408 [M+H]⁺.

¹H NMR (DMSO-d₆, TMS) δ/ppm: 1.60-1.80 (m, 2H), 1.85-2.00 (m, 2H), 2.07 (dd, 2H), 3.02 (dd, 2H), 3.28 (br s, 2H), 3.51 (dd, 2H), 7.95 (t, 1H), 8.54 (d, 1H), 8.58 (d, 1H), 9.26 (br s, 1H), 9.88 (br s, 1H).

### Example P4

### Preparation of (1S,5R)-3-(5-cyano-3-pyridyl)-8-(2,2,2-trifluoroethyl)-8-azabicyclo[3.2.1]octane-3-carbothioamide (Compound E.009)

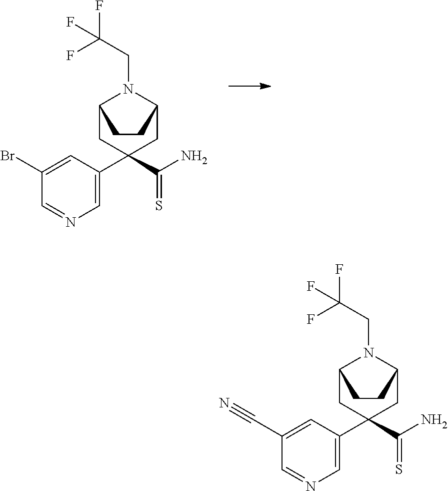 [SMILES: NC(=S)[C@@]1(c2cncc(Br)c2)C[C@H]2CC[C@@H](C1)N2CC(F)(F)F ; N#Cc1cncc([C@]2(C(N)=S)C[C@H]3CC[C@@H](C2)N3CC(F)(F)F)c1]

A mixture of (1S,5R)-3-(5-bromo-3-pyridyl)-8-(2,2,2-trifluoroethyl)-8-azabicyclo[3.2.1]octane-3-carbothioamide (835 mg, 2.05 mmol), Zn powder (16 mg, 0.24 mmol), Zn(CN)₂ (147 mg, 1.23 mmol), and 1,1′-bis(diphenylphosphino)ferrocene (46.8 mg, 0.082 mmol) were suspended in N,N-dimethyl acetamide (7.6 mL) at room temperature under argon atmosphere. After continuous purging with argon for 20 min, Pd₂(dba)₃ (38.6 mg, 0.041 mmol) was added to give a light yellow suspension. The reaction mixture was heated to 135° C. and stirred for 30 min. After completion of the reaction, the mixture was cooled to room temperature and quenched with aqueous ammonia (15 mL of a 2M solution), filtered through Celite and extracted with ethyl acetate. The combined organic layers were washed with brine, dried (MgSO₄), filtered and evaporated in vacuo. The crude product was purified by flash chromatography (SiO₂, 0 to 70% ethyl acetate/heptane) to furnish (1 S,5R)-3-(5-cyano-3-pyridyl)-8-(2,2,2-trifluoroethyl)-8-azabicyclo[3.2.1]octane-3-carbothioamide as a light yellow powder (m.p. 180-186° C.).

UPLC MS (method G): RT 0.84 min. m/z 355 [M+H]⁺.

### Example P5

### Preparation of (1S,5R)-3-(5-chloro-3-pyridyl)-8-(2,2,2-trifluoroethyl)-8-azabicyclo[3.2.1]octane-3-carbothioamide (Compound E.002)

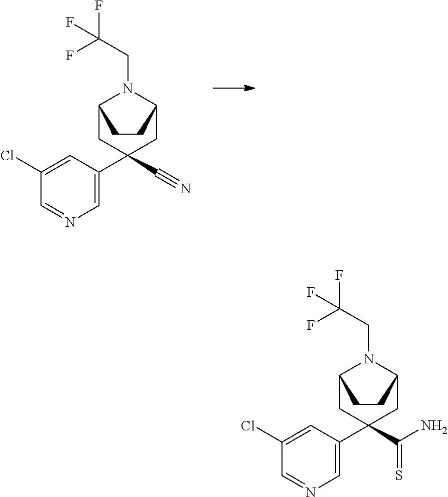 [SMILES: N#C[C@@]1(c2cncc(Cl)c2)C[C@H]2CC[C@@H](C1)N2CC(F)(F)F ; NC(=S)[C@@]1(c2cncc(Cl)c2)C[C@H]2CC[C@@H](C1)N2CC(F)(F)F]

A round-bottom flask charged with a solution of (1S,5R)-3-(5-chloro-3-pyridyl)-8-(2,2,2-trifluoroethyl)-8-azabicyclo[3.2.1]octane-3-carbonitrile (1.50 g, 4.50 mmol) (prepared according to WO 96/37494) in 1,4-dioxane (17 mL) was thoroughly purged with argon for 20 min. To the stirred solution, water (20 drops) and O,O-diethyl dithiophosphoric acid (2.20 mL, 14.0 mmol) were added dropwise. The reaction mixture was heated to 80° C. and stirred for 65 h. Over time, precipitate was formed. The reaction mixture was allowed to cool to room temperature and diluted with ethyl acetate (100 mL). Then, a saturated solution of Na₂CO₃ (250 mL) and water (100 mL) were added. The reaction mixture was vigorously stirred for 1 h before the organic layer was separated, dried (Na₂SO₄), filtered and concentrated under reduced pressure. The resulting crude product was stirred in dichloromethane to remove small amounts of remaining starting material. The product was filtered to give (1S,5R)-3-(5-chloro-3-pyridyl)-8-(2,2,2-trifluoroethyl)-8-azabicyclo[3.2.1]octane-3-carbothioamide as an off-white powder (m.p. 223-224° C.).

UPLC MS (method H): RT 1.31 min. m/z 364 [M+H]⁺.

¹H NMR (DMSO-d₆, TMS) δ/ppm: 1.65-1.80 (m, 2H), 1.87-2.00 (m, 2H), 2.06 (dd, 2H), 2.45-2.55 (m, 4H), 3.02 (dd, 2H), 3.25 (br s, 2H), 3.50 (dd, 2H), 7.81 (t, 1H), 8.45 (d, 1H), 8.55 (d, 1H), 9.25 (br s, 1H), 9.88 (br s, 1H).

### Example P6

### Preparation of (1 S,5R)-3-(5-bromo-3-pyridyl)-8-(2-chloroallyl)-8-azabicyclo[3.2.1]octane-3-carbothioamide (Compound E.006)

### Step 1: Preparation of (1R,5S)-3-(5-bromo-3-pyridyl)-8-azabicyclo[3.2.1]octane-3-carbonitrile (TFA-salt)

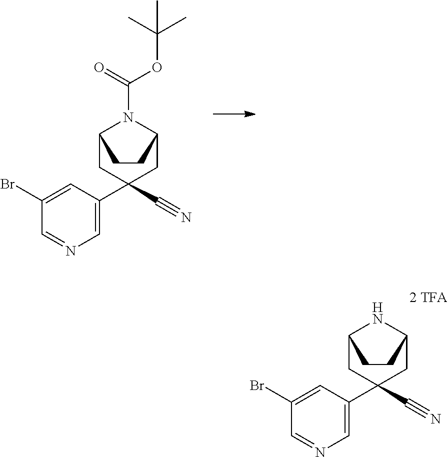 [SMILES: CC(C)(C)OC(=O)N1[C@@H]2CC[C@H]1C[C@](C#N)(c1cncc(Br)c1)C2 ; N#C[C@@]1(c2cncc(Br)c2)C[C@H]2CC[C@@H](C1)N2]

2,2,2-trifluoroacetic acid (TFA) (5.92 mL, 76.5 mmol) was slowly added to a solution of (1S,3S,5R)-3-(5-bromo-pyridin-3-yl)-3-cyano-8-aza-bicyclo[3.2.1]octane-8-carboxylic acid tert-butyl ester (3.00 g, 7.65 mmol) in dichloromethane (38 mL) at 20° C. After completion of the addition, stirring was continued for 8 h. The reaction mixture was concentrated and the residue treated with diethyl ether (20 mL) to form the TFA-salt as a white precipitate. Filtration and drying under vacuum at 40° C. for several hours furnished (1 S,5R)-3-(5-bromo-3-pyridyl)-8-azabicyclo[3.2.1]octane-3-carbonitrile bis-TFA salt as an off-white powder (m.p. 176-178° C.).

¹H NMR (CDCl₃, TMS) δ/ppm: 1.81-2.06 (m, 4H), 2.16-2.26 (m, 2H), 2.28-2.37 (m, 2H), 2.42-2.53 (m, 2H), 3.77 (dd, 2H), 8.00 (t, 1H), 8.63 (d, 1H), 8.73 (d, 1H).

### Step 2: Preparation of (1S,5R)-3-(5-bromo-3-pyridyl)-8-(2-chloroallyl)-8-azabicyclo[3.2.1]octane-3-carbonitrile

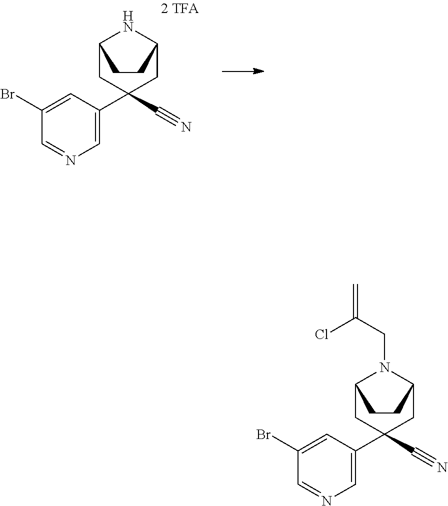 [SMILES: N#C[C@@]1(c2cncc(Br)c2)C[C@H]2CC[C@@H](C1)N2 ; C=C(Cl)CN1[C@@H]2CC[C@H]1C[C@](C#N)(c1cncc(Br)c1)C2]

To a suspension of (1R,5S)-3-(5-bromo-3-pyridyl)-8-azabicyclo[3.2.1]-octane-3-carbonitrile bis-TFA salt (0.700 g, 1.35 mmol) in DMF (6.7 mL) was added dropwise iPr₂NEt (0.95 mL, 5.39 mmol) at room temperature under argon atmosphere. Then, 2,3-dichloro-1-propene (0.200 mL, 2.15 mmol) was added dropwise. The reaction mixture was stirred for 18 h, before heating to 40° C. and addition of a catalytic amount of NaI to drive the reaction to completion within additional 18 h. The reaction mixture was allowed to cool to room temperature and poured into cold water. The resulting mixture was extracted with ethyl acetate (2×), the organic layer was separated and washed with water and brine. After drying (Na₂SO₄), the organic layer was filtered and concentrated in vacuum. The crude product was purified by flash chromatography (SiO₂, 0 to 30% ethyl acetate/cyclohexane) to yield (1S,5R)-3-(5-bromo-3-pyridyl)-8-(2-chloroallyl)-8-azabicyclo[3.2.1]octane-3-carbonitrile as an off-white powder.

UPLC MS (method G): RT 0.64 min. m/z 366 [M+H]⁺.

¹H NMR (CDCl₃, TMS) δ/ppm: 2.08-2.16 (m, 2H), 2.28-2.32 (m, 4H), 2.32-2.41 (m, 2H), 3.15 (s, 2H), 3.42 (br s, 2H), 5.33 (s, 1H), 5.46 (s, 1H), 7.95 (t, 1H), 8.62 (d, 1H), 8.71 (d, 1H).

### Step 3: Preparation of (1S,5R)-3-(5-bromo-3-pyridyl)-8-(2-chloroallyl)-8-azabicyclo[3.2.1]octane-3-carbothioamide (Compound E.006)

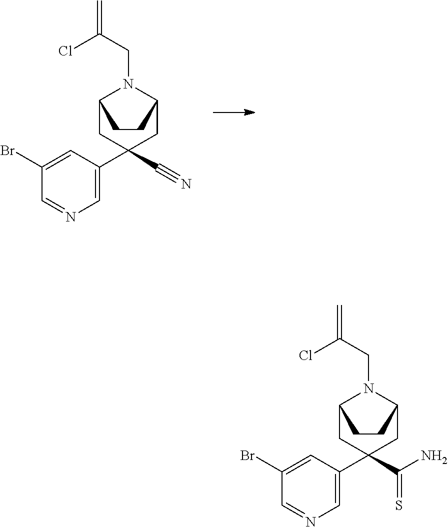 [SMILES: C=C(Cl)CN1[C@@H]2CC[C@H]1C[C@](C#N)(c1cncc(Br)c1)C2 ; C=C(Cl)CN1[C@@H]2CC[C@H]1C[C@](C(N)=S)(c1cncc(Br)c1)C2]

A vial charged with a solution of (1 S,5R)-3-(5-bromo-3-pyridyl)-8-(2-chloroallyl)-8-azabicyclo[3.2.1]octane-3-carbonitrile (1.50 g, 4.50 mmol) in 1,4-dioxane (3.3 mL) was thoroughly purged with argon before sealing. To this stirred solution, water (15 μL, 0.82 mmol) and O,O-diethyl dithiophosphoric acid (0.42 mL, 2.45 mmol) were added dropwise. The reaction mixture was heated to 80° C. and stirred overnight. The reaction mixture was allowed to cool to room temperature and diluted with water and a saturated aqueous solution of Na₂CO₃ which led to the formation of a precipitate. The resulting suspension was vigorously stirred for 2 h before filtering off the solid and drying. Subsequently, the solid was triturated in dichloromethane to remove remaining impurities. Filtration and drying in vacuo furnished (1S,5R)-3-(5-bromo-3-pyridyl)-8-(2-chloroallyl)-8-azabicyclo[3.2.1]octane-3-carbothioamide an off-white powder (m.p. 146-153° C.).

UPLC MS (method G): RT 0.63 min. m/z 399 [M+H]⁺.

¹H NMR (DMSO-d₆, TMS) δ/ppm: 1.65-1.80 (m, 2H), 1.87-2.00 (m, 2H), 2.0-2.10 (d, 2H), 3.05 (br s, 12H), 3.52 (dd, 2H), 5.28 (s, 1H), 5.55 (s, 1H), 7.95 (br s, 1H), 8.52 (d, 1H), 8.58 (d, 1H), 9.25 (br s, 1H), 9.85 (br s, 1H).

### Example P7

### Preparation of (1S,5R)-3-(5-bromo-3-pyridyl)-8-azabicyclo[3.2.1]oct-6-ene-3-carbothioamide (Compound E.015)

### Step 1: Preparation of (1S,5R)-3-cyano-8-aza-bicyclo[3.2.1]oct-6-ene-8-carboxylic acid tert-butyl ester

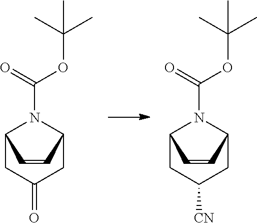 [SMILES: CC(C)(C)OC(=O)N1[C@@H]2C=C[C@H]1CC(=O)C2 ; CC(C)(C)OC(=O)N1[C@@H]2C=C[C@H]1C[C@H](C#N)C2]

Potassium tert-butoxide (1.12 g, 9.52 mmol) was suspended at 0° C. in 1,2-dimethoxyethane (DME) (3.0 mL) under argon atmosphere. Subsequently, within 30 min, a solution of tosylmethyl isocyanide (1.11 g, 5.71 mmol) in DME (3.0 mL) was added dropwise while keeping the temperature below 5° C. The reaction mixture turned immediately brown and was stirred for an additional 1 h at 0° C. After dropwise addition of isopropanol (0.58 mL, 7.61 mmol) at 0° C., stirring was continued at this temperature for 30 min. To this mixture, a solution of (1 S,5R)-3-oxo-8-azabicyclo[3.2.1]oct-6-ene-8-carboxylic acid tert-butyl ester (0.85 g, 3.81 mmol) (prepared according to Hodgson et al., Org. Lett. 2010, 12, 2834) in DME (2.0 mL) was added dropwise within 30 min while maintaining the reaction temperature below 5° C. After completion of the addition, stirring was continued for 1 h at 0° C. and then allowed to warm to room temperature overnight. The reaction mixture was filtered over Celite and the residue was repeatedly washed with ethyl acetate. The organic layers were combined and concentrated under reduced pressure to give the crude product. The crude material was dissolved in ethyl acetate and the resultant organic solution washed with water and brine, dried (MgSO₄), filtered and concentrated. The residue was purified by flash chromatography (SiO₂, 1-28% ethyl acetate/cyclohexane) (1S,5R)-3-cyano-8-aza-bicyclo[3.2.1]oct-6-ene-8-carboxylic acid tert-butyl ester as light orange oil.

¹H NMR (CDCl₃, TMS) δ/ppm: 1.48 (s, 9H), 1.70-1.80 (br m, 2H), 1.80-1.97 (br m, 1H), 1.97-2.10 (br m, 1H), 2.90-3.05 (m, 1H), 4.50-4.67 (br s, 2H), 6.05-6.15 (br m, 2H).

Another ¹H NMR-signal could be detected for a second rotamer: 6.28-6.35 (br m, 2H).

### Step 2: Preparation of (1 S,5R)-3-(5-bromo-pyridin-3-yl)-3-cyano-8-aza-bicyclo[3.2.1]oct-6-ene-8-carboxylic acid tert-butyl ester

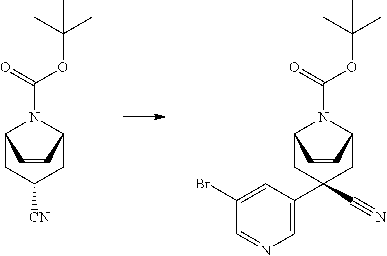 [SMILES: CC(C)(C)OC(=O)N1[C@@H]2C=C[C@H]1C[C@H](C#N)C2 ; CC(C)(C)OC(=O)N1[C@@H]2C=C[C@H]1C[C@](C#N)(c1cncc(Br)c1)C2]

To a stirred solution of (1 S,5R)-3-cyano-8-aza-bicyclo[3.2.1]oct-6-ene-8-carboxylic acid tert-butyl ester (7.50 g, 32.0 mmol) and 3-bromo-5-fluoro-pyridine (5.91 g, 33.6 mmol) in tetrahydrofuran (80 mL) (THF) was added dropwise lithium bis(trimethylsilyl)amide (35.2 mL, 1M in THF) at −30° C. within 20 min under argon atmosphere. The reaction mixture turned immediately brown and stirring was continued at −30° C. for an additional 30 min. The cooling bath was removed and the reaction mixture was allowed to warm to room temperature. The reaction mixture was stirred for an additional 2 h and then poured into cold water and extracted with ethyl acetate (3×). The combined extracts were washed with brine, dried (MgSO₄) and evaporated under reduced pressure to give a light brown oil. Flash chromatography (SiO₂, ethyl acetate/cyclohexane) of the crude product gave (1 S,5R)-3-(5-bromo-pyridin-3-yl)-3-cyano-8-aza-bicyclo[3.2.1]oct-6-ene-8-carboxylic acid tert-butyl ester as light yellow oil.

¹H NMR (CDCl₃, TMS) δ/ppm: 1.55 (s, 9H), 2.12-2.25 (br m, 3H), 2.35-2.47 (br m, 1H), 4.67 (br s, 1H), 4.80 (br s, 1H), 4.80 (br s, 1H), 6.35-6.48 (br m, 2H), 7.90 (t, 1H), 8.65 (dd, 2H).

### Step 3: Preparation of (1S,5R)-3-(5-bromo-3-pyridyl)-8-azabicyclo[3.2.1]oct-6-ene-3-carbonitrile (TFA salt)

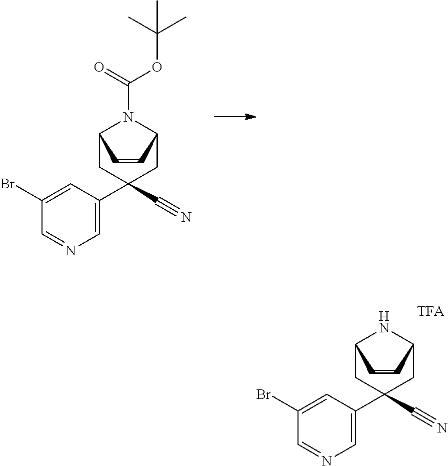 [SMILES: CC(C)(C)OC(=O)N1[C@@H]2C=C[C@H]1C[C@](C#N)(c1cncc(Br)c1)C2 ; N#C[C@@]1(c2cncc(Br)c2)C[C@H]2C=C[C@@H](C1)N2]

To a solution of (1S,5R)-3-(5-bromo-pyridin-3-yl)-3-cyano-8-aza-bicyclo[3.2.1]oct-6-ene-8-carboxylic acid tert-butyl ester (8.50 g, 17.0 mmol) in dichlormethane (87 mL) at 20° C. was slowly added 2,2,2-trifluoroacetic acid (TFA) (13.0 mL, 170 mmol). The reaction mixture was stirred at room temperature overnight. After completion of the reaction ethyl acetate was added. The mixture was washed with aqueous NaHCO₃ (2×) and Na₂CO₃ (2×) solution. The organic layer was separated, dried (Na₂SO₄), filtered and concentrated. The crude material was triturated with diethyl ether to give (1 S,5R)-3-(5-bromo-3-pyridyl)-8-azabicyclo[3.2.1]oct-6-ene-3-carbonitrile as TFA-salt.

¹H NMR (400 MHz, DMSO-d₆) δ/ppm: 2.05-2.14 (m, 2H), 2.15-2.23 (m, 2H), 2.81 (br s, 1H), 3.32 (s, 1H), 3.93 (br s, 2H), 6.28-6.39 (m, 2H), 8.20 (t, 1H), 8.70 (d, 1H), 8.76 (d, 1H).

### Step 4: Preparation of (1S,5R)-3-(5-bromo-3-pyridyl)-8-azabicyclo[3.2.1]oct-6-ene-3-carbothioamide (Compound E.015)

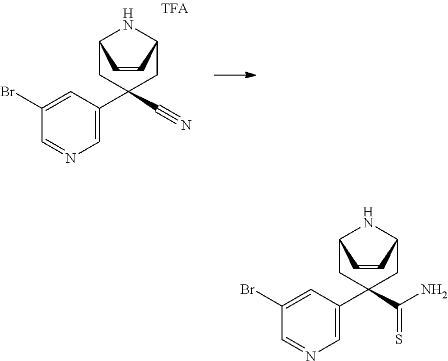 [SMILES: N#C[C@@]1(c2cncc(Br)c2)C[C@H]2C=C[C@@H](C1)N2 ; NC(=S)[C@@]1(c2cncc(Br)c2)C[C@H]2C=C[C@@H](C1)N2]

A round-bottom flask equipped with a magnetic stirring bar was charged with (1S,5R)-3-(5-bromo-3-pyridyl)-8-azabicyclo[3.2.1]oct-6-ene-3-carbonitrile TFA salt (0.50 g, 1.72 mmol), 1,4-dioxane (6.9 mL), and water (0.68 mL, 37.9 mmol) at room temperature. The reaction mixture was purged with argon for 15 min before O,O-diethyl dithiophosphoric acid (0.96 mL, 5.17 mmol) were added dropwise under stirring. The reaction mixture was heated to 80° C. and stirred for an additional 19 h. Subsequently, the reaction mixture was allowed to cool to room temperature and poured into a saturated solution of Na₂CO₃ (50 mL). The reaction mixture was extracted with ethyl acetate (3×), the combined organic layers were dried (Na₂SO₄) and concentrated in vacuo. The residue was triturated in dichloromethane (10 mL) and the precipitate was filtrated to furnish (1 S,5R)-3-(5-bromo-3-pyridyl)-8-azabicyclo[3.2.1]oct-6-ene-3-carbothioamide as light yellow powder.

UPLC MS (method G): RT 0.31 min. m/z 324 [M+H]⁺.

¹H NMR (400 MHz, DMSO-d₆) δ/ppm: 2.00 (dd, 2H), 3.42 (dd, 2H), 3.74 (br s, 2H), 6.02 (s, 2H), 7.92 (t, 1H), 8.54 (d, 1H), 8.58 (d, 1H), 8.99 (br s, 1H), 9.49 (br s, 1H).

### Example P8

### Preparation of (1R,5S)-3-(5-bromo-3-pyridyl)-8-(2-chloroallyl)-8-azabicyclo[3.2.1]oct-6-ene-3-carbothioamide (Compound E.013)

### Step 1: Preparation of (1S,5R)-3-(5-bromo-3-pyridyl)-8-(2-chloroallyl)-8-azabicyclo[3.2.1]oct-6-ene-3-carbonitrile

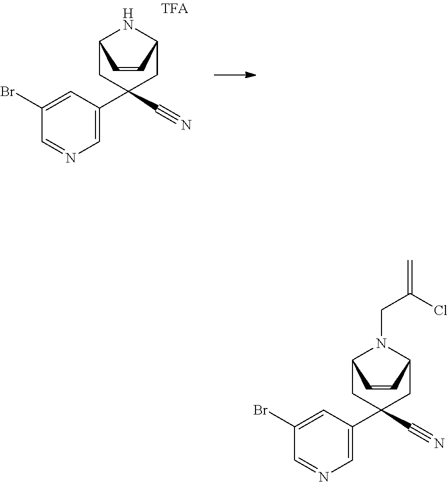 [SMILES: N#C[C@@]1(c2cncc(Br)c2)C[C@H]2C=C[C@@H](C1)N2 ; C=C(Cl)CN1[C@@H]2C=C[C@H]1C[C@](C#N)(c1cncc(Br)c1)C2]

To a suspension of (1 S,5R)-3-(5-bromo-3-pyridyl)-8-azabicyclo[3.2.1]oct-6-ene-3-carbonitrile TFA salt (1.00 g, 3.45 mmol) in DMF (17 mL) was added dropwise iPr₂NEt (1.20 mL, 6.89 mmol) at room temperature under argon atmosphere. Subsequently, 2,3-dichloro-1-propene (0.490 mL, 5.17 mmol) was added dropwise and after completion of the addition stirring was continued for 19 h at room temperature. The reaction mixture was taken up in water (20 mL) and extracted with ethyl acetate (3×). The combined organic layers were washed with brine, dried (Na₂SO₄) and concentrated under reduced pressure. The crude material was purified by flash chromatography (SiO₂, ethyl acetate/heptane) to furnish (1R,5S)-3-(5-bromo-3-pyridyl)-8-(2-chloroallyl)-8-azabicyclo[3.2.1]oct-6-ene-3-carbonitrile as an off-white powder.

UPLC MS (method G): RT 0.76 min. m/z 364 [M+H]⁺.

¹H NMR (CDCl₃, TMS) δ/ppm: 2.20-2.40 (m, 4H), 3.19 (s, 2H), 3.81 (br s, 2H), 5.38 (s, 1H), 5.46 (s, 1H), 6.31 (s, 2H), 8.02 (t, 1H), 8.65 (d, 1H), 8.78 (d, 1H).

### Step 2: Preparation of (1R,5S)-3-(5-bromo-3-pyridyl)-8-(2-chloroallyl)-8-azabicyclo[3.2.1]oct-6-ene-3-carbothioamide (Compound E.013)

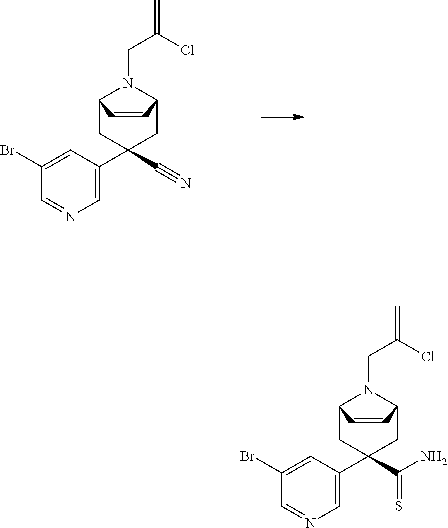 [SMILES: C=C(Cl)CN1[C@@H]2C=C[C@H]1C[C@](C#N)(c1cncc(Br)c1)C2 ; C=C(Cl)CN1[C@@H]2C=C[C@H]1C[C@](C(N)=S)(c1cncc(Br)c1)C2]

A round-bottom flask equipped with a magnetic stirring bar was charged with (1R,5S)-3-(5-bromo-3-pyridyl)-8-(2-chloroallyl)-8-azabicyclo[3.2.1]oct-6-ene-3-carbonitrile (0.81 g, 2.21 mmol) and water (0.40 mL, 2.21 mmol) in 1,4-dioxane (8.8 mL) at room temperature. The reaction mixture was purged with argon for 15 min. before O,O-diethyl dithiophosphoric acid (1.23 mL, 6.62 mmol) were added dropwise under stirring. The reaction mixture was heated to 80° C. and stirred for an additional 28 h. Subsequently, the reaction mixture was allowed to cool to room temperature and poured into a saturated solution of Na₂CO₃ (30 mL). After stirring for 15 min the precipitate was filtered off and dried to give (1R,5S)-3-(5-bromo-3-pyridyl)-8-(2-chloroallyl)-8-azabicyclo[3.2.1]oct-6-ene-3-carbothioamide as an off-white powder.

UPLC MS (method G): RT 0.58 min. m/z 398 [M+H]⁺.

¹H NMR (400 MHz, DMSO-d₆) δ/ppm: 2.06 (dd, 2H), 3.01 (br s, 2H), 3.51-3.62 (m, 4H), 5.30 (br s, 1H), 5.53 (br s, 1H), 5.88 (br s, 2H), 7.91 (t, 1H), 8.52 (d, 1H), 8.56 (d, 1H), 9.02 (br s, 1H), 9.45 (br s, 1H).

### Example P9

### Preparation of (1 S,5R)-8-(2-chloroallyl)-3-(5-cyano-3-pyridyl)-8-azabicyclo[3.2.1]oct-6-ene-3-carbothioamide (Compound E.012)

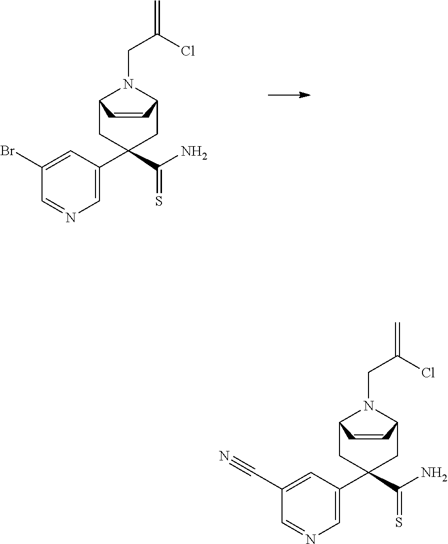 [SMILES: C=C(Cl)CN1[C@@H]2C=C[C@H]1C[C@](C(N)=S)(c1cncc(Br)c1)C2 ; C=C(Cl)CN1[C@@H]2C=C[C@H]1C[C@](C(N)=S)(c1cncc(C#N)c1)C2]

A mixture of (1 S,5R)-3-(5-bromo-3-pyridyl)-8-(2-chloroallyl)-8-azabicyclo[3.2.1]oct-6-ene-3-carbothioamide (650 mg, 1.63 mmol), zinc powder (12.8 mg, 0.20 mmol), Zn(CN)₂ (117 mg, 0.98 mmol), and 1,1′-bis(diphenylphosphino)ferrocene (37.3 mg, 0.070 mmol) were suspended in N,N-dimethyl acetamide (6.0 mL) at room temperature under argon atmosphere. After continuous purging with argon for 20 min, Pd₂(dba)₃ (30.1 mg, 0.033 mmol) was added to give a light yellow suspension. The reaction mixture was heated to 135° C. and stirred for 1.5 h. The reaction mixture was allowed to cool down to room temperature and quenched with aqueous ammonia (20 mL of a 2M solution), filtered through Celite and extracted with ethyl acetate (3×). The combined organic layers were washed with brine, dried (Na₂SO₄), filtered and evaporated in vacuo. The crude product was purified by flash chromatography (SiO₂, ethyl acetate/heptane). The obtained material was triturated in dichloromethane and the obtained solid subjected to another purification by flash chromatography to furnish (1 S,5R)-8-(2-chloroallyl)-3-(5-cyano-3-pyridyl)-8-azabicyclo[3.2.1]oct-6-ene-3-carbothioamide as an off-white powder.

UPLC MS (method G): RT 0.39 min. m/z 345 [M+H]⁺.

¹H NMR (400 MHz, DMSO-d₆) δ/ppm: 2.02-2.15 (m, 2H), 3.02 (br s, 2H), 3.51-3.64 (m, 4H), 5.29 (br s, 1H), 5.51 (br s, 1H), 5.88 (br s, 2H), 8.15 (t, 1H), 8.81 (d, 1H), 8.86 (d, 1H), 9.00 (br s, 1H), 9.50 (br s, 1H).

### Example P10

### Preparation of (1R,5S)-3-(5-chloro-3-pyridyl)-8-(2,2-difluoroethyl)-8-azabicyclo[3.2.1]oct-6-ene-3-carbothioamide (Compound E.001)

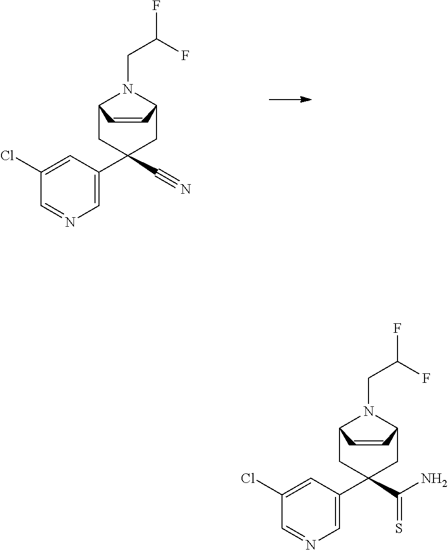 [SMILES: N#C[C@@]1(c2cncc(Cl)c2)C[C@H]2C=C[C@@H](C1)N2CC(F)F ; NC(=S)[C@@]1(c2cncc(Cl)c2)C[C@H]2C=C[C@@H](C1)N2CC(F)F]

A round-bottom flask equipped with a magnetic stirring bar was charged with (1R,5S)-3-(5-chloro-3-pyridyl)-8-(2,2-difluoroethyl)-8-azabicyclo[3.2.1]oct-6-ene-3-carbonitrile (prepared according to WO 96/37494) (0.30 g, 0.97 mmol) and water (two drops) in 1,4-dioxane (3.9 mL) at room temperature. The reaction mixture was purged with argon for 10 min before O,O-diethyl dithiophosphoric acid (0.50 mL, 2.91 mmol) was added dropwise under stirring. The reaction mixture was heated to 80° C. and stirred overnight with concomitant formation of a precipitate. Subsequently, the reaction mixture was allowed to cool to room temperature and poured into a saturated solution of Na₂CO₃ and water. After extraction with ethyl acetate (3×), the combined organic layers were dried (Na₂SO₄), filtered and concentrated under reduced pressure. After trituration of the residual with a small amount of dichloromethane (1R,5S)-3-(5-chloro-3-pyridyl)-8-(2,2-difluoroethyl)-8-azabicyclo[3.2.1]oct-6-ene-3-carbothioamide was obtained as an off-white powder.

UPLC MS (method G): RT 0.55 min. m/z 344 [M+H]⁺.

¹H NMR (400 MHz, DMSO-d₆) δ/ppm: 1.65-1.80 (m, 2H), 1.88-2.00 (m, 2H), 2.03-2.12 (m, 2H), 2.96-3.10 (dd, 2H), 3.26 (br s, 2H), 3.45-3.58 (dd, 2H), 7.81 (t, 1H), 8.47 (d, 1H), 8.55 (d, 1H), 9.25 (br s, 1H), 9.87 (br s, 1H).

### Example P11

### Preparation of (1R,5S)-3-(5-bromo-3-pyridyl)-8-(4,4,4-trifluorobutyl)-8-azabicyclo[3.2.1]oct-6-ene-3-carbothioamide (Compound E.008)

### Step 1: Preparation of (1R,5S)-3-(5-bromo-3-pyridyl)-8-(4,4,4-trifluorobutyl)-8-azabicyclo[3.2.1]oct-6-ene-3-carbonitrile

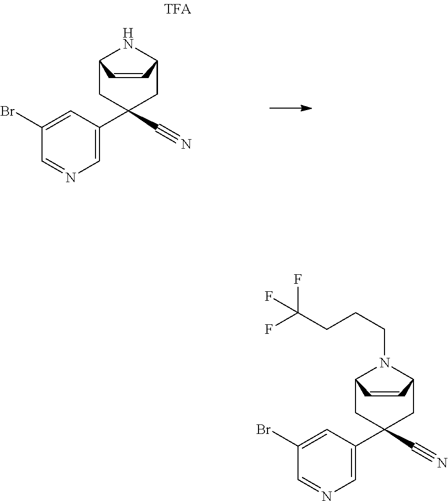 [SMILES: N#C[C@@]1(c2cncc(Br)c2)C[C@H]2C=C[C@@H](C1)N2 ; N#C[C@@]1(c2cncc(Br)c2)C[C@H]2C=C[C@@H](C1)N2CCCC(F)(F)F]

To a suspension of (1 S,5R)-3-(5-bromo-3-pyridyl)-8-azabicyclo[3.2.1]oct-6-ene-3-carbonitrile TFA salt (0.45 g, 1.11 mmol) in acetonitrile (5.0 mL) was added dropwise iPr₂NEt (0.76 mL, 4.45 mmol) at room temperature under argon atmosphere. Stirring of the suspension for 10 min resulted in a clear yellow solution. Subsequently, 4-bromo-1,1,1-trifluoro-butane (0.15 mL, 1.23 mmol) was added dropwise and the reaction mixture was stirred for 72 h at room temperature. After dilution with ethyl acetate, the reaction mixture and washed with aqueous NaHCO₃. The organic layer was separated, dried (Na₂SO₄) and concentrated in vacuo. The crude material was purified by flash chromatography (SiO₂, 0-40% MeOH/dichloromethane) to obtain (1R,5S)-3-(5-bromo-3-pyridyl)-8-(4,4,4-trifluorobutyl)-8-azabicyclo[3.2.1]oct-6-ene-3-carbonitrile as a gum.

UPLC MS (method H): RT 0.68 min. m/z 400 [M+H]⁺.

¹H NMR (CDCl₃, TMS) δ/ppm: 1.65-1.75 (m, 2H), 2.12-2.28 (m, 2H), 2.25-2.28 (m, 4H), 2.40 (t, 2H), 3.75 (br s, 2H), 6.28 (s, 2H), 7.89 (t, 1H), 8.61 (d, 1H), 8.71 (d, 1H).

### Step 2: Preparation of (1R,5S)-3-(5-bromo-3-pyridyl)-8-(4,4,4-trifluorobutyl)-8-azabicyclo[3.2.1]oct-6-ene-3-carbothioamide (Compound E.008)

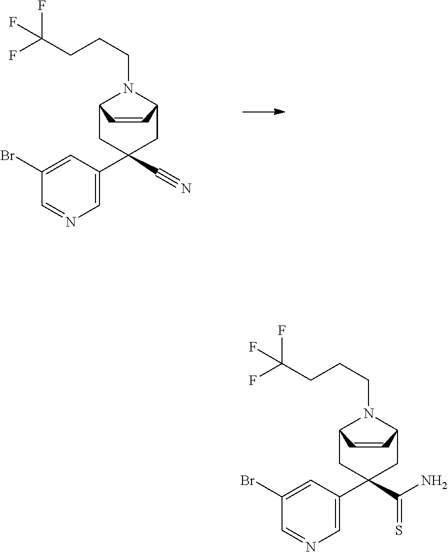 [SMILES: N#C[C@@]1(c2cncc(Br)c2)C[C@H]2C=C[C@@H](C1)N2CCCC(F)(F)F ; NC(=S)[C@@]1(c2cncc(Br)c2)C[C@H]2C=C[C@@H](C1)N2CCCC(F)(F)F]

A round-bottom flask equipped with a magnetic stirring bar was charged with (1R,5S)-3-(5-bromo-3-pyridyl)-8-(4,4,4-trifluorobutyl)-8-azabicyclo[3.2.1]oct-6-ene-3-carbonitrile (320 mg, 0.80 mmol) and water (two drops) in 1,4-dioxane (2.1 mL) at room temperature. The reaction mixture was purged with argon for 10 min before O,O-diethyl dithiophosphoric acid (0.30 mL, 1.60 mmol) was added dropwise under stirring. The reaction mixture was heated to 80° C. and stirred 18 h. Subsequently, the reaction mixture was allowed to cool to room temperature, diluted with ethyl acetate and poured into a saturated solution of Na₂CO₃ (20 mL). After vigorous stirring of this mixture for 1 h, the organic layer was separated, dried (Na₂SO₄), filtered and concentrated under reduced pressure. After trituration of the residual with a small amount of dichloromethane (1R,5S)-3-(5-bromo-3-pyridyl)-8-(4,4,4-trifluorobutyl)-8-azabicyclo[3.2.1]oct-6-ene-3-carbothioamide was obtained as an off-white powder (m.p. 200-202° C.).

UPLC MS (method H): RT 0.76 min. m/z 434 [M+H]⁺.

¹H NMR (400 MHz, DMSO-d₆) δ/ppm: 1.47-1.50 (m, 2H), 1.95-2.08 (d, 2H), 2.14-2.35 (m, 4H), 3.40-3.55 (m, 2H), 3.51 (br s, 2H), 5.85 (s, 2H), 7.89 (t, 1H), 8.50-8.58 (d (×2), 2H), 8.98 (br s, 1H), 9.45 (br s, 1H).

### Example P12

### Preparation of methyl (1S,5R)-3-(5-chloro-3-pyridyl)-8-(2,2,2-trifluoroethyl)-8-azabicyclo[3.2.1]octane-3-carboximidothioate (Compound E.014)

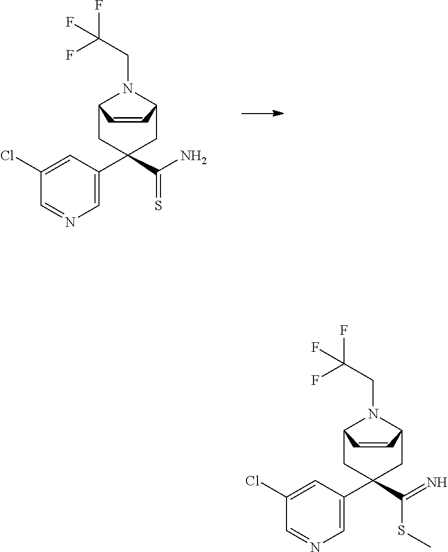 [SMILES: NC(=S)[C@@]1(c2cncc(Cl)c2)C[C@H]2C=C[C@@H](C1)N2CC(F)(F)F ; CSC(=N)[C@@]1(c2cncc(Cl)c2)C[C@H]2C=C[C@@H](C1)N2CC(F)(F)F]

To a solution of (1 S,5R)-3-(5-chloro-3-pyridyl)-8-(2,2,2-trifluoroethyl)-8-azabicyclo[3.2.1]octane-3-carbothioamide (509 mg, 1.40 mmol) in DMF (3.5 mL) at 0° C. was added sodium hydride (61.6 mg, 1.54 mmol, 60 wt % in mineral oil) and stirring at this temperature was continued for 20 min. After addition of iodomethane (87.2 μL, 1.40 mmol), stirring at 0° C. was continued for an additional 1 h. The reaction mixture was slowly poured into aqueous NaHCO₃, the aqueous layer was extracted with ethyl acetate (3×) and the combined organic layers were washed with water and brine. After drying (Na₂SO₄) of the organic layer, filtration and concentration in vacuo, the residual was subjected to flash chromatography (SiO₂, MeOH/dichloromethane) to furnish (1 S,5R)-3-(5-chloro-3-pyridyl)-8-(2,2,2-trifluoroethyl)-8-azabicyclo[3.2.1]octane-3-carboximidothioate as an orange gum.

UPLC MS (method H): RT 1.50 min. m/z 378 [M+H]+.

¹H NMR (400 MHz, CDCl₃) δ/ppm: 1.72-1.83 (m, 2H), 1.93-2.03 (m, 2H), 2.12 (s, 3H), 2.24 (d, 2H), 2.84 (q, 2H), 3.21 (d, 2H), 3.37 (br s, 2H), 7.68 (t, 1H), 8.39 (d, 1H), 8.52 (d, 1H), 9.50 (br s, 1H).

### Example P14

### Preparation of (1S,5R)-3-(5-iodo-3-pyridyl)-8-azabicyclo[3.2.1]oct-6-ene-3-carbothioamide (Compound E.034)

### Step 1: Preparation of tert-butyl (1R,5S)-3-cyano-3-(5-iodo-3-pyridyl)-8-azabicyclo[3.2.1]oct-6-ene-8-carboxylate

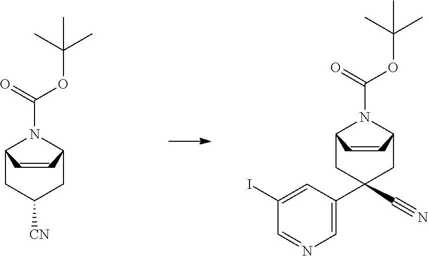 [SMILES: CC(C)(C)OC(=O)N1[C@@H]2C=C[C@H]1C[C@H](C#N)C2 ; CC(C)(C)OC(=O)N1[C@@H]2C=C[C@H]1C[C@](C#N)(c1cncc(I)c1)C2]

Lithium bis(trimethylsilyl)amide (19.2 mL of a 1M solution in THF) was added dropwise to a stirred solution of tert-butyl (1R,5S)-3-hydroxy-8-azabicyclo[3.2.1]oct-6-ene-8-carboxylate (3.0 g, 12.8 mmol) and 3-fluoro-5-iodo-pyridine (3.20 g, 14.1 mmol) in tetrahydrofuran (THF) (100 mL) at −40° C. over 1 h under argon atmosphere. The reaction mixture turned immediately brown. Stirring was continued at −40° C. for 1 h and then allowed to warm to room temperature during 4 h. The reaction mixture was then poured into cold water and extracted with ethyl acetate (3×). The combined extracts were washed with brine, dried (MgSO₄) and evaporated under reduced pressure to give the crude product. Purification by flash chromatography (SiO₂, ethyl acetate/heptane gradient) furnished tert-butyl (1R,5S)-3-cyano-3-(5-iodo-3-pyridyl)-8-azabicyclo[3.2.1]oct-6-ene-8-carboxylate as a white solid.

¹H NMR (400 MHz, CDCl₃, TMS) δ/ppm: 1.52 (s, 9H), 2.10-2.25 (m, 3H), 2.35-2.45 (br m, 1H), 4.65-4.82 (br m, 2H), 6.37-6.45 (br m, 2H), 8.07 (t, 1H), 8.67 (d, 1H), 8.78 (d, 1H).

### Step 2: Preparation of (1S,5R)-3-(5-iodo-3-pyridyl)-8-azabicyclo[3.2.1]oct-6-ene-3-carbonitrile

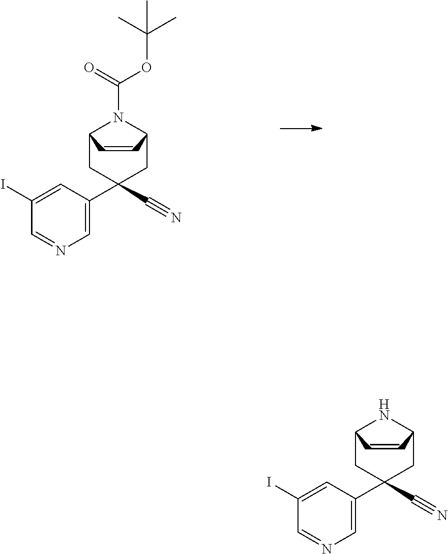 [SMILES: CC(C)(C)OC(=O)N1[C@@H]2C=C[C@H]1C[C@](C#N)(c1cncc(I)c1)C2 ; N#C[C@@]1(c2cncc(I)c2)C[C@H]2C=C[C@@H](C1)N2]

2,2,2-trifluoroacetic acid (TFA) (6.0 mL, 77.5 mmol) was slowly added to a solution of tert-butyl (1R,5S)-3-cyano-3-(5-iodo-3-pyridyl)-8-azabicyclo[3.2.1]oct-6-ene-8-carboxylate (3.39 g, 7.75 mmol) in dichloromethane (14 mL) at 20° C. After completion of the addition, stirring was continued overnight. The reaction mixture was concentrated and the residue treated with ethyl acetate (100 mL). Then a solution of saturated Na₂CO₃ (100 mL) was added slowly. After thorough extraction with ethyl acetate all organic layers were combined, washed with brine, dried over Na₂SO₄ and concentrated under vacuum to furnish (1S,5R)-3-(5-iodo-3-pyridyl)-8-azabicyclo[3.2.1]oct-6-ene-3-carbonitrile.

¹H NMR (400 MHz, CDCl₃, TMS) δ/ppm: 2.19 (dd, 2H), 2.28 (dd, 2H), 4.0-4.10 (m, 2H), 6.45-6.55 (m, 2H), 8.20 (t, 1H), 8.71-8.82 (2d, 2H).

### Step 3: Preparation of (1S,5R)-3-(5-iodo-3-pyridyl)-8-azabicyclo[3.2.1]oct-6-ene-3-carbothioamide (Compound E.034)

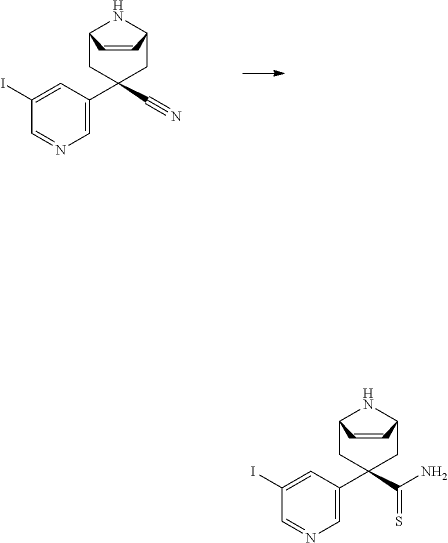 [SMILES: N#C[C@@]1(c2cncc(I)c2)C[C@H]2C=C[C@@H](C1)N2 ; NC(=S)[C@@]1(c2cncc(I)c2)C[C@H]2C=C[C@@H](C1)N2]

A round-bottom flask equipped with a magnetic stirring bar was charged with (1S,5R)-3-(5-iodo-3-pyridyl)-8-azabicyclo[3.2.1]oct-6-ene-3-carbonitrile (2.18 g, 6.46 mmol), 1,4-dioxane (26 mL), and water (2.6 mL, 142.0 mmol) at room temperature. The reaction mixture was purged with argon for 15 min before O,O-diethyl dithiophosphoric acid (3.6 mL, 19.37 mmol) were added dropwise under stirring. The reaction mixture was heated to 80° C. and stirred for an additional 24 h. Subsequently, the reaction mixture was allowed to cool to room temperature and poured into a saturated solution of Na₂CO₃ (50 mL). The reaction mixture was extracted with ethyl acetate (3×), the combined organic layers were dried (Na₂SO₄) and concentrated in vacuo. The residue was triturated with dichloromethane and the precipitation was filtered to furnish (1S,5R)-3-(5-iodo-3-pyridyl)-8-azabicyclo[3.2.1]oct-6-ene-3-carbothioamide as light yellow powder.

¹H NMR (400 MHz, DMSO-d₆, TMS) δ/ppm: 1.95 (dd, 2H), 3.49 (dd, 2H), 3.71 (br s, 2H), 5.99 (s, 2H), 8.05 (t, 1H), 8.54 (d, 1H), 8.61 (d, 1H), 8.95 (br s, 1H), 9.49 (br s, 1H).

### Example P15

### Preparation of (1 S,5R)-8-(2,2-difluoroethyl)-3-(5-iodo-3-pyridyl)-8-azabicyclo[3.2.1]oct-6-ene-3-carbothioamide (Compound E.039)

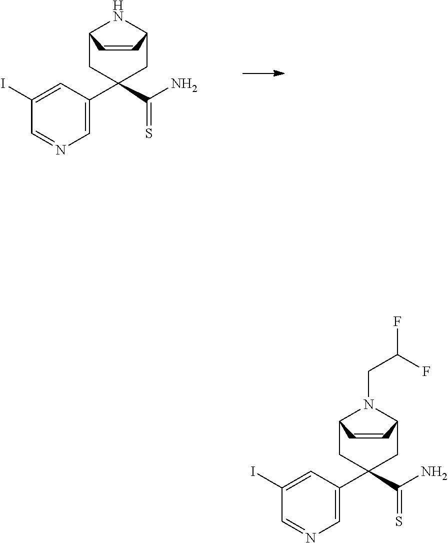 [SMILES: NC(=S)[C@@]1(c2cncc(I)c2)C[C@H]2C=C[C@@H](C1)N2 ; NC(=S)[C@@]1(c2cncc(I)c2)C[C@H]2C=C[C@@H](C1)N2CC(F)F]

To a suspension of (1 S,5R)-3-(5-iodo-3-pyridyl)-8-azabicyclo[3.2.1]oct-6-ene-3-carbothioamide (0.20 g, 0.54 mmol) and K₂CO₃ (0.15 g, 1.08 mmol) in DMF (2.2 mL) was added dropwise 2,2-difluoroethyl trifluoromethanesulfonate (0.23 g, 1.08 mmol) at room temperature under argon atmosphere. The reaction mixture was stirred at room temperature for 18 h. An additional amount of 2,2-difluoroethyl trifluoromethanesulfonate (0.12 g, 0.54 mmol) was added and the reaction mixture was stirred for a further 1 h to drive the reaction to completion. Then the reaction mixture was poured into cold water. The resulting mixture was extracted with ethyl acetate (2×), the organic layer was separated and washed with water and brine. After drying (Na₂SO₄), the organic layer was filtered and concentrated in vacuum. The crude product was stirred in dichloro methane and the resulting precipitation filtered to yield pure (1 S,5R)-8-(2,2-difluoroethyl)-3-(5-iodo-3-pyridyl)-8-azabicyclo[3.2.1]oct-6-ene-3-carbothioamide as a light yellow powder.

¹H NMR (400 MHz, DMSO-d₆, TMS) δ/ppm: 2.01 (dd, 2H), 2.50-2.70 (m, 2H), 3.50 (dd, 2H), 3.62 (br s, 2H), 5.89 (s, 1H), 5.99 (tt, 1H), 8.03 (t, 1H), 8.52 (d, 1H), 8.62 (d, 1H), 8.98 (br s, 1H), 9.50 (br s, 1H).

### Example P16

### Preparation of (1R,5S)-3-(5-iodo-3-pyridyl)-8-(2,2,2-trifluoroethyl)-8-azabicyclo[3.2.1]oct-6-ene-3-carbothioamide (Compound E.060)

### Step 1: Preparation of (1R,5S)-3-(5-iodo-3-pyridyl)-8-(2,2,2-trifluoroethyl)-8-azabicyclo[3.2.1]oct-6-ene-3-carbonitrile

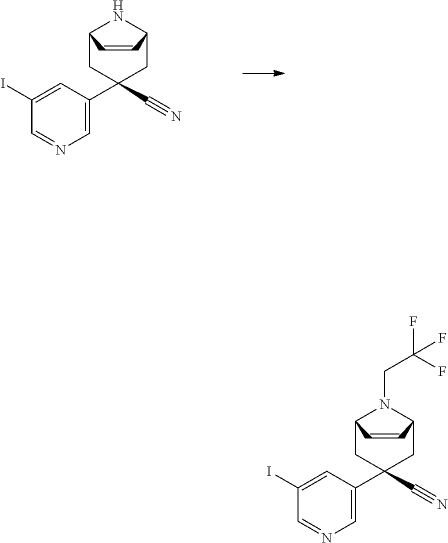 [SMILES: N#C[C@@]1(c2cncc(I)c2)C[C@H]2C=C[C@@H](C1)N2 ; N#C[C@@]1(c2cncc(I)c2)C[C@H]2C=C[C@@H](C1)N2CC(F)(F)F]

To a suspension of (1S,5R)-3-(5-iodo-3-pyridyl)-8-azabicyclo[3.2.1]oct-6-ene-3-carbonitrile (0.80 g, 2.37 mmol) and K₂CO₃ (0.98 g, 7.12 mmol) in THF (9.5 mL) was added dropwise 2,2,2-trifluoroethyl trifluoromethanesulfonate (0.62 g, 2.61 mmol) at room temperature under argon atmosphere. The reaction mixture was heated to 50° C. and stirred for 4.5 h. After completion of the reaction the reaction mixture was poured into cold water. The resulting mixture was extracted with ethyl acetate (3×), the organic layer was separated and washed with water and brine. After drying (Na₂SO₄), the organic layer was filtered and concentrated in vacuum. The crude product was stirred in pentane and the resulting precipitation filtered to yield (1R,5S)-3-(5-iodo-3-pyridyl)-8-(2,2,2-trifluoroethyl)-8-azabicyclo[3.2.1]oct-6-ene-3-carbonitrile as beige powder (m.p. 127-129° C.).

¹H NMR (400 MHz, DMSO-d₆, TMS) δ/ppm: 2.25 (dd, 2H), 2.36 (dd, 2H), 2.90 (q, 2H), 3.85 (br s, 2H), 6.35 (s, 2H), 8.13 (t, 1H), 8.75 (d, 1H), 8.79 (d, 1H).

### Step 2: Preparation of (1R,5S)-3-(5-iodo-3-pyridyl)-8-(2,2,2-trifluoroethyl)-8-azabicyclo[3.2.1]oct-6-ene-3-carbothioamide (Compound E.060)

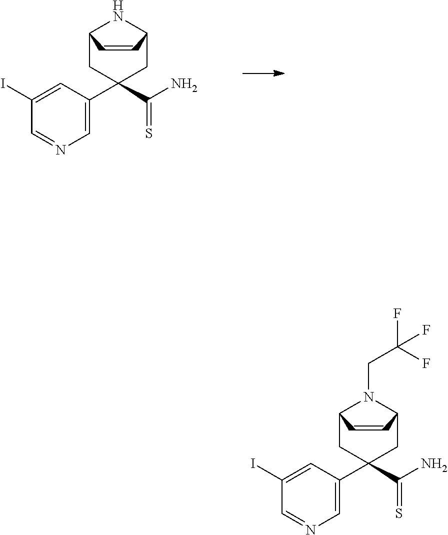 [SMILES: NC(=S)[C@@]1(c2cncc(I)c2)C[C@H]2C=C[C@@H](C1)N2 ; NC(=S)[C@@]1(c2cncc(I)c2)C[C@H]2C=C[C@@H](C1)N2CC(F)(F)F]

A round bottom flask was charged with (1 S,5R)-3-(5-iodo-3-pyridyl)-8-azabicyclo[3.2.1]oct-6-ene-3-carbothioamide (1.50 g, 4.04 mmol) and K₂CO₃ (1.68 g, 12.10 mmol) in DMF (16 mL). To this suspension was added dropwise 2,2,2-trifluoroethyl trifluoromethanesulfonate (1.16 g, 4.85 mmol) at room temperature under argon atmosphere. The reaction mixture was stirred at room temperature for 50 minutes. Then the reaction mixture was poured into cold water. The resulting mixture was extracted with ethyl acetate (3×), the organic layer was separated and washed with water and brine. After drying (Na₂SO₄), the organic layer was filtered and concentrated in vacuum. The crude product was stirred in dichloro methane and the resulting precipitation filtered to yield pure (1R,5S)-3-(5-iodo-3-pyridyl)-8-(2,2,2-trifluoroethyl)-8-azabicyclo[3.2.1]oct-6-ene-3-carbothioamide as light yellow powder.

¹H NMR (400 MHz, DMSO-d₆, TMS) δ/ppm: 2.04 (dd, 2H), 2.93 (q, 2H), 3.52 (dd, 2H), 3.65 (br s, 2H), 5.92 (br s, 1H), 8.05 (t, 1H), 8.53 (d, 1H), 8.63 (d, 1H), 9.0 (br s, 1H), 9.50 (br s, 1H).

### Example P17

### Preparation of (1R,5S)-8-allyl-3-(5-iodo-3-pyridyl)-8-azabicyclo[3.2.1]oct-6-ene-3-carbothioamide (Compound E.063)

### Step 1: Preparation of (1R,5S)-8-allyl-3-(5-iodo-3-pyridyl)-8-azabicyclo[3.2.1]oct-6-ene-3-carbonitrile

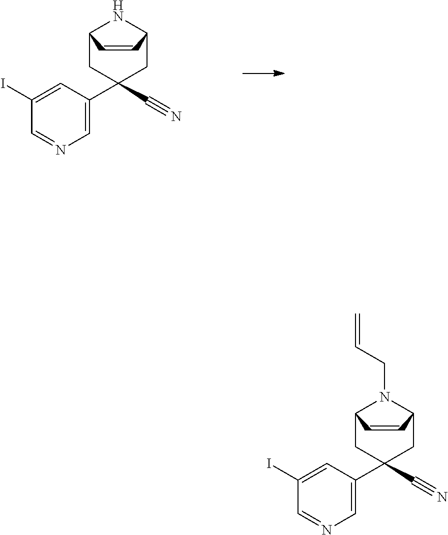 [SMILES: N#C[C@@]1(c2cncc(I)c2)C[C@H]2C=C[C@@H](C1)N2 ; C=CCN1[C@@H]2C=C[C@H]1C[C@](C#N)(c1cncc(I)c1)C2]

To a suspension of (1 S,5R)-3-(5-iodo-3-pyridyl)-8-azabicyclo[3.2.1]oct-6-ene-3-carbonitrile (0.80 g, 2.37 mmol) and K₂CO₃ (0.98 g, 7.12 mmol) in THF (9.5 mL) was added dropwise 3-bromoprop-1-ene (0.32 g, 2.61 mmol) at room temperature under argon atmosphere. The reaction mixture was heated to 50° C. and stirred for 4.5 h. After completion of the reaction the reaction mixture was cooled to room temperature and poured into cold water. The resulting mixture was extracted with ethyl acetate (3×), the organic layer was separated and washed with water and brine. After drying (Na₂SO₄), the organic layer was filtered and concentrated in vacuum. The crude product was stirred in pentane and the resulting precipitation filtered to yield (1R,5S)-8-allyl-3-(5-iodo-3-pyridyl)-8-azabicyclo[3.2.1]oct-6-ene-3-carbonitrile as light orange powder (m.p. 99-100° C.).

¹H NMR (400 MHz, CDCl₃, TMS) δ/ppm: 2.26 (dd, 4H), 3.02 (d, 2H), 3.77 (br s, 2H), 5.08-5.22 (m, 2H), 5.76-5.99 (m, 1H), 6.25 (s, 2H), 8.17 (t, 1H), 8.79 (2d, 2H).

### Step 2: Preparation of (1R,5S)-8-allyl-3-(5-iodo-3-pyridyl)-8-azabicyclo[3.2.1]oct-6-ene-3-carbothioamide (Compound E.063)

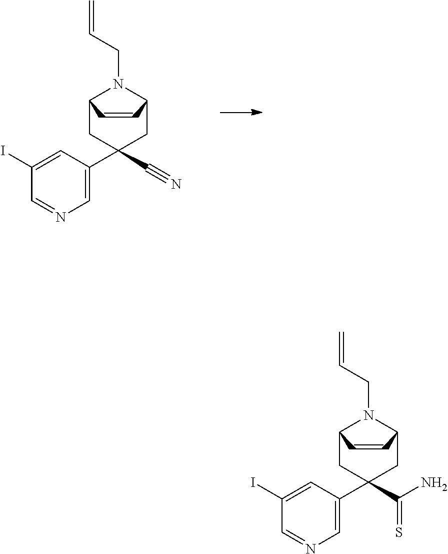 [SMILES: C=CCN1[C@@H]2C=C[C@H]1C[C@](C#N)(c1cncc(I)c1)C2 ; C=CCN1[C@@H]2C=C[C@H]1C[C@](C(N)=S)(c1cncc(I)c1)C2]

A round-bottom flask equipped with a magnetic stirring bar was charged with (1R,5S)-8-allyl-3-(5-iodo-3-pyridyl)-8-azabicyclo[3.2.1]oct-6-ene-3-carbonitrile (0.59 g, 1.55 mmol), 1,4-dioxane (6 mL), and water (0.03 mL, 142.0 mmol) at room temperature. The reaction mixture was purged with argon for 15 min before O,O-diethyl dithiophosphoric acid (0.87 mL, 4.66 mmol) were added dropwise under stirring. The reaction mixture was heated to 80° C. and stirred for additional 18 h. Subsequently, the reaction mixture was allowed to cool to room temperature and poured into a saturated solution of Na₂CO₃ (60 mL). The reaction mixture was extracted with ethyl acetate (3×), the combined organic layers were combined, dried (Na₂SO₄) and concentrated in vacuo. The residue was triturated with dichloromethane and the precipitation was filtered to yield (1R,5S)-8-allyl-3-(5-iodo-3-pyridyl)-8-azabicyclo[3.2.1]oct-6-ene-3-carbothioamide as beige powder (m.p. 186-187° C.).

¹H NMR (400 MHz, DMSO-d₆, TMS) δ/ppm: 1.94-2.08 (m, 2H), 2.85 (d, 2H), 3.43-3.54 (m, 4H), 4.97-5.16 (m, 2H), 5.70-5.89 (m, 3H), 8.03 (t, 1H), 8.56 (d, 1H), 8.73 (d, 1H), 8.93 (br s, 1H), 9.41 (br s, 1H).

### Example P18

### Preparation of (1R,5S)-8-(2,2,2-trifluoroethyl)-3-[5-(2-trimethylsilylethynyl)-3-pyridyl]-8-azabicyclo[3.2.1]oct-6-ene-3-carbothioamide (Compound E.068)

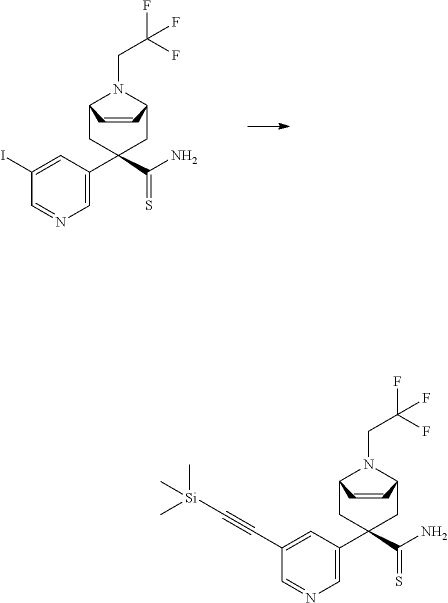 [SMILES: NC(=S)[C@@]1(c2cncc(I)c2)C[C@H]2C=C[C@@H](C1)N2CC(F)(F)F ; C[Si](C)(C)C#Cc1cncc([C@]2(C(N)=S)C[C@H]3C=C[C@@H](C2)N3CC(F)(F)F)c1]

A small round bottom flask was charged with (1R,5S)-3-(5-iodo-3-pyridyl)-8-(2,2,2-trifluoroethyl)-8-azabicyclo[3.2.1]oct-6-ene-3-carbothioamide (0.14 g, 0.30 mmol) and triethylamine (0.06 mL, 0.45 mmol) in DMF (1.2 mL). The reaction mixture was degassed with argon before adding ethynyl(trimethyl)silane (0.06 mL, 0.45 mmol), Cul (0.003 g, 0.015 mmol) and Pd(PPh₃)₂Cl₂ (0.01 g, 0.015 mmol). The reaction mixture was stirred at 55° C. for 3 h. Then the reaction mixture was cooled down to room temperature and filtrated through Celite®. The filtrate was diluted with ethyl acetate and washed with water and brine. After drying (Na₂SO₄), the organic layer was filtered and concentrated in vacuum. The crude product was treated with pentane to afford (1R,5S)-8-(2,2,2-trifluoroethyl)-3-[5-(2-trimethylsilylethynyl)-3-pyridyl]-8-azabicyclo[3.2.1]oct-6-ene-3-carbothioamide as light brown powder (m.p. 179-180° C.).

¹H NMR (400 MHz, DMSO-d₆, TMS) δ/ppm: 0.25 (s, 9H), 2.01-2.11 (m, 2H), 2.96 (q, 2H), 3.50-3.60 (m, 2H), 3.65 (br s, 2H), 5.93 (br s, 2H), 7.76 (t, 1H), 8.47 (d, 1H), 8.55 (d, 1H), 9.01 (br s, 1H), 9.49 (br s, 1H).

### Example P19

### Preparation of (1R,5S)-3-(5-ethynyl-3-pyridyl)-8-(2,2,2-trifluoroethyl)-8-azabicyclo[3.2.1]oct-6-ene-3-carbothioamide (Compound E.070)

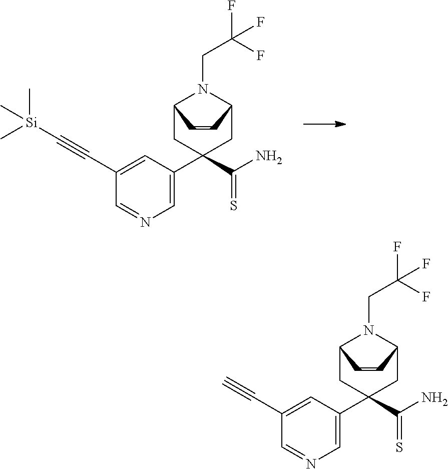 [SMILES: C[Si](C)(C)C#Cc1cncc([C@]2(C(N)=S)C[C@H]3C=C[C@@H](C2)N3CC(F)(F)F)c1 ; C#Cc1cncc([C@]2(C(N)=S)C[C@H]3C=C[C@@H](C2)N3CC(F)(F)F)c1]

A small round bottom flask was charged with (1R,5S)-8-(2,2,2-trifluoroethyl)-3[5-(2-trimethylsilylethynyl)-3-pyridyl]-8-azabicyclo[3.2.1]oct-6-ene-3-carbothioamide (0.20 g, 0.57 mmol) and methanol (4.6 mL) at room temperature. The solution was cooled down to 0° C. Then K₂CO₃ (0.048 g, 0.35 mmol) was added in small portions. The resultant suspension was stirred at 0° C. for 40 minutes. The reaction was allowed to warm up to room temperature and then poured into saturated aqueous NaHCO₃ (10 ml). The mixture was extracted with ethyl acetate (3×). The organic layers were combined, washed with water and brine. After drying over Na₂SO₄, the organic layer was filtered and concentrated in vacuum. The crude product was treated with a mixture of dichloro methane and pentane to afford (1R,5S)-3-(5-ethynyl-3-pyridyl)-8-(2,2,2-trifluoroethyl)-8-azabicyclo[3.2.1]oct-6-ene-3-carbothioamide as beige powder (m.p. 210-213° C.).

¹H NMR (400 MHz, CDCl₃, TMS) δ/ppm: 2.42-2.50 (m, 2H), 2.82 (q, 2H), 3.20 (s, 1H), 3.28-3.35 (m, 2H), 3.72 (br s, 2H), 6.05 (br s, 2H), 7.81 (t, 1H), 8.55 (d, 1H), 8.61 (d, 1H).

Selected signals in DMSO-d₆:

¹H NMR (400 MHz, DMSO-d₆, TMS) δ/ppm: 9.02 (br s, 1H, NH₂), 9.50 (br s, 1H, NH₂).

### Example P20

### Preparation (1R, 5S)-8-(2,2-difluoroethyl)-3-(5-fluoro-3-pyridyl)-8-azabicyclo[3.2.1]oct-6-ene-3-carbothioamide (Compound E.071)

### Step 1: Preparation of tert-butyl (1R,5S)-3-cyano-3-(5-fluoro-3-pyridyl)-8-azabicyclo[3.2.1]oct-6-ene-8-carboxylate

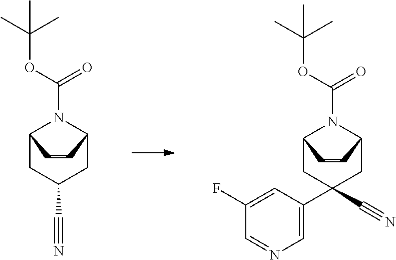 [SMILES: CC(C)(C)OC(=O)N1[C@@H]2C=C[C@H]1C[C@H](C#N)C2 ; CC(C)(C)OC(=O)N1[C@@H]2C=C[C@H]1C[C@](C#N)(c1cncc(F)c1)C2]

tert-Butyl (1R,5S)-3-cyano-8-azabicyclo[3.2.1]oct-6-ene-8-carboxylate (10.0 g, 42.7 mmol) was dissolved in anhydrous tetrahydrofuran (100 mL) in a dry 500 mL three neck flask and 3,5-difluoropyridine (5.15 g, 44.8 mmol) was added to the stirred solution under nitrogen. The reaction mixture was cooled down to −50° C. and lithium bis(trimethylsilyl)amide (47 mL of a 1M solution in THF) was added dropwise. An exotherm was observed and the solution became brown in colour. Stirring was continued at −50° C. for 1 h, then allowed to warm to room temperature and stirred overnight. The reaction mixture was slowly poured into water (700 mL). The aqueous phase was extracted with ethyl acetate. The combined extracts were washed with brine, dried over sodium sulphate and concentrated under reduced pressure to give the crude product. Purification by flash chromatography (SiO₂, ethyl acetate/cyclohexane gradient) furnished tert-butyl (1R, 5S)-3-cyano-3-(5-fluoro-3-pyridyl)-8-azabicyclo [3.2.1] oct-6-ene-8-carboxylate as a white solid.

LC MS (method I): 330 [M+H]⁺. 1H NMR (400 MHz, CDCl₃, TMS) δ/ppm: 1.46-1.50 (m, 2H), 1.52 (s, 9H), 2.11-2.30 (m, 3H), 2.34-2.49 (m, 1H), 4.67-4.86 (m, 2H), 6.36-6.48 (m, 2H), 7.50-7.57 (m, 1H), 8.43-8.48 (m, 1H) 8.53-8.58 (m, 1H).

### Step 2: Preparation of (1R,5S)-3-(5-fluoro-3-pyridyl)-8-azoniabicyclo[3.2.1]oct-6-ene-3-carbonitrile TFA salt

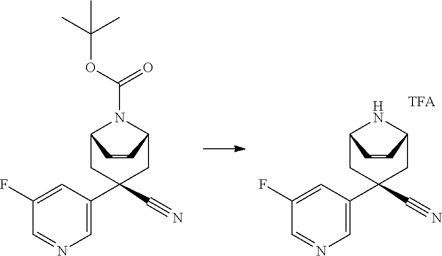 [SMILES: CC(C)(C)OC(=O)N1[C@@H]2C=C[C@H]1C[C@](C#N)(c1cncc(F)c1)C2 ; N#C[C@@]1(c2cncc(F)c2)C[C@H]2C=C[C@@H](C1)N2]

2,2,2-Trifluoroacetic acid (23 mL, 300 mmol) was slowly added to a solution of tert-butyl (1R,5S)-3-cyano-3-(5-fluoro-3-pyridyl)-8-azabicyclo[3.2.1]oct-6-ene-8-carboxylate (9.8 g, 30 mmol) in dichloromethane (100 mL) at 20° C. After completion of the addition, stirring was continued overnight. The reaction mixture was evaporated under reduced pressure to give brown oil which was triturated with diethyl ether (200 mL) to furnish (1R,5S)-3-(5-fluoropyridin-1-ium-3-yl)-8-azoniabicyclo[3.2.1]oct-6-ene-3-carbonitrile as 2,2,2-trifluoroacetate salt as a light brown solid.

¹H NMR (400 MHz, DMSO-d₆, TMS) δ/ppm: 2.41-2.48 (m, 2H), 2.56-2.65 (m, 2H), 4.62-4.69 (m, 2H), 6.46-6.52 (m, 2H), 7.99-8.06 (m, 1H), 8.64-8.67 (m, 1H), 8.70-8.74 (m, 1H).

### Step 3: Preparation of (1R,5S)-8-(2,2-difluoroethyl)-3-(5-fluoro-3-pyridyl)-8-azabicyclo[3.2.1]oct-6-ene-3-carbonitrile

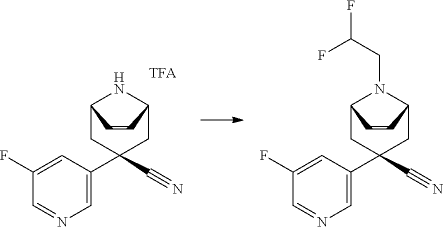 [SMILES: N#C[C@@]1(c2cncc(F)c2)C[C@H]2C=C[C@@H](C1)N2 ; N#C[C@@]1(c2cncc(F)c2)C[C@H]2C=C[C@@H](C1)N2CC(F)F]

To a stirred solution of 3-(5-fluoro-3-pyridyl)-8-azabicyclo[3.2.1]oct-6-ene-3-carbonitrile (2,2,2-trifluoroacetate salt) (0.900 g, 1.97 mmol) in dimethylformamide (20 mL), diisopropylethyl amine (0.866 mL, 4.92 mmol) was added dropwise under nitrogen and stirred for 15 min. 2,2-Difluoroethyl trifluoromethanesulfonate (0.632 g, 2.95 mmol) was then added dropwise and stirred at room temperature overnight. The reaction mixture was quenched with water (50 mL) and extracted with ethyl acetate (3×30 mL). The combined extracts were washed with brine, dried over anhydrous sodium sulfate and concentrated under reduced pressure to give the crude product. Purification by flash chromatography (SiO₂, ethyl acetate/cyclohexane gradient) furnished (1R,5S)-8-(2,2-difluoroethyl)-3-(5-fluoro-3-pyridyl)-8-azabicyclo[3.2.1]oct-6-ene-3-carbonitrile as an off white solid (m.p. 109-111° C.).

LC MS (method I): 294 [M+H]⁺.

¹H NMR (400 MHz, CDCl₃, TMS) δ/ppm: 2.23-2.47 (m, 4H), 2.67-2.88, (m, 2H), 3.85 (br s, 2H), 5.67-6.11 (m, 1H), 6.34 (s, 2H) 7.52-7.67 (m, 1H), 8.45 (d, J=2.5 Hz, 1H), 8.66 (br s, 1H).

### Step 4: Preparation (1R,5S)-8-(2,2-difluoroethyl)-3-(5-fluoro-3-pyridyl)-8-azabicyclo[3.2.1]oct-6-ene-3-carbothioamide (Compound E.071)

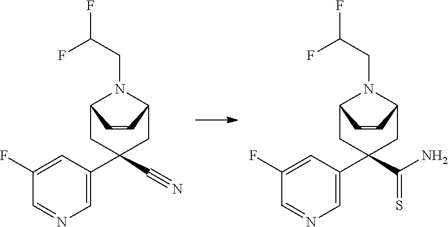 [SMILES: N#C[C@@]1(c2cncc(F)c2)C[C@H]2C=C[C@@H](C1)N2CC(F)F ; NC(=S)[C@@]1(c2cncc(F)c2)C[C@H]2C=C[C@@H](C1)N2CC(F)F]

(1R,5S)-8-(2,2-Difluoroethyl)-3-(5-fluoro-3-pyridyl)-8-azabicyclo[3.2.1]oct-6-ene-3-carbonitrile (200 mg, 0.68 mmol) was dissolved in a mixture of 1,4-dioxane (2.5 mL) and water (0.5 mL). O,O′-diethyldithiophosphate (0.38 mL, 2.05 mmol) was added dropwise to the reaction mixture at room temperature and then refluxed at 80° C. for 20 h. The reaction mixture was cooled to room temperature and poured into saturated sodium carbonate (20 mL). It was stirred for 30 min and organic layer was separated. The aqueous layer was extracted with ethyl acetate (3×20 mL). The combined extracts were washed with brine, dried over anhydrous sodium sulfate concentrated under reduced pressure to give the crude product. Purification by flash chromatography (SiO₂, dichloromethane/methanol gradient) furnished (1R,5S)-8-(2,2-difluoroethyl)-3-(5-fluoro-3-pyridyl)-8-azabicyclo[3.2.1]oct-6-ene-3-carbothioamide (m.p. 244-246° C.).

LC MS (method I): 328 [M+H]⁺.

¹H NMR (400 MHz, DMSO-d₆, TMS) δ/ppm: 2.02-2.09 (m, 2H), 2.59 (td, J=15.8, 4.4 Hz, 2H), 3.32 (s, 2H), 3.54 (d, J=13.8 Hz, 2H), 3.63 (br s, 2H), 5.84-6.17 (m, 3H), 7.51-7.66 (m, 1H), 8.36-8.50 (m, 2H), 8.99 (s, 1H), 9.49 (s, 1H).

### Example P21

### Preparation of (1 S,5R)-8-(2,2-difluoroethyl)-3-(5-iodo-3-pyridyl)-8-azabicyclo[3.2.1]octane-3-carbothioamide (Compound E.072)

### Step 1: Preparation of tert-butyl (1 S,5R)-3-cyano-3-(5-iodo-3-pyridyl)-8-azabicyclo[3.2.1]octane-8-carboxylate

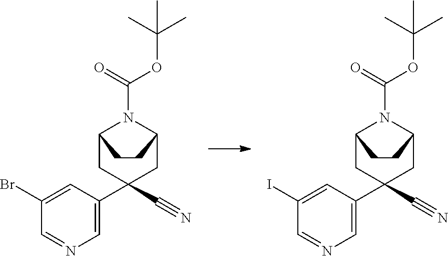 [SMILES: CC(C)(C)OC(=O)N1[C@@H]2CC[C@H]1C[C@](C#N)(c1cncc(Br)c1)C2 ; CC(C)(C)OC(=O)N1[C@@H]2CC[C@H]1C[C@](C#N)(c1cncc(I)c1)C2]

Butyl lithium (13 mL of a 1M solution in THF) was added dropwise to a stirred solution of tert-butyl (1S,5R)-3-cyano-3-(5-iodo-3-pyridyl)-8-azabicyclo[3.2.1]octane-8-carboxylate (7.0 g, 17.8 mmol) in tetrahydrofuran (50 mL) at −60° C. over 15 min under nitrogen atmosphere. The reaction mixture turned immediately yellow. Stirring was continued at −60° C. for 30 min, and then molecular iodine (6.7 g, 26.8 mmol) dissolved in tetrahydrofuran was added. The reaction mixture is then allowed to warm to room temperature during 4 h. The reaction mixture was then poured into cold ammonium chloride solution and extracted with ethyl acetate (3×). The combined extracts were washed with brine, dried (Na₂SO₄) and evaporated under reduced pressure to give the crude product as a solid.

LC MS (method I): 440 [M+H]⁺.

¹H NMR (400 MHz, CDCl₃, TMS) δ/ppm: 1.24-1.29 (m, 2H), 1.50 (d, J=2.0 Hz, 3H), 1.51 (s, 10H), 2.14-2.31 (m, 8H), 2.35-2.44 (m, 5H), 3.14-3.26 (m, 1H), 4.28-4.55 (m, 3H), 8.08 (t, J=2.1 Hz, 1H), 8.68 (d, J=2.2 Hz, 1H), 8.79 (d, J=1.8 Hz, 1H).

### Step 2: Preparation of Preparation of (1 S,5R)-3-(5-iodo-3-pyridyl)-8-azoniabicyclo[3.2.1]octane-3-carbonitrile (TFA salt)

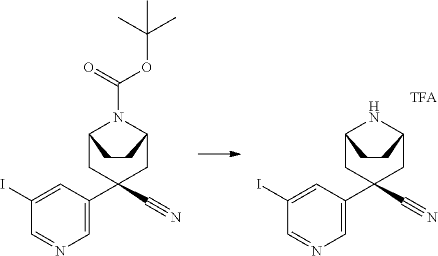 [SMILES: CC(C)(C)OC(=O)N1[C@@H]2CC[C@H]1C[C@](C#N)(c1cncc(I)c1)C2 ; N#C[C@@]1(c2cncc(I)c2)C[C@H]2CC[C@@H](C1)N2]

2,2,2-Trifluoroacetic acid (12.2 mL, 159 mmol) was slowly added to a solution (1R,5S)-3-cyano-3-(5-iodo-3-pyridyl)-8-azabicyclo[3.2.1]octane-8-carboxylate (7.00 g, 15.9 mmol) in dichloromethane (55 mL) at 20° C. After completion of the addition, stirring was continued overnight. The reaction mixture was concentrated and solid obtained was then triturated in ether, concentrated under vacuum to furnish (1S,5R)-3-(5-iodo-3-pyridyl)-8-azoniabicyclo[3.2.1]octane-3-carbonitrile as a 2,2,2-trifluoroacetate salt.

LC MS (method I): 340 [M+H]⁺.

### Step 3: Preparation of (1 S,5R)-8-(2,2-difluoroethyl)-3-(5-iodo-3-pyridyl)-8-azabicyclo[3.2.1]octane-3-carbonitrile

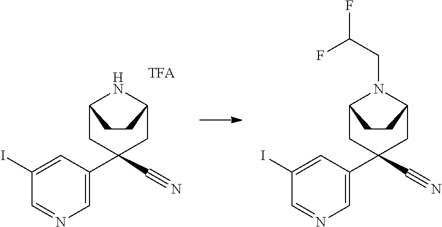 [SMILES: N#C[C@@]1(c2cncc(I)c2)C[C@H]2CC[C@@H](C1)N2 ; N#C[C@@]1(c2cncc(I)c2)C[C@H]2CC[C@@H](C1)N2CC(F)F]

A round bottom flask was charged with 3-(5-iodo-3-pyridyl)-8-azoniabicyclo[3.2.1]octane-3-carbonitrile 2,2,2-trifluoroacetate salt (4.00 g, 7.05 mmol) in dichloromethane (40 mL) followed by dropwise addition of N,N-ethyldiisopropylamine (1.80 g, 14.1 mmol) and stirred for 10 min. To this suspension, 2,2-difluoroethylmethanesulfonate (3.00 g, 14.1 mmol) was added dropwise at room temperature under nitrogen atmosphere. The reaction mixture was stirred at room temperature for 24 h. The reaction mixture was poured into saturated bicarbonate solution and the resulting mixture was extracted with dichloromethane (3×). The organic layer was separated and washed with water and brine. After drying with sodium sulfate, the organic layer was filtered and concentrated in vacuum. Purification by flash chromatography (SiO₂, ethyl acetate/cyclohexane gradient) furnished (1S,5R)-8-(2,2-difluoroethyl)-3-(5-iodo-3-pyridyl)-8-azabicyclo[3.2.1]octane-3-carbonitrile as a pale yellow solid (m.p. 117-119° C.).

LC MS (method I): 404 [M+H]⁺.

¹H NMR (400 MHz, CDCl₃, TMS) δ/ppm: 0.90-1.01 (m, 1H), 1.34-1.44 (m, 1H), 1.58 (br s, 1H), 2.02-2.20 (m, 2H), 2.30 (br s, 4H), 2.39 (br s, 2H), 2.63-2.91 (m, 2H), 3.46 (br s, 2H), 5.61-6.16 (m, 1H), 8.14 (br s, 1H), 8.62-8.90 (m, 2H).

### Step 4: Preparation of (1 S,5R)-8-(2,2-difluoroethyl)-3-(5-iodo-3-pyridyl)-8-azabicyclo[3.2.1]octane-3-carbothioamide (Compound E.072)

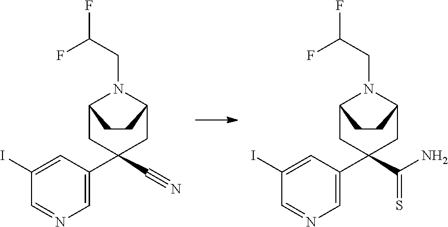 [SMILES: N#C[C@@]1(c2cncc(I)c2)C[C@H]2CC[C@@H](C1)N2CC(F)F ; NC(=S)[C@@]1(c2cncc(I)c2)C[C@H]2CC[C@@H](C1)N2CC(F)F]

(1S,5R)-8-(2,2-difluoroethyl)-3-(5-iodo-3-pyridyl)-8-azabicyclo[3.2.1]octane-3-carbonitrile (1.00 g, 1.24 mmol) was dissolved in pyridine (20 mL) and treated with 45% (by weight) solution of ammonium sulfide in water (6 equiv., 7.44 mmol) and stirred at room temperature for 40 h. The reaction mixture was added slowly into ice water and stirred for 15 min. The aqueous layer was extracted with dichloromethane (3×50 ml). The combined organic layer was washed with brine, dried over anhydrous sodium sulfate, filtered and concentrated under reduced pressure. The solid obtained was then triturated in ether to obtain the title compound as a solid (m.p. 189-191° C.).

¹H NMR (400 MHz, DMSO-d6) δ/ppm: 1.65-1.75 (m, 2H), 1.93 (d, J=7.5 Hz, 2H), 2.04 (br s, 2H), 2.56-2.75 (m, 2H), 3.24 (br s, 2H), 3.41-3.57 (m, 2H), 5.75-6.20 (m, 1H), 8.08 (t, J=2.0 Hz, 1H), 8.50-8.71 (m, 2H), 9.26 (br s, 1H), 9.86 (s, 1H).

### Example P22

### Preparation of tert-butyl (1S,5R)-3-(5-cyclopropyl-3-pyridyl)-8-(2,2,2-trifluoroethyl)-8-azabicyclo[3.2.1]octane-3-carbothioamide (Compound E.023)

### Step 1: Preparation of tert-butyl (1 S,5R)-3-cyano-3-(5-cyclopropyl-3-pyridyl)-8-azabicyclo[3.2.1]octane-8-carboxylate

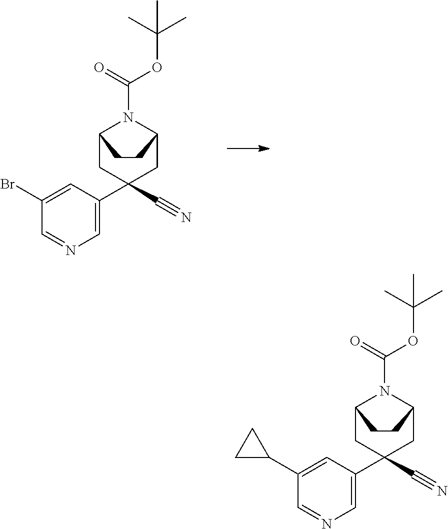 [SMILES: CC(C)(C)OC(=O)N1[C@@H]2CC[C@H]1C[C@](C#N)(c1cncc(Br)c1)C2 ; CC(C)(C)OC(=O)N1[C@@H]2CC[C@H]1C[C@](C#N)(c1cncc(C3CC3)c1)C2]

tert-Butyl (1S,5R)-3-(5-bromo-3-pyridyl)-3-cyano-8-azabicyclo[3.2.1]octane-8-carboxylate (4.00 g, 10.2 mmol) is dissolved in toluene (30 mL) and treated with tricyclohexylphosphine (0.29 g, 1.02 mmol), potassium phosphate (7.80 g, 35.7 mmol), cyclopropyl boronic acid (1.3 g, 15.3 mmol) and water (1.6 mL). Then palladium acetate (0.114 g, 0.509 mmol) was added and the reaction mixture was heated to 100° C. for 20 h. The reaction mixture was then poured into cold ammonium chloride solution and extracted with ethyl acetate (3×). The combined extracts were washed with brine, dried over sodium sulfate and evaporated under reduced pressure to give the crude product. Purification by flash chromatography (SiO₂, ethyl acetate/cyclohexane gradient) furnished the titel compound as a solid.

LC MS (method I): 354 [M+H]⁺.

### Step 2: Preparation of (1S,5R)-3-(5-cyclopropyl-3-pyridyl)-8-azabicyclo[3.2.1]octane-3-carbonitrile (TFA salt)

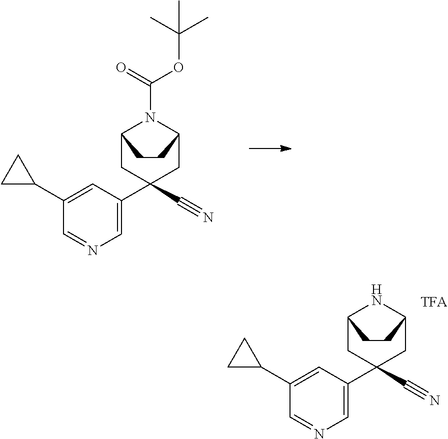 [SMILES: CC(C)(C)OC(=O)N1[C@@H]2CC[C@H]1C[C@](C#N)(c1cncc(C3CC3)c1)C2 ; N#C[C@@]1(c2cncc(C3CC3)c2)C[C@H]2CC[C@@H](C1)N2]

2,2,2-Trifluoroacetic acid (4.20 mL, 54.0 mmol) was slowly added to a solution of tert-butyl (1S,5R)-3-cyano-3-(5-cyclopropyl-3-pyridyl)-8-azabicyclo[3.2.1]octane-8-carboxylate (1.90 g, 5.40 mmol) in dichloromethane (20 mL) at 20° C. After completion of the addition, stirring was continued overnight. The reaction mixture was concentrated and the solid obtained was then triturated in ether, concentrated under vacuum to furnish the title compound as 2,2,2-trifluoroacetate salt).

LC MS (method I): 254 [M+H]+.

### Step 3: Preparation of (1 S,5R)-3-(5-cyclopropyl-3-pyridyl)-8-(2, 2, 2-trifluoroethyl)-8-azabicyclo[3.2.1]octane-3-carbonitrile

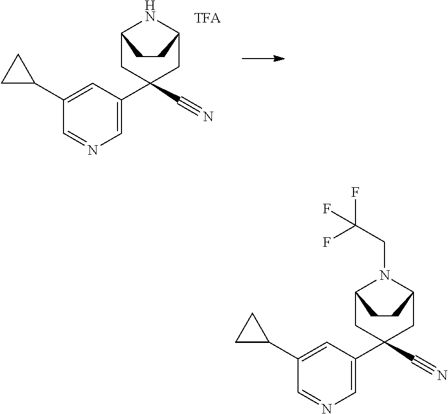 [SMILES: N#C[C@@]1(c2cncc(C3CC3)c2)C[C@H]2CC[C@@H](C1)N2 ; N#C[C@@]1(c2cncc(C3CC3)c2)C[C@H]2CC[C@@H](C1)N2CC(F)(F)F]

A round bottom flask was charged with (1S,5R)-3-(5-cyclopropyl-3-pyridyl)-8-azabicyclo[3.2.1]octane-3-carbonitrile (TFA salt) (500 mg, 1.04 mmol) in N,N-dimethylformamide (2.0 mL) followed by catalytic sodium iodide and potassium carbonate (0.581 g, 4.163 mmol) and stirred for 10 min. To this suspension was added dropwise 2,2,2-trifluoroethyl trifluoromethanesulfonate (2 equiv) at room temperature under nitrogen atmosphere. The reaction mixture was then stirred at room temperature for 20 h and filtered through Celite and diluted in water. The resulting mixture was extracted with ethyl acetate (3×), the organic layer was separated and washed with water and brine. After drying over sodium sulfate, the organic layer was filtered and concentrated in vacuum. Purification by flash chromatography (SiO₂, ethyl acetate/cyclohexane gradient) furnished the title compound as a solid.

LC MS (method I): 336 [M+H]⁺.

¹H NMR (400 MHz, CDCl₃, TMS) δ/ppm 0.78-0.82 (m, 2H), 1.09-1.18 (m, 2H), 1.96 (m, 1H), 2.07-2.11 (m, 2H), 2.30-2.47 (m, 4H), 2.88-2.95 (q, 2H), 3.49-3.50 (m, 2H), 7.47-7.48 (m, 1H), 8.35-8.36 (d, 1H), 8.57-8.58 (d, 1H).

### Step 4: Preparation of tert-butyl (1 S,5R)-3-(5-cyclopropyl-3-pyridyl)-8-(2,2,2-trifluoroethyl)-8-azabicyclo[3.2.1]octane-3-carbothioamide (Compound E.023)

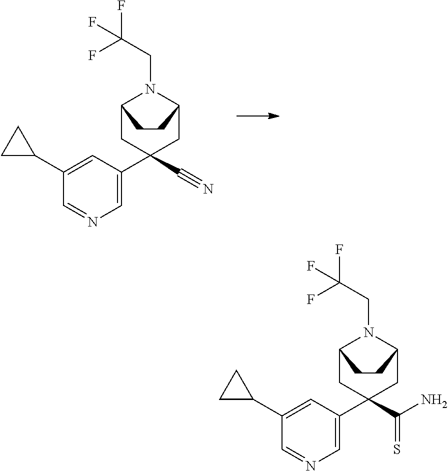 [SMILES: N#C[C@@]1(c2cncc(C3CC3)c2)C[C@H]2CC[C@@H](C1)N2CC(F)(F)F ; NC(=S)[C@@]1(c2cncc(C3CC3)c2)C[C@H]2CC[C@@H](C1)N2CC(F)(F)F]

(1 S,5R)-3-(5-cyclopropyl-3-pyridyl)-8-(2, 2, 2-trifluoroethyl)-8-azabicyclo[3.2.1]octane-3-carbonitrile (0.150 g, 0.445 mmol) was dissolved in ethanol (10 mL) and treated with phosphorouspentasulfide (1.78 mmol) and stirred at room temperature overnight. Reaction mixture was added slowly into water. The aqueous layer was extracted with ethyl acetate (3×50 mL). The combined organic layer was washed with brine, dried over anhydrous sodium sulfate, filtered and concentrated under reduced pressure. Purification by flash chromatography (SiO₂, ethyl acetate/cyclohexane gradient) furnished the title compound as a solid.

LC MS (method I): 370 [M+H]+.

The compounds in the following tables can be prepared analogously. The examples which follow are intended to illustrate the invention and show preferred compounds of formula (I).

TABLE E1
Physical data of compounds of formula (I)

| | | | | | | | (I) |
|---|---|---|---|---|---|---|---|
| 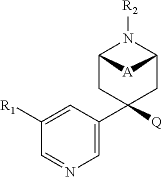 | | | | | | | |
| | | | | | | LCMS | LCMS |
| | | | | | m.p. | ret. time | m/z |
| No. | Q | A | R¹ | R² | (° C.) | (min/method) | [M + H]⁺ |
| E.001 | —C(S)NH₂ | —CH═CH— | Cl | 2,2-difluoroethyl | 241-242 | | |
| E.002 | —C(S)NH₂ | —CH₂—CH₂— | Cl | 2,2,2-trifluoroethyl | 223-224 | | |
| E.003 | —C(S)NH₂ | —CH₂—CH₂— | Cl | H | 219-221 | | |
| E.004 | —C(S)NH₂ | —CH₂—CH₂— | Cl | Me | 210-212 | | |
| E.005 | —C(S)NH₂ | —CH₂—CH₂— | Br | CH(Me)C(O)OMe | 141-145 | | |
| E.006 | —C(S)NH₂ | —CH₂—CH₂— | Br | 2-chloroallyl | 146-153 | | |
| E.007 | —C(S)NH₂ | —CH₂—CH₂— | Br | 2,2,2-trifluoroethyl | 234-236 | | |
| E.008 | —C(S)NH₂ | —CH═CH— | Br | 4,4,4-trifluorobutyl | 200-202 | | |
| E.009 | —C(S)NH₂ | —CH₂—CH₂— | CN | 2,2,2-trifluoroethyl | 180-186 | | |
| E.010 | —C(S)NH₂ | —CH₂—CH₂— | Br | H | 164-168 | | |
| E.011 | —C(S)NH₂ | —CH═CH— | Br | propargyl | 233-235 | | |
| E.012 | —C(S)NH₂ | —CH═CH— | CN | 2-chloroallyl | | 0.35/G | 345 |
| E.013 | —C(S)NH₂ | —CH═CH— | Br | 2-chloroallyl | | 0.59/G | 398 |
| E.014 | —C(NH)SMe | —CH₂—CH₂— | Cl | 2,2,2-trifluoroethyl | | 1.34/G | 378 |
| | | | | | | | [M − H]⁻ |
| E.015 | —C(S)NH₂ | —CH═CH— | Br | H | 225-227 | | |
| E.016 | —C(S)NH₂ | —CH═CH— | Br | 1-methylprop-2-ynyl | 208-210 | | |
| E.017 | —C(S)NH₂ | —CH₂—CH₂— | CN | H | 135-136 | | |
| E.018 | —C(S)NH₂ | —CH═CH— | Cl | H | 175-177 | | |
| E.019 | —C(S)NH₂ | —CH₂—CH₂— | Cl | 1-methylprop-2-ynyl | 215-217 | | |
| E.020 | —C(S)NH₂ | —CH═CH— | Br | 2,2,2-trifluoroethyl | 251-253 | | |
| E.021 | —C(S)NH₂ | —CH₂—CH₂— | Br | tert-butoxycarbonyl | 228-230 | | |
| E.022 | —C(S)NH₂ | —CH₂—CH₂— | Cl | tert-butoxycarbonyl | 199-200 | | |
| E.023 | —C(S)NH₂ | —CH₂—CH₂— | c-Pr | 2,2,2-trifluoroethyl | 215-217 | | |
| E.024 | —C(S)NH₂ | —CH₂—CH₂— | MeO | 2,2,2-trifluoroethyl | 178-180 | | |
| E.025 | —C(S)NH₂ | —CH₂—CH₂— | CN | 4,4,4-trifluorobutyl | 167-168 | | |
| E.026 | —C(S)NH₂ | —CH₂—CH₂— | CN | 2-methoxyethyl | 174-177 | | |
| E.027 | —C(S)NH₂ | —CH₂—CH₂— | Cl | 2-methoxyethyl | 195-199 | | |
| E.028 | —C(S)NH₂ | —CH₂—CH₂— | Cl | cyclopropyl | 228-231 | | |
| E.029 | —C(S)NH₂ | —CH₂—CH₂— | Cl | 4,4,4-trifluorobutyl | 189-191 | | |
| E.030 | —C(S)NH₂ | —CH₂—CH₂— | Cl | CH(Me)C(O)OMe | 178-180 | | |
| E.031 | —C(S)NH₂ | —CH₂—CH₂— | Br | 4,4,4-trifluorobutyl | 185-187 | | |
| E.032 | —C(S)NH₂ | —CH₂—CH₂— | Br | 2-methoxyethyl | 183-184 | | |
| E.033 | —C(S)NH₂ | —CH₂—CH₂— | Cl | 2-methylallyl | 199-201 | | |
| E.034 | —C(S)NH₂ | —CH═CH— | I | H | 163-164 | | |
| E.035 | —C(S)NH₂ | —CH═CH— | Br | 2-methyl- | 240-241 | | |
| | | | | sulfanylethyl | | | |
| E.036 | —C(S)NH₂ | —CH₂—CH₂— | Br | propargyl | 205-206 | | |
| E.037 | —C(S)NH₂ | —CH₂—CH₂— | Br | cyclopropyl | 213-214 | | |
| E.038 | —C(S)NH₂ | —CH₂—CH₂— | Br | 2-methylallyl | 174-175 | | |
| E.039 | —C(S)NH₂ | —CH═CH— | I | 2,2-difluoroethyl | 224-225 | | |
| E.040 | —C(S)NH₂ | —CH═CH— | Br | thietan-3-yl | 224-225 | | |
| E.041 | —C(S)NH₂ | —CH═CH— | CN | 2,2-difluoroethyl | 188-189 | | |
| E.042 | —C(S)NH₂ | —CH═CH— | CN | 2-methyl- | 194-195 | | |
| | | | | sulfanylethyl | | | |
| E.043 | —C(S)NH₂ | —CH₂—CH₂— | Br | 2,2-difluoroethyl | 227-228 | | |
| E.044 | —C(S)NH₂ | —CH═CH— | Br | 2-methylallyl | 210-211 | | |
| E.045 | —C(S)NH₂ | —CH═CH— | Br | cyclopropyl | 274-275 | | |
| E.046 | —C(S)NH₂ | —CH₂—CH₂— | Cl | 2,2-difluoroethyl | 202-203 | | |
| E.047 | —C(S)NH₂ | —CH═CH— | CN | cyclopropyl | 245-246 | | |
| E.048 | —C(S)NH₂ | —CH₂—CH₂— | Cl | 2,2-difluoropropyl | 200-201 | | |
| E.049 | —C(S)NH₂ | —CH₂—CH₂— | Cl | 2,2-difluorobutyl | 202-203 | | |
| E.050 | —C(S)NH₂ | —CH═CH— | Br | 2-methoxyethyl | 171-173 | | |
| E.051 | —C(S)NH₂ | —CH═CH— | Br | CH(Me)C(O)OMe | 165-166 | | |
| E.052 | —C(S)NH₂ | —CH₂—CH₂— | Br | CH(Me)C(O)OMe | 176-177 | | |
| E.053 | —C(S)NH₂ | —CH₂—CH₂— | Br | 2,2-difluoropropyl | 210-211 | | |
| E.054 | —C(S)NH₂ | —CH₂—CH₂— | CN | cyclopropyl | 209-211 | | |
| E.055 | —C(S)NH₂ | —CH₂—CH₂— | Cl | 2-methyl- | 173-175 | | |
| | | | | sulfanylethyl | | | |
| E.056 | —C(S)NH₂ | —CH₂—CH₂— | Cl | propargyl | 206-208 | | |
| E.057 | —C(S)NH₂ | —CH═CH— | Br | allyl | 183-185 | | |
| E.058 | —C(S)NH₂ | —CH₂—CH₂— | Br | 2-methyl- | 178-180 | | |
| | | | | sulfanylethyl | | | |
| E.059 | —C(S)NH₂ | —CH═CH— | Br | 2,2-difluoropropyl | 217-219 | | |
| E.060 | —C(S)NH₂ | —CH═CH— | I | 2,2,2-trifluoroethyl | 236-238 | | |
| E.061 | —C(S)NH₂ | —CH═CH— | CN | allyl | 174-175 | | |
| E.062 | —C(S)NH₂ | —CH═CH— | Br | 2,2-difluoroethyl | 248-249 | | |
| E.063 | —C(S)NH₂ | —CH═CH— | I | allyl | 186-187 | | |
| E.064 | —C(S)NH₂ | —CH═CH— | CN | 4,4,4-trifluorobutyl | 197-198 | | |
| E.065 | —C(S)NH₂ | —CH₂—CH₂— | CN | CH(Me)C(O)OMe | 78-79 | | |
| E.066 | —C(S)NH₂ | —CH═CH— | CN | 2,2,2-trifluoroethyl | 185-187 | | |
| E.067 | —C(S)NH₂ | —CH═CH— | Cl | 4,4,4-trifluorobutyl | 199-201 | | |
| E.068 | —C(S)NH₂ | —CH═CH— | ¹⁾ | 2,2,2-trifluoroethyl | 179-180 | | |
| E.069 | —C(S)NH₂ | —CH═CH— | CN | 2-methoxyethyl | 197-198 | | |
| E.070 | —C(S)NH₂ | —CH═CH— | ²⁾ | 2,2,2-trifluoroethyl | 210-213 | | |
| E.071 | —C(S)NH₂ | —CH═CH— | F | 2,2-difluoroethyl | 244-246 | | |
| E.072 | —C(S)NH₂ | —CH₂—CH₂— | I | 2,2-difluoroethyl | 190-191 | | |
| E.073 | —C(S)NH₂ | —CH₂—CH₂— | CN | 2,2-difluoroethyl | 191-193 | | |

¹⁾ R¹ = —CEC—SiMe₃;
²⁾ R¹ = —CEC—H

### Biological Examples

These examples illustrate the pesticidal/insecticidal properties of compounds of formula I.

Tests were performed as follows:

### Example B1

### Activity Against Myzus persicae (Green Peach Aphid)

Sunflower leaf discs were placed on agar in a 24-well microtiter plate and sprayed with the test solutions at an application rate of 200 ppm. After drying, the leaf discs were infested with an aphid population of mixed ages. After an incubation period of 6 days, samples were checked for mortality.

The following compounds gave at least 80% control of Myzus persicae:

E.001, E.002, E.003, E.004, E.005, E.006, E.007, E.008, E.009, E.010, E.011, E.013, E.014, E.015, E.016, E.017, E.018, E.019, E.020, E.023, E.024, E.025, E.026, E.027, E.028, E.029, E.030, E.031, E.032, E.033, E.034, E.035, E.036, E.037, E.038, E.039, E.040, E.041, E.042, E.043, E.044, E.045, E.046, E.047, E.048, E.049, E.050, E.051, E.052, E.053, E.054, E.055, E.056, E.057, E.058, E.059, E.060, E.061, E.062, E.063, E.064, E.065, E.066, E.067, E.068, E.069, E.070, E.071, E.072, E.073.

### Example B2

### Activity Against Myzus persicae (Green Peach Aphid)

Pea seedlings, infested with an aphid population of mixed ages, were placed with the roots directly in the test solutions at an application rate of 24 ppm. 6 days after introduction, samples were checked for mortality.

The following compounds gave at least 80% control of Myzus persicae:

E.001, E.003, E.004, E.005, E.006, E.008, E.010, E.011, E.012, E.018, E.019, E.025, E.026, E.027, E.028, E.029, E.030, E.031, E.032, E.033, E.035, E.036, E.037, E.038, E.039, E.040, E.041, E.042, E.043, E.044, E.045, E.046, E.047, E.049, E.050, E.051, E.052, E.053, E.054, E.055, E.056, E.057, E.058, E.059, E.061, E.063, E.064, E.065, E.066, E.067, E.069, E.071, E.072, E.073.

### Example B3

### Activity Against Bemisia tabaci (Cotton White Fly)

Cotton leaf discs were placed on agar in a 24-well microtiter plate and sprayed with test solutions at an application rate of 200 ppm. After drying, the leaf discs were infested with 12 to 18 adults. After an incubation period of 6 days after infestation, samples were checked for mortality.

The following compounds gave at least 80% control of Bemisia tabaci:

E.001, E.002, E.003, E.004, E.005, E.006, E.007, E.008, E.009, E.010, E.011, E.013, E.014, E.015, E.016, E.017, E.018, E.020, E.023, E.024, E.025, E.026, E.027, E.028, E.029, E.030, E.031, E.032, E.033, E.034, E.035, E.036, E.037, E.038, E.039, E.040, E.041, E.042, E.043, E.044, E.045, E.046, E.047, E.048, E.049, E.050, E.051, E.052, E.053, E.054, E.055, E.056, E.057, E.058, E.059, E.060, E.061, E.062, E.063, E.064, E.065, E.066, E.071, E.072, E.073.

### Example B4

### Control of Insects Resistant to Neonicotinoids

The level of resistance and therefore the impact on the performance of the insecticide can be measured by the use of a ‘Resistance Factor’. The resistance factor can be calculated by dividing the concentration of an insecticide that provides a set level of mortality (i.e. 80%) for the ‘resistant’ strain with the concentration of the same insecticide that provides the same level of mortality for the ‘susceptible’ insect of the same species and life-stage. Although there are no set rules, a low value (less than or equal to 20) indicates no cross-resistance and only natural levels of variation and a high value (greater than or equal to 64) provides strong evidence of cross-resistance.

In order to obtain neonicotinoid resistant insects, a researcher is to locate a host crop and geographical region where the relevant resistance had been reported in literature (e.g. Myzus persicae—peach orchards of France. Bemisia tabaci—protected vegetables in Spain). Live samples of the insect are then collected from the locations/host crops and transported back to a laboratory, where breeding colonies would be established. Non-resistant individuals with the colonies are eliminated to provide a homologous-resistant population. This is achieved by either establishing a clonal population of insects from a single resistant individual (e.g. Myzus persicae) or by repeatedly exposing the colony to a dose of insecticide which kills susceptible insects, whilst leaving resistant insects unaffected. The resistant phenotype of the insect colony is determined either by conducting a full dose response bioassay (examples of which can be found on the IRAC web-site and below) with a neonicotinoid insecticide and comparing the bioassay results to similar bioassay results for a known susceptible colony of the same species. Alternatively the resistance genotype of the individual insects can be determined by molecular techniques (e.g. PCR) if the resistance mechanism for the relevant species is known.

a) Neonicotinoid Resistant Strain of the Green Peach Aphid (Myzus persicae)
a.1) Myzus persicae Strains Utilised:
Standard screening strain of Myzus persicae (Neonicotinoid susceptible)
FRC-P strain of Myzus persicae (Neonicotinoid resistant) obtained from peach orchards in Southern France

### a2) Bioassay Methods Utilised

### a.2.1) Bioassay, Method A:

Myzus persicae: Mixed Population, Contact Activity, Curative on Pea Seedlings

Pea seedlings were infested with an aphid population of mixed ages and treated with the test solutions in a spray chamber. 6 days after treatment samples were checked for mortality.

Application rates: 200 ppm, 50 ppm, 12.5 ppm, 3 ppm and 0.8 ppm.

### a.2.2) Dose-Response Bioassay, Method B:

Test pots (45 mm diameter) were prepared with discs of Chinese cabbage on tap water agar adapted from Herron et al (Aust J Entomol 37:70-73 (1998)). Mixed age aphids (numbering 20-30) were transferred to the dishes and allowed to settle for 24 h at 21 degrees C. with a 16:8 h light/dark regime. Dead individuals were removed prior to application. Serial dilutions of insecticide were applied using a Potter precision laboratory spray tower (Burkard Scientific, Uxbridge, UK), before sealing each pot with a lid. Each treatment replicate was sprayed with 3 mL solution at 0.6 bar with a 3 s settling time (equivalent to approximately 400 L ha-1). A minimum of five insecticide concentrations and three replicates per treatment were utilised in each test. Aphid mortality is assessed at 72 hours after treatment (depending on insecticide mode of action). LC50 values were calculated by LOGIT analysis (using ACSAPwin program).

### a.3) Results

The following compounds, according to the present invention, gave at least 80% control of the FRC-P (Neonicotinoid resistant) strain of Myzus persicae at 200 ppm and exhibited a resistance factor of ≦20: E.011, E.012.

Thiacloprid and Imidacloprid failed to give 80% control of the FRC-P (Neonicotinoid resistant) strain of Myzus persicae at 200 ppm and both exhibited a Resistance Factor (RF₈₀) of >64.

b) Neonicotinoid and Pyrethroid Resistant Strain of the Tobacco Whitefly (Bemisia tabaci)
b.1) Bemisia tabaci Strains Utilised:
Standard screening strain of Bemisia tabaci (Neonicotinoid susceptible)
Q-biotype strain of Bemisia tabaci (Neonicotinoid resistant) originally provided by Rothamsted Research, UK.

### b.2) Bioassay Methods Utilised:

### b.2.1) Bioassay, Method A:

Bemisia tabaci: Residual Activity, Preventive Egg Lay

Cotton seedlings, with all but a single leaf removed are treated with the diluted test solutions in a turn table spray chamber. 24 hours after drying, they are infested with 20 adult whitefly. 3 days after exposure, the total number of adult whitefly and the total number of whitefly eggs laid on the leaf are counted. Percentage control of egg lay is calculated and corrected for control mortality.

Application rates: 200 ppm, 50 ppm, 12.5 ppm, 3 ppm and 0.8 ppm.

### b.2.2) Dose-Response Bioassay, Method B:

Test pots (45 mm diameter) were prepared with discs of cotton leaf on tap water agar adapted from Herron et al (Aust J Entomol 37:70-73 (1998)). Serial dilutions of insecticide were applied using a Potter precision laboratory spray tower (Burkard Scientific, Uxbridge, UK). Each treatment replicate was sprayed with 3 mL solution at 0.6 bar with a 3 s settling time (equivalent to approximately 400 L ha-1). A minimum of five insecticide concentrations and three replicates per treatment were utilised in each test. After the test solutions had dried, adult whitefly (numbering 20-30) were transferred to the pots, before it was sealed with a lid and turned upside down (whitefly on underside of leaf surface) for 72 hours after treatment at 24 degrees C. with a 16:8 h light/dark regime. Whitefly mortality is evaluated and LC50 values were calculated by LOGIT analysis (using ACSAPwin program).

### b.3) Results

The following compounds, according to the present invention, gave at least 80% control of the Q-biotype (Neonicotinoid resistant) strain of Bemisia tabaci at 200 ppm and exhibited a resistance factor of ≦20: E.001, E.002, E.006, E.008, E.009, E.012, E.013, E.016, E.019, E.020, E.025, E.026, E.028, E.035, E.039, E.040, E.041, E.042, E.044, E.047, E.048, E.051, E.053, E.054, E.058, E.059, E.061, E.063, E.064, E.066, E.067, E.071, E.072.

Thiacloprid and Imidacloprid failed to give 80% control of the Q-biotype (Neonicotinoid resistant) strain of Bemisia tabaci at 200 ppm and both exhibited a resistance factor of >64.

1. A compound of formula I 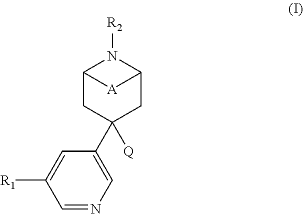
wherein
Q is —C(═S)NR³R⁴ or —C(═NR⁵)SR⁶; where R³ and R⁴ can independently of each other be selected from hydrogen, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), and where R⁵ and R⁶ are independently selected from hydrogen, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl);
A is —CH₂—CH₂— or —CH═CH—;
R¹ is halogen, cyano, C₁-C₃ alkoxy, C₃-C₅ cycloalkyl, —C≡CR⁷; where R⁷ is hydrogen, C₁-C₄alkyl, C₃-C₅cycloalkyl (optionally substituted by one to two substituents independently selected from halogen, methyl and C₁-C₂haloalkyl), tri(C₁-C₂)alkylsilyl; and
R² is hydrogen, formyl, cyano, hydroxy, NH₂, C₁-C₆alkyl (optionally substituted by aryl, aryloxy, heteroaryl or heterocyclyl, which themselves can be optionally substituted by one to three substituents independently selected from halogen, cyano, nitro, C₁-C₄alkyl, C₁-C₄haloalkyl, and C₁-C₄alkoxy), C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₄alkoxyimino(C₁-C₄)alkyl, C₁-C₄haloalkoxy(C₁-C₄)alkyl, C₁-C₆alkoxycarbonyl(C₁-C₆)alkyl, C₁-C₄alkoxy(C₁-C₄)alkoxycarbonyl(C₁-C₆)alkyl, hydroxycarbonyl(C₁-C₆)alkyl, aryloxycarbonyl(C₁-C₆)alkyl (wherein the aryl group can be optionally substituted by one or two substituents independently selected from halogen, cyano, nitro, C₁-C₄alkyl, C₁-C₄ haloalkyl, and C₁-C₄alkoxy), C₁-C₄alkylaminocarbonyl(C₁-C₆)alkyl, di(C₁-C₄alkyl)aminocarbonyl(C₁-C₆)alkyl, C₁-C₄haloalkylaminocarbonyl(C₁-C₆)alkyl, di(C₁-C₄haloalkyl)aminocarbonyl-C₁-C₆alkyl, C₁-C₂alkoxy(C₂-C₄)alkylaminocarbonyl(C₁-C₄)alkyl, C₂-C₆alkenyloxycarbonyl(C₁-C₆)alkyl, C₃-C₆alkynyloxycarbonyl(C₁-C₆)alkyl, (R⁹O)₂(O═)P(C₁-C₆)alkyl where R⁸ is hydrogen, C₁-C₄alkyl or benzyl, C₃-C₇cycloalkyl (optionally substituted by one to three substituents independently selected from C₁-C₄alkyl, C₁-C₄haloalkyl, and C₁-C₄alkoxy and, additionally, one of the ring member units can optionally represent C═O or C═NR⁹ where R⁹ is hydrogen, C₁-C₄alkyl, C₁-C₄haloalkyl, C₁-C₄cyanoalkyl, C₁-C₄alkoxy, or C₃-C₆cycloalkyl), C₃-C₇halocycloalkyl, C₃-C₇cycloalkenyl (optionally substituted by one or two substituents independently selected from C₁-C₄alkyl, and C₁-C₄haloalkyl, and, additionally, one of the ring member units can optionally represent C═O), C₃-C₇halocycloalkenyl, C₁-C₆alkyl-S(═O)n¹(C₁-C₆)alkyl where n¹ is 0, 1 or 2, C₃-C₆alkenyl, C₃-C₆haloalkenyl, aryl(C₃-C₆)alkenyl, C₃-C₆alkynyl, C₃-C₆haloalkynyl, aryl(C₃-C₆)alkynyl, C₃-C₆hydroxyalkynyl, C₁-C₆alkoxycarbonyl (optionally substituted by one to three substituents independently selected from halogen, hydroxy, cyano, C₁-C₄alkoxy, C₁-C₄haloalkyl, and aryl), aryloxycarbonyl (optionally substituted by one to three substituents independently selected from halogen, cyano, nitro, C₁-C₄alkyl, C₁-C₄haloalkyl, C₁-C₄alkoxy), C₃-C₆alkenyloxycarbonyl, C₃-C₆alkynyloxycarbonyl, C₁-C₆alkylcarbonyl, C₁-C₆haloalkylcarbonyl, aminocarbonyl, C₁-C₆alkylaminocarbonyl, di(C₁-C₆)alkylaminocarbonyl, aminothiocarbonyl, C₁-C₆alkylaminothiocarbonyl, di(C₁-C₆)alkylaminothiocarbonyl, C₁-C₆alkoxy, C₃-C₆alkenyloxy, C₃-C₈alkynyloxy, aryloxy (optionally substituted by one to three substituents independently selected from halogen, cyano, nitro, C₁-C₄alkyl, C₁-C₄haloalkyl, and C₁-C₄alkoxy), C₁-C₆alkylamino, di(C₁-C₆)alkylamino, C₃-C₆cycloalkylamino, C₁-C₄alkylthio, C₁-C₄alkylsulfinyl, C₁-C₄alkylsulfonyl, C₁-C₄haloalkylsulfonyl, aryl-S(═O)n² (optionally substituted by one or two substituents independently selected from halogen, nitro, and C₁-C₄alkyl) where n² is 0, 1 or 2, aryl (optionally substituted by one to three substituents independently selected from halogen, cyano, nitro, C₁-C₄alkyl, C₁-C₄haloalkyl, C₁-C₄alkoxy, and C₁-C₄haloalkoxy), heteroaryl (optionally substituted by one to three substituents independently selected from halogen, cyano, nitro, C₁-C₄alkyl, C₁-C₄haloalkyl, C₁-C₄alkoxy, and C₁-C₄haloalkoxy), heterocyclyl (optionally substituted by one to three substituents independently selected from halogen, cyano, nitro, C₁-C₄alkyl, C₁-C₄haloalkyl, C₁-C₄alkoxy, and C₁-C₄haloalkoxy, and, additionally, a ring member unit can optionally represent C═O or C═NR¹⁰ where R¹⁰ is hydrogen, C₁-C₄ alkyl, C₁-C₄ haloalkyl, C₁-C₄ cyanoalkyl, C₁-C₄ alkoxy, or C₃-C₆ cycloalkyl), (C₁-C₆alkylthio)carbonyl, (C₁-C₆alkylthio)thiocarbonyl, C₁-C₆alkyl-S(═O)n³(═NR¹¹)—C₁-C₄alkyl wherein R¹¹ is hydrogen, cyano, nitro, C₁-C₄alkyl and n³ is 0 or 1; or an agrochemically acceptable salt, N-oxide or isomer thereof.

2. A compound according to claim 1 wherein Q is —C(═S)NR³R⁴ or —C(═NR⁵)SR⁶; where R³ and R⁴ are each independently selected from hydrogen, C₁-C₆alkyl (optionally substituted by phenyl which can be optionally substituted by one to three substituents independently selected from halogen, cyano, nitro, C₁-C₄alkyl, C₁-C₄haloalkyl, and C₁-C₄alkoxy), and C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₆alkenyl); and R⁵ and R⁶ are each independently selected from hydrogen, C₁-C₆alkyl (optionally substituted by phenyl which can be optionally substituted by one to three substituents independently selected from halogen, cyano, nitro, C₁-C₄alkyl, C₁-C₄haloalkyl, and C₁-C₄alkoxy), and C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl).

3. A compound according to claim 1 wherein R¹ is halogen, cyano, C₁-C₃alkoxy, C₃-C₅cycloalkyl, or —C≡CR⁷ where R⁷ is hydrogen, C₁-C₄alkyl, C₃-C₅cycloalkyl (which is optionally substituted by one to two substituents independently selected from halogen, methyl and C₁-C₂haloalkyl), or tri(C₁-C₂)alkylsilyl.

4. A compound according to claim 1 wherein R² is hydrogen, C₁-C₆alkyl [optionally substituted by phenyl, phenoxy, heteroaryl (wherein the heteroaryl is pyrimidinyl, pyrazolyl, imidazolyl, thiophenyl, oxazolyl, isoxazolyl, oxadiazolyl, thiazolyl, or thiadiazolyl) or heterocyclyl (wherein heterocyclyl is oxetanyl, thietanyl, tetrahydrofuranyl, [1,3]dioxolanyl, 1-oxo-thietanyl or 1,1-dioxo-thietanyl), which themselves can be optionally substituted by one to two substituents independently selected from halogen, cyano, nitro, C₁-C₄alkyl, C₁-C₄haloalkyl, and C₁-C₄alkoxyl, C₁-C₆haloalkyl (optionally substituted by one to two substituents independently selected from hydroxy, C₁-C₄-alkoxy, tri(C₁-C₄alkyl)silyloxy, C₁-C₂alkylcarbonyloxy, and C₃-C₅alkenyl), C₁-C₆cyanoalkyl, C₁-C₆alkoxy(C₁-C₆)alkyl, C₁-C₄alkoxy(C₁-C₄)alkoxy(C₁-C₄)alkyl, C₁-C₆alkylcarbonyl(C₁-C₆)alkyl, C₁-C₆alkoxycarbonyl(C₁-C₆)alkyl, hydroxycarbonyl(C₁-C₆)alkyl, C₁-C₄alkylaminocarbonyl(C₁-C₆)alkyl, C₁-C₄haloalkylaminocarbonyl(C₁-C₆)alkyl, C₂-C₆alkenyloxycarbonyl(C₁-C₆)alkyl, C₃-C₆cycloalkyl (optionally substituted by one to two substituents independently selected from C₁-C₂alkyl, C₁-C₂haloalkyl, and C₁-C₂alkoxy and, additionally, wherein one of the ring member units can optionally represent C═O), C₃-C₆halocycloalkyl, C₃-C₆cycloalkenyl (wherein one of the ring member units can optionally represent C═O), C₁-C₆alkyl-S(═O)n¹(C₁-C₆)alkyl where n¹ is 0, 1 or 2, C₃-C₆alkenyl, C₃-C₆haloalkenyl, C₃-C₆alkynyl, C₃-C₆haloalkynyl, C₁-C₆alkoxycarbonyl (optionally substituted by halogen, hydroxy, cyano, C₁-C₄alkoxy, C₁-C₄haloalkyl, or phenyl), C₃-C₆alkenyloxycarbonyl, C₁-C₆alkylcarbonyl, C₁-C₆haloalkylcarbonyl, C₁-C₄alkylsulfonyl, C₁-C₄haloalkylsulfonyl, phenyl-S(═O)n² (optionally substituted by one or two substituents independently selected from halogen, nitro, and C₁-C₄alkyl) where n² is 2, heterocyclyl (wherein the heterocyclyl is oxetanyl, thietanyl, tetrahydrofuranyl, tetrahydropyranyl, [1,3]dioxolanyl, 1-oxo-thietanyl or 1,1-dioxo-thietanyl, and wherein the heterocyclyl can be optionally substituted by one to three substituents independently selected from halogen, cyano, C₁-C₂alkyl, C₁-C₂haloalkyl, C₁-C₂alkoxy, and C₁-C₂haloalkoxy, and, additionally, wherein a ring member unit can optionally represent C═O), or C₁-C₄alkyl-S(═O)n³(═NR¹⁷)—C₁-C₄alkyl wherein R¹⁷ is hydrogen, cyano, nitro, C₁-C₄alkyl and n³ is 0 or 1.

5. A compound according to claim 1 wherein Q is —C(═S)NR³R⁴ or —C(═NR⁵)SR⁶; where R³ and R⁴ are each independently hydrogen or C₁-C₆alkyl; R⁵ is hydrogen; and R⁶ is C₁-C₆alkyl.

6. A compound according to claim 1 wherein R¹ is chloro, bromo, cyano, or —C≡CR⁷ where R⁷ is hydrogen.

7. A compound according to claim 1 wherein R² is hydrogen, C₁-C₄alkyl, C₁-C₆haloalkyl, C₁-C₄cyanoalkyl, C₁-C₄alkoxy(C₁-C₄)alkyl, C₁-C₂alkylcarbonyl(C₁-C₂)alkyl, C₁-C₃alkoxycarbonyl(C₁-C₃)alkyl, hydroxycarbonyl(C₁-C₃)alkyl, C₁-C₃alkylaminocarbonyl(C₁-C₃)alkyl, C₁-C₃haloalkylaminocarbonyl(C₁-C₃)alkyl, C₂-C₄alkenyloxycarbonyl(C₁-C₃)alkyl, C₃-C₆cycloalkyl, C₁-C₄alkyl-S(═O)n¹(C₁-C₄)alkyl where n¹ is 0, 1 or 2, C₃-C₆alkenyl, C₃-C₆haloalkenyl, C₃-C₆alkynyl, or heterocyclyl (wherein the heterocyclyl is oxetanyl, thietanyl, tetrahydropyranyl, 1-oxo-thietanyl or 1,1-dioxo-thietanyl).

8. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I) as defined in claim 1.

9. A method according to claim 8 wherein the pests are insects are from the order hemiptera, which insects are resistant to a neonicotinoid insecticide.

10. A method according to claim 8 wherein undesired pests are controlled but beneficial arthropods are not affected.

11. A method according to claim 8 wherein the method comprises applying a compound of formula (I) and one or more beneficial arthropods.

12. A method according to claim 10 wherein the beneficial arthropods are one or more beneficial insects or predatory mites selected from Orius insidiosus, Orius laevigatus, Orius majusculus, Coccinella septempunctata, Adalia bipunctata, Amblydromalus limonicus, Amblyseius andersoni, Amblyseius barkeri, Amblyseius califomicus, Amblyseius cucumeris, Amblyseius montdorensis, Amblyseius swirskii, Phytoseiulus persimilis, Syrphus spp., and Phytoseiulus persimilis.

13. A method according to claim 8 wherein the insects are from the Aleyrodidae family or the Aphididae family.

14. An insecticidal acaricidal, nematicidal or molluscicidal composition comprising an insecticidally acaricidally, nematicidally or molluscicidally effective amount of a compound of formula (I) as defined in claim 1.
